SEQUENCE LISTING (43 sequences)

SEQ ID NO: 1 - protein, 432 aa, Homo sapiens
QVQLVESGGGWQPGRSLRLDCKASGITFSNSGMHWVRQAPGKGLEWVAVRWYDGSKRYYADSVKGRFTISRDNSKNTLFLQMNSLRAEDTAVYYCATNDDYWGQGTLVTVSSASTKGPSVFPLAPCSRSTSESTAALGCLVDYFPEPVTVSWNSGALTSGVHTFPAVLQSSGLYSLSSVVTVPSSSLGTTYTCNVDHKPSNTKVDRVESYGPPCPPCPAPEFLGGPSVFLFPPKPKDTLMISRTPEVTCWVDVSQEDPEVQFNWYYDGVEVHNATKPREEQFNSTYRVVSVLTVLHQDWLNGKEYKCKVSNKGLPSSIEKTISKAGQPREPQVYTLPPSQEEMTKNQVSLTCLVKGFYPSDIAVEWESNGQPEKNYKTTPPVLDSDGSFFLYSRLTVDKSRWQEGNVFSCSVMHEALHNHYTQKSLSLSLGK

SEQ ID NO: 2 - protein, 208 aa, Homo sapiens
EIVLTQSPATLSLSPGERATLSCRASQSVSSYLAWYQQPGQAPRLLIYDASNRATGIPARFSGSGSGTDFTLTISSLEPEDFAVYYCQQSSNWPRTFGQGTKVEIRTVAAPSVFIFPPSDEQLSGTASVVCLLNNFYPREAVQWKVDNALQSGNSQESVTEQDSDSTYSLSSTLTLSKADYEKHKVYACEVTHQGLSSPVTSFNRGEC

SEQ ID NO: 3 - protein, 447 aa, Artificial Sequence
  Pembrolizumab Heavy Chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 218 aa, Artificial Sequence
  Pembrolizumab Light Chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 113 aa, Artificial Sequence
  Humanized Light Chain Variable Domain Sequence
DIVMTQSPDSLAVSLGERATINCKSSQSLWDSGNQKNFLTWYQQKPGQPPKLLIYWTSTRESGVPDRFSGSGSGTDFTLTISSLQAEDVAVYYCQNDYFYPLTFGGGTKVEIK

SEQ ID NO: 6 - protein, 117 aa, Artificial Sequence
  Humanized Heavy Chain Variable Domain Sequence
QVQLVQSGAEVKKPGASVKVSCKASGYTFTSYWINWVRQAPGQGLEQMGNIYPGSSLTNYNEKFKNRVTMTRDTSTSTVYMELSSLRSEDTAVYYCARLLTGTFAYWGQGTLVTVSS

SEQ ID NO: 7 - protein, 113 aa, Artificial Sequence
  Humanized Light Chain Variable Domain Sequence
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPLTFGGGTKVEIK

SEQ ID NO: 8 - protein, 117 aa, Artificial Sequence
  Humanized Heavy Chain Variable Domain
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLLTGTFAYWGQGTLVTVSS

SEQ ID NO: 9 - protein, 113 aa, Artificial Sequence
  Humanized Light Chain Variable Domain
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIK

SEQ ID NO: 10 - protein, 117 aa, Artificial Sequence; identical to SEQ ID NO: 8
  Humanized Heavy Chain Variable Domain

SEQ ID NO: 11 - protein, 113 aa, Artificial Sequence
  Human Light Chain Variable Domain
DIVMTQSPDSLAVSLGERATINCKSSQSLWDSTNQKNFLTWYQQKPGQPPKLLIYWTSTRESGVPDRFSGSGSGTDFTLTISSLQAEDVAVYYCQNDYFYPLTFGGGTKVEIK

SEQ ID NO: 12 - protein, 117 aa, Artificial Sequence; identical to SEQ ID NO: 8
  Human Heavy Chain Variable Domain

SEQ ID NO: 13 - protein, 112 aa, Artificial Sequence
  Humanized Light Chain Variable Domain
DIVTQSPDSLAVSLGERATINCKSSQSLWDSGNQKNFLTWYQQKPGQPPKLLIYWTSTRESGVPDRFSGSGSGTDFTLTISSLQAEDVAVYYCQNDYFYPLTFGGGTKVEIK

SEQ ID NO: 14 - protein, 117 aa, Artificial Sequence
  Humanized Heavy Chain Variable Domain
QVQLVWSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSS

SEQ ID NO: 15 - protein, 113 aa, Artificial Sequence; identical to SEQ ID NO: 7
  Humanized Light Chain Variable Domain

SEQ ID NO: 16 - protein, 117 aa, Artificial Sequence
  Humanized Heavy Chain Variable Domain
QVQLVQSGAEVKKPGASVKVSCKASGYTFTSYWINWVRQAPGQGLEQMGNIYPGSSLTNYNEKFKNRVTMTRDTSTSTVYMELSSLRSEDTAVYYCARLSTGTFAYWGQGTLCTVSS

SEQ ID NO: 17 - protein, 444 aa, Artificial Sequence
  Humanized Heavy Chain
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 443 aa, Artificial Sequence
  Humanized Heavy Chain
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 19 - protein, 221 aa, Artificial Sequence
  Humanized Light Chain
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIKRGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 117 aa, Artificial Sequence
  Humanized Light Chain Variable Domain
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSS

SEQ ID NO: 21 - protein, 117 aa, Artificial Sequence
  Humanized Heavy Chain Variable Domain
QVQLVQSGAEVKKPGASVKVSCKASGYTFTSYWINWVRQAPGQGLEWMGNIWPGSSLTNYNEKFKNRVTMTRDTSTSTVYMELSSLRSEDTAVYYCARLLTGTFAYWGQGTLVTVSS

SEQ ID NO: 22 - protein, 112 aa, Artificial Sequence
  Humanized Light Chain Variable Domain
DIVTQSPDSLAVSLGERATINCKSSQSLWDSTNQKNFLTWYQQKPGQPPKLLIYWTSTRESGVPDRFSGSGSGTDFTLTISSLQAEDVAVYYCQNDYFYPLTFGGGTKVEIK

SEQ ID NO: 23 - protein, 116 aa, Artificial Sequence
  Humanized Heavy Chain Variable Domain
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQTLVTVSS

SEQ ID NO: 24 - protein, 113 aa, Artificial Sequence; identical to SEQ ID NO: 5
  Light Chain Variable Domain

SEQ ID NO: 25 - protein, 117 aa, Artificial Sequence
  Humanized Heavy Chain Variable Domain
QVQLVQSGAEVKKPGASVKVSCKASGYTFTSYWINWVRQAPGQGLEWMGNIYPGSSITNYNEKFKNRVTMTRDTSTSTVYMELSSLRSEDTAVYYCARLTTGTFAYWGQGTLVTVSS

SEQ ID NO: 26 - protein, 113 aa, Artificial Sequence; identical to SEQ ID NO: 7
  Humanized Light Chain Variable Domain

SEQ ID NO: 27 - protein, 117 aa, Artificial Sequence; identical to SEQ ID NO: 25
  Humanized Heavy Chain Variable Domain

SEQ ID NO: 28 - protein, 113 aa, Artificial Sequence; identical to SEQ ID NO: 9
  Humanized Light Chain Variable Domain

SEQ ID NO: 29 - protein, 117 aa, Artificial Sequence; identical to SEQ ID NO: 25
  Humanized Heavy Chain Variable Domain

SEQ ID NO: 30 - protein, 113 aa, Artificial Sequence; identical to SEQ ID NO: 11
  Humanized Light Chain Variable Domain

SEQ ID NO: 31 - protein, 117 aa, Artificial Sequence; identical to SEQ ID NO: 25
  Humanized Heavy Chain Variable Domain

SEQ ID NO: 32 - protein, 113 aa, Artificial Sequence; identical to SEQ ID NO: 5
  Humanized Light Chain Variable Domain

SEQ ID NO: 33 - protein, 117 aa, Artificial Sequence; identical to SEQ ID NO: 21
  Humanized Heavy Chain Variable Domain

SEQ ID NO: 34 - protein, 113 aa, Artificial Sequence; identical to SEQ ID NO: 7
  Humanized Light Chain Variable Domain

SEQ ID NO: 35 - protein, 117 aa, Artificial Sequence; identical to SEQ ID NO: 21
  Humanized Heavy Chain Variable Domain

SEQ ID NO: 36 - protein, 113 aa, Artificial Sequence; identical to SEQ ID NO: 9
  Humanized Light Chain Variable Domain

SEQ ID NO: 37 - protein, 113 aa, Artificial Sequence; identical to SEQ ID NO: 11
  Humanized Light Chain Variable Domain

SEQ ID NO: 38 - protein, 117 aa, Artificial Sequence; identical to SEQ ID NO: 21
  Humanized Heavy Chain Variable Domain

SEQ ID NO: 39 - protein, 432 aa, Artificial Sequence
  Anti-PD-1 fusion protein AMP-224
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWEPKSCDKTHTCPPCPAPELLGGPSVFLFPPKPKDTLMISRTPEVTCWVDVSHEDPEVKFNWYVDGVEVHNAKTKPREEQYNSTYRWSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - DNA, 25 bp, Artificial Sequence
  PD-1 Binding Antagonist Aptamer
gctactgtacatcacgcctctcccc

SEQ ID NO: 41 - DNA, 24 bp, Artificial Sequence
  PD-1 Binding Antagonist Aptamer
ctactgtacatcacgcctctcccc

SEQ ID NO: 42 - DNA, 25 bp, Artificial Sequence
  PD-1 Binding Antagonist Aptamer
gtacagttcccgtccctgcactaca

SEQ ID NO: 43 - DNA, 24 bp, Artificial Sequence
  PD-1 Binding Antagonist Aptamer
gtacagttcccgtcctgcactaca

(19) United States
(12) Patent Application Publication
(10) Pub. No.: US 2020/0368205 A1
(43) Pub. Date: Nov. 26, 2020

(54) METHODS AND COMBINATION THERAPY TO TREAT CANCER

(71) Applicant: ARRAY BIOPHARMA INC., Boulder, CO (US)

(72) Inventors: Patrice A. LEE, Boulder, CO (US); David CHANTRY, Boulder, CO (US); Shannon L. WINSKI, Boulder, CO (US)

(21) Appl. No.: 16/961,133

(22) PCT Filed: Jan. 10, 2018
(86) PCT No.: PCT/US2018/013211

§ 371 Date: Jul. 9, 2020

(51) Int. Cl. A61K 31/4184; A61K 39/395; A61K 9/00; A61P 35/00

(52) U.S. Cl. CPC A61K 31/4184; A61P 35/00; A61K 9/0019; A61K 39/3955

(57) ABSTRACT

This invention relates to a method of treating cancer by administering to a patient in need thereof, over a period of time, therapeutic agents that consist essentially of a MEK inhibitor which is binimetinib or a pharmaceutically acceptable salt thereof, and a PD-1 binding antagonist, to a patient in need thereof.

### FIELD

The present invention relates to methods and combination therapies useful for the treatment of cancer. In particular, this invention relates to methods and combination therapies for treating cancer by administering a combination therapy consisting essentially of a MEK inhibitor which is binimetinib or a pharmaceutically acceptable salt thereof, and a PD-1 binding antagonist. Pharmaceutical uses of the combination of the present invention are also described.

### BACKGROUND

PD-L1 is overexpressed in many cancers and is often associated with poor prognosis (Okazaki T et al., Intern. Immun. 2007 19(7):813) (Thompson R H et al., Cancer Res 2006, 66(7):3381). Interestingly, the majority of tumor infiltrating T lymphocytes predominantly express PD-1, in contrast to T lymphocytes in normal tissues and peripheral blood. PD-1 on tumor-reactive T cells can contribute to impaired antitumor immune responses (Ahmadzadeh et al., Blood 2009 1 14(8): 1537). This may be due to exploitation of PD-L1 signaling mediated by PD-L1 expressing tumor cells interacting with PD-1 expressing T cells to result in attenuation of T cell activation and evasion of immune surveillance (Sharpe et al., Nat Rev 2002, Keir M E et al., 2008 Annu. Rev. Immunol. 26:677). Therefore, inhibition of the PD-L1/PD-1 interaction may enhance CD8+ T cell-mediated killing of tumors.

The inhibition of PD-1 axis signaling through its direct ligands (e.g., PD-L1, PD-L2) has been proposed as a means to enhance T cell immunity for the treatment of cancer (e.g., tumor immunity). Moreover, similar enhancements to T cell immunity have been observed by inhibiting the binding of PD-L1 to the binding partner B7-1. Other advantageous therapeutic treatment regimens could combine blockade of PD-1 receptor/ligand interaction with other anti-cancer agents. There remains a need for such an advantageous therapy for treating, stabilizing, preventing, and/or delaying development of various cancers.

Several PD-1 antagonists, including the PD-1 antibodies nivolumab (Opdivo) and pembrolizumab (Keytruda) were approved by the U.S. Food and Drug Administration (FDA) for the treatment of cancer in recent years.

Mitogen-activated protein kinase (also known as MAP2K, MEK or MAPKK) is a kinase enzyme which phosphorylates mitogen-activated protein kinase (MAPK). The MAPK signaling pathways play critical roles in cell proliferation, survival, differentiation, motility, and angiogenesis. Four distinct MAPK signaling cascades have been identified, one of which involves extracellular signal-regulated kinases ERK1 and ERK2, and their upstream molecules, MEK1 and MEK2. (Akinleye, et al., Journal of Hematology & Oncology 2013 6:27). Inhibitors of MEK1 and MEK2 have been the focus of antitumor drug discoveries.

MEK is a key downstream effector of signaling for multiple receptor tyrosine kinases (RTKs) including VEGF receptors, CSF1R, and the TAM kinases Mer, AXL, and Tyro3. Inhibition of MAPK signaling downstream of these receptors can increase the number of CD8 T⁺ cells present in a tumor through enhanced trafficking (VEGF receptors), blunt the immunosuppressive activity of M2 macrophages (CSF1R), and increase the ability of the immune system to recognize and respond to cancer-associated antigens within the tumor microenvironment (TAM kinases). The latter, which is referred to as immunogenic cell death, would act to promote the expansion of both CD8⁺ effector T cells within the tumor microenvironment and of CD8⁺ central memory T cells within locally draining lymph nodes, further augmenting the anti-tumor immune response (Ann. Rev. Immunol. (2013) 19:598).

There remains a need of finding advantageous combination therapies for treating cancer patients, or particular populations of cancer patients, and potentially with particularized dosing regimens, to improve clinical anti-tumor activity as compared to single agent treatment or double agent treatment, and to optionally improve the combination safety profile.

### SUMMARY

In one embodiment, provided herein is a combination therapy method that consists essentially of administering to a patient in need thereof, over a period of time, therapeutic agents that consist essentially of or consist of therapeutically effective amounts, independently, of a MEK inhibitor which is binimetinib or a pharmaceutically acceptable salt thereof, and a PD-1 binding antagonist.

In one embodiment, provided herein is a method for treating cancer that consists essentially of administering, over a period of time, therapeutic agents that consist essentially of or consist of an amount of a PD-1 binding antagonist and an amount of a MEK inhibitor which is binimetinib or a pharmaceutically acceptable salt thereof to a patient in need thereof, where the amounts together are effective in treating cancer. In one embodiment, binimetinib is crystallized binimetinib.

In some embodiments of any of the methods described herein, before the period of time, the patient was treated with one or more therapeutic agents that did not consist essentially of a PD-1 binding antagonist and an amount of a MEK inhibitor which is binimetinib or a pharmaceutically acceptable salt thereof (e.g., therapeutic agents that consist of a PD-1 binding antagonist and an amount of a MEK inhibitor which is binimetinib or a pharmaceutically acceptable salt thereof). In some embodiments of any of the methods described herein, before the period of time, the patient has been treated with a platinum-based chemotherapeutic agent, and optionally, the patient has been previously determined to be non-responsive to treatment with the platinum-based chemotherapeutic agent, and optionally, has been previously determined to be non-responsive to treatment with the platinum-based chemotherapeutic agent. In some embodiments of any of the methods described herein, before the period of time, the patient has been treated with a BRAF kinase inhibitor (e.g., encorafenib), and optionally, the prior treatment with the BRAF kinase inhibitor (e.g., encorafenib) was unsuccessful. In some embodiments of any of the methods described herein, before the period of time, the patient was treated with a MEK inhibitor (e.g., binimetinib) as a monotherapy, and, optionally, the prior treatment with the MEK inhibitor as a monotherapy was unsuccessful. In some embodiments of any of the methods described herein, before the period of time, the patient was treated with a PD-1 binding antagonist (e.g., nivolumab or pembrolizumab, or a biosimilar thereof) as a monotherapy, and optionally, the prior treatment with the PD-1 binding antagonist (e.g., nivolumab or pembrolizumab, or a biosimilar thereof) was unsuccessful. In any of said embodiments, unsuccessful treatments that have been administered to the patient before the period of time can include, but are not limited to, treatments wherein the patient has failed a prior therapy or has been refractory to such prior therapy, and/or wherein the cancer has metastasized or recurred.

In some embodiments of any of the methods described herein, before the period of time, the patient was treated with one or more of a chemotherapy, a targeted anticancer agent, radiation therapy, and surgery, and optionally, the prior treatment was unsuccessful. In some embodiments of any of the methods described herein, before the period of time, the patient was treated with one or both of a platinum-based chemotherapy and a fluoropyrimidine-containing therapy or therapeutic agent. In some embodiments of any of the methods described herein, before the period of time, the patient was treated with one or both of a EGFR inhibitor and an ALK inhibitor, and optionally, the prior treatment was unsuccessful. In some embodiments of any of the methods described herein, before the period of time, the patient was treated with one or more of folinic acid, fluorouracil, oxaliplatin, and irinotecan, and optionally, the prior treatment was unsuccessful. In some embodiments of any of the methods described herein, before the period of time, the patient was treated with one or more therapeutic agents selected from the group of paclitaxel, gemcitabine, carboplatin, cisplatin, and doxorubicin, and optionally, the prior treatment was unsuccessful.

In some embodiments of any of the methods described herein, after the period of time, the patient is treated with therapeutic agents that do not consist essentially of a PD-1 binding antagonist and an amount of a MEK inhibitor which is binimetinib or a pharmaceutically acceptable salt thereof (e.g., therapeutic agents that consist of a PD-1 binding antagonist and an amount of a MEK inhibitor which is binimetinib or a pharmaceutically acceptable salt thereof)

In some embodiments, administration of the PD-1 binding antagonist and administration of binimetinib or a pharmaceutically acceptable salt thereof during the period of time, occurs at substantially the same time. In some embodiments, administration of the PD-1 binding antagonist to the patient occurs prior to administration of binimetinib or a pharmaceutically acceptable salt thereof to the patient, during the period of time. In some embodiments, administration of binimetinib or a pharmaceutically acceptable salt thereof to the patient occurs prior to administration of the PD-1 binding antagonist to the patient, during the period of time.

In some embodiments, the patient is also administered surgical treatment (e.g., resection of a solid tumor and/or lymph node) and/or a therapy that does not include a BRAF kinase inhibitor (e.g., encorafenib) during the period of time. In some embodiments, the patient is also administered a targeted anticancer agent during the period of time. In some embodiments, the patient is administered radiation therapy during the period of time. In some embodiments, a patient is administered one or more agents to ameliorate side effects of treatment during the period of time (e.g., one or more of corticosteroids, serotonin antagonists, dopamine antagonists, NK-1 inhibitors, cannabinoids, anti-anxiety drugs (e.g., lorazepam or diazepam), antibiotics, anti-fungal agents, colony-stimulating factor, iron supplements, Procrit, epoetin alfa, darbepoetin alfa, anti-emetics, diuretics, NSAIDs, analgesics, methotrexate, anti-diuretics, probiotics, blood pressure medications, anti-nausea agents, laxatives, etc.) during the period of time.

In some embodiments of any of the methods described herein, the patient is not administered a BRAF kinase inhibitor (e.g., encorafenib) during the period of time. In some embodiments of any of the methods described herein, the patient is not administered an additional targeted anticancer agent during the period of time. In some embodiments of any of the methods described herein, the subject is not administered chemotherapy during the period of time. In some embodiments of any of the methods described herein, the subject is not administered a non-MEK kinase targeted inhibitor during the period of time. In some embodiments of any of the methods described herein, the patient is not administered one or more of alkylating agents, anthracyclines, cytoskeletal disruptors (e.g., taxanes), epothilones, histone deacetylase inhibitors, topoisomerase I inhibitors, topoisomerase II inhibitors, nucleotide analogs, nucleotide precursor analogs, peptide antibiotics, platinum-based agents, retinoids, and vinca alkaloids and derivatives thereof, during the period of time. In some embodiments of any of the methods described herein, the patient is not administered a c-MET inhibitor during the period of time. In some embodiments of any of the methods described herein, the subject is not administered a CDK4/6 inhibitor during the period of time. In some embodiments of any of the methods described herein, the patient is not administered a PI3K inhibitor during the period of time. In some embodiments of any of the methods described herein, the subject is not administered a BRAF inhibitor (e.g., encorafenib) during the period of time. In some embodiments of any of the methods described herein, the patient is not administered a FGFR inhibitor during the period of time. In some embodiments of any of the methods described herein, the patient is not administered a BCR-ABL inhibitor during the period of time. In some embodiments of any of the methods described herein, the patient is not administered a different PD-1 binding antagonist than the one administered during the period of time. In some embodiment of any of the methods described herein, the patient is not administered a different MEK inhibitor than the one administered during the period of time. In some embodiments, the patient is not administered a RAS inhibitor during the period of time. In some embodiments, the patient is not administered a CSR-1R inhibitor during the period of time.

In some embodiments, “consisting essentially of,” during the period of time, can include any therapy except for a BRAF kinase inhibitor. In some embodiments, “consisting essentially of,” during the period of time, includes a chemotherapy. In some embodiments, “consisting essentially of,” during the period of time, can include one or more types of chemotherapeutic agents selected from the group of: alkylating agents, anthracyclines, cytoskeletal disruptors (e.g., taxanes), epothilones, histone deacetylase inhibitors, topoisomerase I inhibitors, topoisomerase II inhibitors, nucleotide analogs, nucleotide precursor analogs, peptide antibiotics, platinum-based agents, retinoids, vinca alkaloids, and derivatives. In some embodiments, “consisting essentially of” during the period of time, can include treatment with any targeted chemotherapeutic agent, e.g., a targeted chemotherapeutic agent with the exception of a BRAF kinase inhibitor. In some embodiments, “consisting essentially of,” during the period of time, can include a surgical treatment and/or chemotherapy. In some embodiments, “consisting essentially of” during the period of time, can include treatment with any targeted chemotherapeutic agent, except for one or more of the following: a c-MET inhibitor, a CDK4/6 inhibitor, a PI3K inhibitor, a BRAF inhibitor, a FGFR inhibitor, a MEK inhibitor, and a BCR-ABL inhibitor. In some embodiments, “consisting essentially of” during the period of time can include radiation therapy. In one embodiment, the PD-1 binding antagonist is an anti PD-1 antibody. In one embodiment, the PD-1 binding antagonist is an anti PD-1 antibody selected from nivolumab, pembrolizumab, a biosimilar of nivolumab, and a biosimular of pembrolizumab.

In another embodiment, the invention provides a method for treating cancer comprising or consisting essentially of, administering to a patient in need thereof, during a period of time, therapeutic agents that consist essentially of or consist of an amount of a PD-1 binding antagonist which is nivolumab or a biosimilar thereof, and an amount of a MEK inhibitor which is binimetinib or a pharmaceutically acceptable salt thereof, wherein the amounts together are effective in treating cancer (e.g., during the period of time). In one embodiment, binimetinib is crystallized binimetinib.

In another embodiment, the invention provides a method for treating cancer comprising or consisting essentially of administering to a patient in need thereof, over a period of time, therapeutic agents that consist essentially of or consist of an amount of a PD-1 binding antagonist which is pembrolizumab or a biosimilar thereof, and an amount of a MEK inhibitor which is binimetinib or a pharmaceutically acceptable salt thereof, wherein the amounts together are effective in treating cancer (e.g., during the period of time). In one embodiment, binimetinib is crystallized binimetinib.

In another embodiment, the invention provides a method for treating cancer that comprises or consists essentially of administering, over a period of time, therapeutic agents that consist essentially of or consist of an amount of a PD-1 binding antagonist which is nivolumab or a biosimilar thereof, and an amount of a MEK inhibitor which is binimetinib or a pharmaceutically acceptable salt thereof, to a patient in need thereof, wherein the amounts together are effective in treating cancer (e.g., during the period of time). In one embodiment, binimetinib is crystallized binimetinib.

In another embodiment, the invention provides a method for treating cancer that comprises or consists essentially of administering, over a period of time, therapeutic agents that consist essentially of or consist of a PD-1 binding antagonist which is pembrolizumab or a biosimilar thereof, and an amount of a MEK inhibitor which is binimetinib or a pharmaceutically acceptable salt thereof, to a patient in need thereof, wherein the amounts together are effective in treating cancer (e.g., during the period of time). In one embodiment, binimetinib is crystallized binimetinib.

### BRIEF DESCRIPTION OF DRAWINGS

FIG. 1 is a representative graph of IFNγ-induced MHC class I expression in A375 cells after 72-hour treatment with various concentrations of IFNγ (0.01 ng/mL-1000 ng/mL).

FIG. 2 is a representative graph of IFNγ-induced MHC class I expression in SKMEL-2 cells after 72-hour treatment with various concentrations of IFNγ (0.01 ng/mL-1000 ng/mL).

FIG. 3A is a representative graph of induced MHC class I expression in A375 cells after treatment with 900 nM binimetinib or 900 nM vemurafenib.

FIG. 3B are FACS plots of cell surface MHC class I expression in A375 cells after 72-hour treatment with 900 nM binimetinib.

FIG. 4A is a representative graph of induced MHC class I expression in A375 cells after treatment with 900 nM binimetinib or 900 nM vemurafenib in the presence of 100 ng/mL IFNγ.

FIG. 4B are FACS plots of cell surface MHC class I expression in A375 cells after 72-hour treatment with 900 nM binimetinib in the presence of 100 ng/mL IFNγ.

FIG. 5A is a representative bar graph of induced MHC class I expression in MELJUSO (NRAS Q61L) cells after treatment with binimetinib (0.7 nM, 2.2 nM, 7.3 nM, 24 nM, 81 nM, 271 nM, 902 nM, 3,003 nM, or 10,000 nM). Steady state Cmax=1 μM (26 nM free fraction); Trough*day 15)=310 nM (8.1 nM free fraction).

FIG. 5B is a representative bar graph of induced MHC class I expression in MELJUSO (NRAS Q61L) cells after treatment with binimetinib (0.7 nM, 2.2 nM, 7.3 nM, 24 nM, 81 nM, 271 nM, 902 nM, 3,003 nM, or 10,000 nM) in the presence of 100 ng/mL IFNγ.

FIG. 6A is a representative bar graph of induced MHC class I expression in IPC298 (NRAS Q61L) cells after treatment with binimetinib (0.7 nM, 2.2 nM, 7.3 nM, 24 nM, 81 nM, 271 nM, 902 nM, 3,003 nM, or 10,000 nM). Steady state Cmax=1 μM (26 nM free fraction); Trough*day 15)=310 nM (8.1 nM free fraction).

FIG. 6B is a representative bar graph of induced MHC class I expression in IPC298 (NRAS Q61L) cells after treatment with binimetinib (0.7 nM, 2.2 nM, 7.3 nM, 24 nM, 81 nM, 271 nM, 902 nM, 3,003 nM, or 10,000 nM) in the presence of 100 ng/mL IFNγ.

FIG. 7A is a representative bar graph of induced MHC class I expression in A375 (BRAF V600E) cells after treatment with binimetinib (0.7 nM, 2.2 nM, 7.3 nM, 24 nM, 81 nM, 271 nM, 902 nM, 3,003 nM, or 10,000 nM). Steady state Cmax=1 μM (26 nM free fraction); Trough*day 15)=310 nM (8.1 nM free fraction).

FIG. 7B is a representative bar graph of induced MHC class I expression in A375 (BRAF V600E) cells after treatment with binimetinib (0.7 nM, 2.2 nM, 7.3 nM, 24 nM, 81 nM, 271 nM, 902 nM, 3,003 nM, or 10,000 nM) in the presence of 100 ng/mL IFNγ.

FIG. 8A is a representative bar graph of induced MHC class I expression in HS936.T (NRAS Q61K, BRAF N581K) cells after treatment with binimetinib (0.7 nM, 2.2 nM, 7.3 nM, 24 nM, 81 nM, 271 nM, 902 nM, 3,003 nM, or 10,000 nM). Steady state Cmax=1 μM (26 nM free fraction); Trough*day 15)=310 nM (8.1 nM free fraction).

FIG. 8B is a representative bar graph of induced MHC class I expression in HS936.T (NRAS Q61K, BRAF N581K) cells after treatment with binimetinib (0.7 nM, 2.2 nM, 7.3 nM, 24 nM, 81 nM, 271 nM, 902 nM, 3,003 nM, or 10,000 nM) in the presence of 100 ng/mL IFNγ.

FIG. 9A is a representative bar graph of induced MHC class I expression in MM485 (NRAS Q61R) cells after treatment with binimetinib (0.7 nM, 2.2 nM, 7.3 nM, 24 nM, 81 nM, 271 nM, 902 nM, 3,003 nM, or 10,000 nM). Steady state Cmax=1 μM (26 nM free fraction); Trough*day 15)=310 nM (8.1 nM free fraction).

FIG. 9B is a representative bar graph of induced MHC class I expression in MM485 (NRAS Q61R) cells after treatment with binimetinib (0.7 nM, 2.2 nM, 7.3 nM, 24 nM, 81 nM, 271 nM, 902 nM, 3,003 nM, or 10,000 nM) in the presence of 100 ng/mL IFNγ.

FIG. 10A is a representative bar graph of induced MHC class I expression in SKMEL-2 (NRAS Q61R) cells after treatment with binimetinib (0.7 nM, 2.2 nM, 7.3 nM, 24 nM, 81 nM, 271 nM, 902 nM, 3,003 nM, or 10,000 nM). Steady state Cmax=1 μM (26 nM free fraction); Trough*day 15)=310 nM (8.1 nM free fraction).

FIG. 10B is a representative bar graph of induced MHC class I expression in SKMEL-2 (NRAS Q61R) cells after treatment with binimetinib (0.7 nM, 2.2 nM, 7.3 nM, 24 nM, 81 nM, 271 nM, 902 nM, 3003 nM, or 10 000 nM) in the presence of 100 ng/mL IFNγ.

FIG. 11A is a representative bar graph of induced MHC class I expression in MM415 (NRAS Q61L) cells after treatment with binimetinib (0.7 nM, 2.2 nM, 7.3 nM, 24 nM, 81 nM, 271 nM, 902 nM, 3,003 nM, or 10,000 nM). Steady state Cmax=1 μM (26 nM free fraction); Trough*day 15)=310 nM (8.1 nM free fraction).

FIG. 11B is a representative bar graph of induced MHC class I expression in MM415 (NRAS Q61L) cells after treatment with binimetinib (0.7 nM, 2.2 nM, 7.3 nM, 24 nM, 81 nM, 271 nM, 902 nM, 3,003 nM, or 10,000 nM) in the presence of 100 ng/mL IFNγ.

FIG. 12A is a representative bar graph of induced MHC class I expression in Malme-3M (BRAF V600E) cells after treatment with binimetinib (0.7 nM, 2.2 nM, 7.3 nM, 24 nM, 81 nM, 271 nM, 902 nM, 3,003 nM, or 10,000 nM). Steady state Cmax=1 μM (26 nM free fraction); Trough*day 15)=310 nM (8.1 nM free fraction).

FIG. 12B is a representative bar graph of induced MHC class I expression in Malme-3M (BRAF V600E) cells after treatment with binimetinib (0.7 nM, 2.2 nM, 7.3 nM, 24 nM, 81 nM, 271 nM, 902 nM, 3,003 nM, or 10,000 nM) in the presence of 100 ng/mL IFNγ.

FIG. 13A is a graph showing colorectal cancer CT26 tumor growth in BALB/c mice following treatment with binimetinib (30 mg/kg), anti-PD-1 (100 μg), concomitant treatment of binimetinib and anti-PD-1, or sequential treatment anti-PD-1 followed by binimetinib (n=10).

FIG. 13B are Kaplan-Meier survival curves for the mice in FIG. 13A.

FIG. 13C is a graph showing tumor growth for individual mice in FIG. 13A following sequential administration of anti-PD-1 antibody followed by MEK162 as tumors became resistant to anti-PD-1.

FIG. 14A are representative FACS plots showing the percentage of live CD4⁺ and CD8⁺ cells within the CD45⁺ CD3⁺ cells of a 4T1 tumor, a B16F10 tumor, a P815 tumor, a CT26 tumor, a EMT6 tumor, a LLC1 tumor, and a RENCA tumor.

FIG. 14B is a graph showing the percentage of CD4⁺ CD3⁺ cells across the tumors of FIG. 14A.

FIG. 14C is a graph showing the percentage of CD8⁺ CD3⁺ cells across the tumors of FIG. 14A.

FIG. 14D is a graph showing the percentage of CD11b^{+/hi} cells across the tumors of FIG. 14A.

FIG. 14E is a graph showing the percentage of dendritic cells (DC; CD11c⁺MHC-II⁺) across the tumors of FIG. 14A.

FIG. 14F is a graph showing the percentage of macrophages (F4/80⁺CD11 b⁺) across the tumors of FIG. 14A.

FIG. 14G is a graph showing the percentage of monocytic myeloid-derived suppressor cells (mMDSC; Ly6C^{hi} Ly6G⁻) across the tumors of FIG. 14A.

FIG. 14H is a graph showing the percentage of granulocytic myeloid-derived suppressor cells (gMDSC; Ly6C^{lo} Ly6G⁺) across the tumors of FIG. 14A.

FIG. 15 are bar graphs showing T cell clonality and the T cell fraction within CT26 tumors after treatment (binimetinib, anti-PD-1, concomitant combination therapy or sequential combination therapy).

FIG. 16A is a graph showing melanoma B16F10 tumor growth in C57BL/6 mice following treatment with binimetinib (30 mg/kg), anti-PD-1 (100 μg), concomitant treatment of binimetinib and anti-PD-1, or sequential treatment of binimetinib and anti-PD-1 (n=10).

FIG. 16B are Kaplan-Meier survival curves for the mice in FIG. 16A.

FIG. 17A is a graph showing Cloudman S91 melanoma tumor growth in DBA/2 mice following treatment with binimetinib (30 mg/kg), anti-PD-1 (100 μg), concomitant treatment of binimetinib and anti-PD-1, or sequential treatment of binimetinib and anti-PD-1 (n=10).

FIG. 17B are Kaplan-Meier survival curves for the mice in FIG. 17A.

FIG. 18A is a graph showing renal carcinoma RENCA tumor growth in retired BALB/c mice (23 weeks old) following treatment with binimetinib (30 mg/kg), anti-PD-1 (200 μg), continuous treatment of binimetinib and anti-PD-1, or intermittent treatment of binimetinib and anti-PD-1 (n=12).

FIG. 18B are Kaplan-Meier survival curves for the mice in FIG. 18A.

### DETAILED DESCRIPTION

Combination therapies that include the use of a MEK inhibitor and a PD-1 binding antagonist were discovered herein to provide for improved suppression of anti-tumor immune response and overall improved anti-tumor immune response in a mammal having a cancer (e.g., a cancer that expresses an increased level of PD-1 and optionally, further has aberrant MAPK pathway signaling, e.g., as compared to a control non-cancerous cell).

The present invention may be understood more readily by reference to the following detailed description of the preferred embodiments of the invention and the Examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

### General Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

“About” when used to modify a numerically defined parameter (e.g., the dose of a MEK inhibitor or a PD-1 binding antagonist, or the length of treatment time with a combination therapy described herein) means that the parameter may vary by as much as 10% below or above the stated numerical value for that parameter. For example, a dose of about 5 mg/kg may vary between 4.5 mg/kg and 5.5 mg/kg. “About” when used at the beginning of a listing of parameters is meant to modify each parameter. For example, about 0.5 mg, 0.75 mg or 1.0 mg means about 0.5 mg, about 0.75 mg or about 1.0 mg. Likewise, about 5% or more, 10% or more, 15% or more, 20% or more, and 25% or more means about 5% or more, about 10% or more, about 15% or more, about 20% or more, and about 25% or more.

The phrases “prior to a period of time” or “before a period of time” refer to (1) the completion of administration of surgery and/or radiation treatment to the subject before the first administration of a therapeutic agent during the period of time, and/or (2) the administration of one or more therapeutic agents to the subject before a first administration of a therapeutic agent in the combination therapy described herein during the period of time, such that the one or more therapeutic agents are present in subtherapeutic and/or undetectable levels in the subject at the time the first administration of a therapeutic agent in the combination therapy is performed during the period of time. In some embodiments, the phrase “prior to a period of time” or “before a period of time” refer to the administration of one or more therapeutic agents to the subject before a first administration of a therapeutic agent in the combination therapy during the period of time, such that the one or more therapeutic agents are present in subtherapeutic levels in the subject at the time the first administration of a therapeutic agent in the combination therapy is performed during the period of time. In some embodiments, the phrase “prior to a period of time” or “before a period of time” refer to the administration of one or more therapeutic agents to the subject before a first administration of a therapeutic agent in the combination therapy during the period of time, such that the one or more therapeutic agents are present in undetectable levels in the subject at the time the first administration of a therapeutic agent in the combination therapy is performed during the period of time. In some embodiments, the phrase “prior to a period of time” or “before a period of time” refer to the administration of one or more therapeutic agents to the subject before a first administration of a therapeutic agent in the combination therapy during the period of time, such that the one or more therapeutic agents are present in subtherapeutic and/or undetectable levels in the subject at the time the first administration of a therapeutic agent in the combination therapy is performed during the period of time.

The phrase “pharmaceutically acceptable” indicates that the substance or composition must be compatible chemically and/or toxicologically, with the other ingredients comprising a formulation, and/or the mammal being treated therewith. Some embodiments relate to the pharmaceutically acceptable salts of the compounds described herein. The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined.

“Administration”, “administering”, “treating”, and “treatment,” as it applies to a patient, individual, animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell.

“Treatment” and “treating”, as used in a clinical setting, is intended for obtaining beneficial or desired clinical results. For purposes of this invention, beneficial or desired clinical results include, but are not limited to, one or more of the following: reducing the proliferation of (or destroying) neoplastic or cancerous cells, inhibiting metastasis of neoplastic cells, shrinking or decreasing the size of a tumor, remission of a disease (e.g., cancer), decreasing symptoms resulting from a disease (e.g., cancer), increasing the quality of life of those suffering from a disease (e.g., cancer) (e.g., assessed using FACT-G or EORTC-QLQC30), decreasing the dose of other medications required to treat a disease (e.g., cancer), delaying the progression of a disease (e.g., cancer), and/or prolonging survival of patients having a disease (e.g., cancer). For example, treatment can be the diminishment of one or several symptoms of a disorder, such as cancer. Within the meaning of the present invention, the term “treat” also denotes to arrest, delay the onset (i.e., the period prior to clinical manifestation of a disease) and/or reduce the risk of developing or worsening a disease. “Treatment” can also mean prolonging survival as compared to expected survival if not receiving treatment, for example, an increase in overall survival (OS) compared to a subject not receiving treatment as described herein, and/or an increase in progression-free survival (PFS) compared to a subject not receiving treatment as described herein. The term “treating” can also mean an improvement in the condition of a subject having a cancer, e.g., one or more of a decrease in the size of one or more tumor(s) in a subject, a decrease or no substantial change in the growth rate of one or more tumor(s) in a subject, a decrease in metastasis in a subject, and an increase in the period of remission for a subject (e.g., as compared to the one or more metric(s) in a subject having a similar cancer receiving no treatment or a different treatment, or as compared to the one or more metric(s) in the same subject prior to treatment). Additional metrics for assessing response to a treatment in a subject having a cancer are disclosed herein below.

The term “subject” includes any organism, preferably an animal, more preferably a mammal (e.g., rat, mouse, dog, cat, and rabbit) and most preferably a human.

A “patient” to be treated according to this invention includes any warm-blooded animal, such as, but not limited to human, monkey or other lower-order primate, horse, dog, rabbit, guinea pig, or mouse. In one embodiment the patient is human. In one embodiment, the patient is a pediatric patient. Those skilled in the medical art are readily able to identify individuals who are afflicted with cancer and who are in need of treatment.

The term “pediatric patient” as used herein refers to a patient under the age of 16 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First LR. Pediatric Medicine, 2nd Ed. Baltimore: Williams & Wilkins; 1994.

The terms “treatment regimen” and “dosing regimen” are used interchangeably to refer to the dose and timing of administration of each therapeutic agent in a combination of the invention.

“Ameliorating” means a lessening or improvement of one or more symptoms as compared to not administering a treatment. “Ameliorating” also includes shortening or reduction in duration of a symptom.

The term “regulatory agency” is a country's agency for the approval of the medical use of pharmaceutical agents with the country. For example, a non-limiting example of a regulatory agency is the U.S. Food and Drug Administration (FDA).

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also antigen binding fragments thereof (such as Fab, Fab′, F (ab′)₂, Fv), single chain (scFv) and domain antibodies (including, for example, shark and camelid antibodies), and fusion proteins comprising an antibody, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen binding fragment” or “antigen binding portion” of an antibody, as used herein, refers to one or more fragments of an intact antibody that retain the ability to specifically bind to a given antigen (e.g., PD-1). Antigen binding functions of an antibody can be performed by fragments of an intact antibody. Examples of binding fragments encompassed within the term “antigen binding fragment” of an antibody include Fab; Fab′; F (ab′) 2; an Fd fragment consisting of the VH and CH1 domains; an Fv fragment consisting of the VL and VH domains of a single arm of an antibody; a single domain antibody (dAb) fragment (Ward et al., Nature 341:544-546, 1989), and an isolated complementarity determining region (CDR).

An antibody, an antibody conjugate, or a polypeptide that “preferentially binds” or “specifically binds” (used interchangeably herein) to a target (e.g., PD-1 protein) is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a PD-1 epitope is an antibody that binds this epitope with greater affinity, avidity, more readily, and/or with greater duration than it binds to other PD-1 epitopes or non-PD-1 epitopes. It is also understood that by reading this definition, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

A “variable region” of an antibody refers to the variable region of the antibody light chain or the variable region of the antibody heavy chain, either alone or in combination. As known in the art, the variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al., 1997, J. Molec. Biol. 273:927-948). As used herein, a CDR may refer to CDRs defined by either approach or by a combination of both approaches.

A “CDR” of a variable domain are amino acid residues within the variable region that are identified in accordance with the definitions of the Kabat, Chothia, the accumulation of both Kabat and Chothia, AbM, contact, and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., Nature 342:877-883, 1989. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., J. Mol. Biol., 262:732-745, 1996. In another approach, referred to herein as the “conformational definition” of CDRs, the positions of the CDRs may be identified as the residues that make enthalpic contributions to antigen binding. See, e.g., Makabe et al., Journal of Biological Chemistry, 283:1156-1166, 2008. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

“Isolated antibody” and “isolated antibody fragment” refers to the purification status and in such context means the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1 below.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

The term “PD-1 binding antagonist” as used herein refers to a molecule that binds specifically to PD-1 and decreases the interaction of PD-1 with one or more of its binding partners, such as PD-L1 and/or PD-L2. For example, PD-1 binding antagonists include anti-PD-1 antibodies, antigen binding fragments thereof, immunoadhesins, aptamers, fusion proteins, and oligopeptides. In one embodiment, a PD-1 binding antagonist reduces the negative co-stimulatory signal mediated by or through cell surface proteins expressed on T lymphocytes mediated signaling through PD-1 so as render a dysfunctional T-cell less dysfunctional. In some embodiments, the PD-1 binding antagonist is an anti-PD-1 antibody. In one embodiment, the PD-1 binding antagonist is an anti-PD-1 antibody selected from nivolumab, pembrolizumab, a biosimilar of nivolumab, and a biosimular of pembrolizumab. In one embodiment, the PD-1 binding antagonist is nivolumab or a biosimilar thereof. In one embodiment, the PD-1 binding antagonist is pembrolizumab or a biosimilar thereof.

An anti-PD-1 antibody as described herein can also be an antigen-binding antibody fragment of nivolumab or a biosimilar thereof, or an antigen-binding antibody fragment of pembrolizumab or a biosimilar thereof.

In some examples, an anti-PD-1 antibody can be a biosimilar of nivolumab, or a biosimilar of pembrolizumab.

A “biosimilar” means an antibody or antigen-binding fragment that has the same primary amino acid sequence as compared to a reference antibody (e.g., nivolumab or pembrolizumab) and optionally, may have detectable differences in post-translation modifications (e.g., glycosylation and/or phosphorylation) as compared to the reference antibody (e.g., a different glycoform).

In some embodiments, a biosimilar is an antibody or antigen-binding fragment thereof that has a light chain that has the same primary amino acid sequence as compared to a reference antibody (e.g., nivolumab or pembrolizumab) and a heavy chain that has the same primary amino acid sequence as compared to the reference antibody. In some examples, a biosimilar is an antibody or antigen-binding fragment thereof that has a light chain that includes the same light chain variable domain sequence as a reference antibody (e.g., nivolumab or pembrolizumab) and a heavy chain that includes the same heavy chain variable domain sequence as a reference antibody. In some embodiments, a biosimilar can have a similar glycosylation pattern as compared to the reference antibody (e.g., nivolumab or pembrolizumab). In other embodiments, a biosimilar can have a different glycosylation pattern as compared to the reference antibody (e.g., nivolumab or pembrolizumab).

Table 2 below provides a list of the amino acid sequences of exemplary PD-1 binding antagonists for use in the treatment method, medicaments and uses of the present invention. CDRs are underlined for mAb7 and mAb15. The mAB7 is also known as RN888 or PF-6801591. mAb7 (aka RN888) and mAb15 are disclosed in International Patent Publication No. WO2016/092419, the disclosure of which is hereby incorporated by reference in its entirety.

TABLE 2

| | |
|---|---|
| Nivolumab, MDX1106, full | QVQLVESGGGWQPGRSLRLDCKASGITFSNSGMHWVRQ |
| length heavy chain From | APGKGLEWVAVrWYDGSKRYYADSVKGRFTISRDNSKNT |
| WO 2006/121168 | LFLQMNSLRAEDTAVYYCATNDDYWGQGTLVTVSSASTK |
| | GPSVFPLAPCSRSTSESTAALGCLVDYFPEPVTVSWNSG |
| | ALTSGVHTFPAVLQSSGLYSLSSVVTVPSSSLGTTYTCNV |
| | DHKPSNTKVDRVESYGPPCPPCPAPEFLGGPSVFLFPPK |
| | PKDTLMISRTPEVTCWVDVSQEDPEVQFNWYYDGVEVHN |
| | ATKPREEQFNSTYRVVSVLTVLHQDWLNGKEYKCKVSNK |
| | GLPSSIEKTISKA |
| | GQPREPQVYTLPPSQEEMTKNQVSLTCLVKGFYPSDIAVE |
| | WESNGQPEKNYKTTPPVLDSDGSFFLYSRLTVDKSRWQE |
| | GNVFSCSVMHEALHNHYTQKSLSLSLGK |
| | (SEQ ID NO: 1) |
| Nivolumab, MDX1106, full | EIVLTQSPATLSLSPGERATLSCRASQSVSSYLAWYQQPG |
| length light chain From | QAPRLLIYDASNRATGIPARFSGSGSGTDFTLTISSLEPED |
| WO 2006/121168 | FAVYYCQQSSNWPRTFGQGTKVEIRTVAAPSVFIFPPSDE |
| | QLSGTASVVCLLNNFYPREAVQWKVDNALQSGNSQESVT |
| | EQDSDSTYSLSSTLTLSKADYEKHKVYACEVTHQGLSSPV |
| | T SFNRGEC (SEQ ID NO: 2) |
| Pembrolizumab, MK3475, | QVQLVQSGVEVKKPGASVKVSCKASGYTFTNYYMYWV |
| full length heavy chain | RQAPGQGLEWMGGINPSNGGTNFNEKFKNRVTLTTDS |
| From WO 2009/114335 | STTTAYMELKSLQFDDTAVYYCARRDYRFDMGFDYWG |
| | QGTTVTVSSASTKGPSVFPLAPCSRSTSESTAALGCLVK |
| | DYFPEPVTVSWNSGALTSGVHTFPAVLQSSGLYSLSSV |
| | VTVPSSSLGTKTYTCNVDHKPSNTKVDKRVESKYGPPC |
| | PPCPAPEFLGGPSVFLFPPKPKDTLMISRTPEVTCVVVD |
| | VSQEDPEVQFNWYVDGVEVHNAKTKPREEQFNSTYRV |
| | VSVLTVLHQDWLNGKEYKCKVSNKGLPSSIEKTISKAKG |
| | QPREPQVYTLPPSQEEMTKNQVSLTCLVKGFYPSDIAV |
| | EWESNGQPENNYKTTPPVLDSDGSFFLYSRLTVDKSR |
| | WQEGNVFSCSVMHEALHNHYTQKSLSLSLGK (SEQ ID |
| | NO: 3) |
| Pembrolizumab, MK3475, | EIVLTQSPATLSLSPGERATLSCRASKGVSTSGYSYLHW |
| full length light chain | YQQKPGQAPRLLIYLASYLESGVPARFSGSGSGTDFTLT |
| From WO 2009/114335 | ISSLEPEDFAVYYCQHSRDLPLTFGGGTKVEIKRTVAAP |
| | SVFIFPPSDEQLKSGTASVVCLLNNFYPREAKVQWKVD |
| | NALQSGNSQESVTEQDSKDSTYSLSSTLTLSKADYEKH |
| | KVYACEVTHQGLSSPVT KSFNRGEC (SEQ ID NO: 4) |
| mAb1 light chain variable | DIVMTQSPDSLAVSLGERATINCKSSQSLWDSGNQKNF |
| domain with CDRs in | LTWYQQKPGQPPKLLIYWTSTRESGVPDRFSGSGSGT |
| bold (from WO 16/92419) | DFTLTISSLQAEDVAVYYCQNDYFYPLTFGGGTKVEIK |
| | (SEQ ID NO: 5) |
| mAb1 heavy chain variable | QVQLVQSGAEVKKPGASVKVSCKASGYTFTSYWINWVRQ |
| domain with CDRs | APGQGLEQMGNIYPGSSLTNYNEKFKNRVTMTRDTSTST |
| underlined (from WO | VYMELSSLRSEDTAVYYCARLLTGTFAYWGQGTLVTVSS |
| 16/92419) | (SEQ ID NO: 6) |
| mAb2 light chain variable | DIVMTQSPDSLAVSLGERATINCKSSQSLWDSGNQKNFLT |
| domain with CDRs | WYQQKPGQPPKLLIYWTSYRESGVPDRFSGSGSGTDFTL |
| underlined (from WO | TISSLQAEDVAVYYCQNDYFYPLTFGGGTKVEIK |
| 16/92419) | (SEQ ID NO: 7) |
| mAb2 heavy chain variable | QVQLVQSGAEVKKPGASVKVSCKASGYTFTSYWINWVRQ |
| domain with CDRs | APGQGLEWMGNIYPGSSLTNYNEKFKNRVTMTRDTSTST |
| underlined (from WO | VYMELSSLRSEDTAVYYCARLLTGTFAYWGQGTLVTVSS |
| 16/92419) | (SEQ ID NO: 8) |
| mAb3 light chain variable | DIVMTQSPDSLAVSLGERATINCKSSQSLWDSGNQKNFLT |
| domain with CDRs | WYQQKPGQPPKLLIYWTSYRESGVPDRFSGSGSGTDFTL |
| underlined (from WO | TISSLQAEDVAVYYCQNDYFYPHTFGGGTKVEIK |
| 16/92419) | (SEQ ID NO: 9) |
| mAb3 heavy chain | QVQLVQSGAEVKKPGASVKVSCKASGYTFTSYWINWVRQ |
| variable domain with | APGQGLEWMGNIYPGSSLTNYNEKFKNRVTMTRDTSTST |
| CDRs underlined (from | VYMELSSLRSEDTAVYYCARLLTGTFAYWGQGTLVTVSS |
| WO 16/92419) | (SEQ ID NO: 10) |
| mAb4 light chain variable | DIVMTQSPDSLAVSLGERATINCKSSQSLWDSTNQKNFLT |
| domain with CDRs | WYQQKPGQPPKLLIYWTSTRESGVPDRFSGSGSGTDFTL |
| underlined (from WO | TISSLQAEDVAVYYCQNDYFYPLTFGGGTKVEIK |
| 16/92419) | (SEQ ID NO: 11) |
| mAb4 heavy chain | QVQLVQSGAEVKKPGASVKVSCKASGYTFTSYWINWVRQ |
| variable domain with | APGQGLEWMGNIYPGSSLTNYNEKFKNRVTMTRDTSTST |
| CDRs underlined (from | VYMELSSLRSEDTAVYYCARLLTGTFAYWGQGTLVTVSS |
| WO 16/92419) | (SEQ ID NO: 12) |
| mAb5 light chain variable | DIVTQSPDSLAVSLGERATINCKSSQSLWDSGNQKNFLT |
| domain with CDRs | WYQQKPGQPPKLLIYWTSTRESGVPDRFSGSGSGTDFTL |
| underlined (from WO | TISSLQAEDVAVYYCQNDYFYPLTFGGGTKVEIK |
| 16/92419) | (SEQ ID NO: 13) |
| mAb5 heavy chain | QVQLVWSGAEVKKPGASVKVSCKASGYTFTSYWINWVR |
| variable domain with | QAPGQGLEWMGNIYPGSSLTNYNEKFKNRVTMTRDTSTS |
| CDRs underlined (from | TVYMELSSLRSEDTAVYYCARLSTGTFAYWGQGTLVTVSS |
| WO 16/92419) | (SEQ ID NO: 14) |
| mAb6 light chain variable | DIVMTQSPDSLAVSLGERATINCKSSQSLWDSGNQKNFLT |
| domain with CDRs | WYQQKPGQPPKLLIYWTSYRESGVPDRFSGSGSGTDFTL |
| underlined (from WO | TISSLQAEDVAVYYCQNDYFYPLTFGGGTKVEIK |
| 16/92419) | (SEQ ID NO: 15) |
| mAb6 heavy chain | QVQLVQSGAEVKKPGASVKVSCKASGYTFTSYWINWVRQ |
| variable domain with | APGQGLEQMGNIYPGSSLTNYNEKFKNRVTMTRDTSTST |
| CDRs underlined (from | VYMELSSLRSEDTAVYYCARLSTGTFAYWGQGTLCTVSS |
| WO 16/92419) | (SEQ ID NO: 16) |
| mAb7(also known as | QVQLVQSGAEVKKPGASVKVSCKASGYTFTSYWINWVRQ |
| RN888) or mAb15 full- | APGQGLEWMGNIYPGSSLTNYNEKFKNRVTMTRDTSTST |
| length heavy chain | VYMELSSLRSEDTAVYYCARLSTGTFAYWGQGTLVTVSS |
| | ASTKGPSVFPLAPCSRSTSESTAALGCLVKDYFPEPVTVS |
| | WNSGALTSGVHTFPAVLQSSGLYSLSSVVTVPSSSLGTKT |
| | YTCNVDHKPSNTKVDKRVESKYGPPCPPCPAPEFLGGPS |
| | VFLFPPKPKDTLMISRTPEVTCVVVDVSQEDPEVQFNWYV |
| | DGVEVHNAKTKPREEQFNSTYRVVSVLTVLHQDWLNGKE |
| | YKCKVSNKGLPSSIEKTISKAKGQPREPQVYTLPPSQEEM |
| | TKNQVSLTCLVKGFYPSDIAVEWESNGQPENNYKTTPPVL |
| | DSDGSFFLYSRLTVDKSRWQEGNVFSCSVMHEALHNHYT |
| | QKSLSLSLGK (SEQ ID NO: 17) |
| mAb7 or mAb 15 full- | QVQLVQSGAEVKKPGASVKVSCKASGYTFTSYWINWVRQ |
| length heavy chain | APGQGLEWMGNIYPGSSLTNYNEKFKNRVTMTRDTSTST |
| without the C-terminal | VYMELSSLRSEDTAVYYCARLSTGTFAYWGQGTLVTVSS |
| lysine | ASTKGPSVFPLAPCSRSTSESTAALGCLVKDYFPEPVTVS |
| | WNSGALTSGVHTFPAVLQSSGLYSLSSVVTVPSSSLGTKT |
| | YTCNVDHKPSNTKVDKRVESKYGPPCPPCPAPEFLGGPS |
| | VFLFPPKPKDTLMISRTPEVTCVVVDVSQEDPEVQFNWYV |
| | DGVEVHNAKTKPREEQFNSTYRVVSVLTVLHQDWLNGKE |
| | YKCKVSNKGLPSSIEKTISKAKGQPREPQVYTLPPSQEEM |
| | TKNQVSLTCLVKGFYPSDIAVEWESNGQPENNYKTTPPVL |
| | DSDGSFFLYSRLTVDKSRWQEGNVFSCSVMHEALHNHYT |
| | QKSLSLSLG (SEQ ID NO: 18) |
| mAb7 full-length light | DIVMTQSPDSLAVSLGERATINCKSSQSLWDSGNQKNFLT |
| chain | WYQQKPGQPPKLLIYWTSYRESGVPDRFSGSGSGTDFTL |
| | TISSLQAEDVAVYYCQNDYFYPHTFGGGTKVEIKRGTVAA |
| | PSVFIFPPSDEQLKSGTASVVCLLNNFYPREAKVQWKVDN |
| | ALQSGNSQESVTEQDSKDSTYSLSSTLTLSKADYEKHKVY |
| | ACEVTHQGLSSPVTKSFNRGEC (SEQ ID NO: 19) |
| mAb7 light chain variable | QVQLVQSGAEVKKPGASVKVSCKASGYTFTSYWINWVRQ |
| region | APGQGLEWMGNIYPGSSLTNYNEKFKNRVTMTRDTSTST |
| | VYMELSSLRSEDTAVYYCARLSTGTFAYWGQGTLVTVSS |
| | (SEQ ID NO: 20) |
| mAB7 and mAB15 heavy | QVQLVQSGAEVKKPGASVKVSCKASGYTFTSYWINWVRQ |
| chain variable region | APGQGLEWMGNIWPGSSLTNYNEKFKNRVTMTRDTSTST |
| | VYMELSSLRSEDTAVYYCARLLTGTFAYWGQGTLVTVSS |
| | (SEQ ID NO: 21) |
| mAb8 light chain variable | DIVTQSPDSLAVSLGERATINCKSSQSLWDSTNQKNFLTW |
| domain with CDRs in | YQQKPGQPPKLLIYWTSTRESGVPDRFSGSGSGTDFTLTI |
| bold (from WO 16/92419) | SSLQAEDVAVYYCQNDYFYPLTFGGGTKVEIK (SEQ ID |
| | NO: 22) |
| mAb8 heavy chain | QVQLVQSGAEVKKPGASVKVSCKASGYTFTSYWINWVRQ |
| variable domain with | APGQGLEWMGNIYPGSSLTNYNEKFKNRVTMTRDTSTST |
| CDRs in bold (from WO | VYMELSSLRSEDTAVYYCARLSTGTFAYWGQTLVTVSS |
| 16/92419) | (SEQ ID NO: 23) |
| mAb9 light chain variable | DIVMTQSPDSLAVSLGERATINCKSSQSLWDSGNQKNF |
| domain with CDRs in | LTWYQQKPGQPPKLLIYWTSTRESGVPDRFSGSGSGT |
| bold (from WO 16/92419) | DFTLTISSLQAEDVAVYYCQNDYFYPLTFGGGTKVEIK |
| | (SEQ ID NO: 24) |
| mAb9 heavy chain | QVQLVQSGAEVKKPGASVKVSCKASGYTFTSYWINWVRQ |
| variable domain with | APGQGLEWMGNIYPGSSITNYNEKFKNRVTMTRDTSTST |
| CDRs in bold (from WO | VYMELSSLRSEDTAVYYCARLTTGTFAYWGQGTLVTVSS |
| 16/92419) | (SEQ ID NO: 25) |
| mAb10 light chain | DIVMTQSPDSLAVSLGERATINCKSSQSLWDSGNQKNFLT |
| variable domain with | WYQQKPGQPPKLLIYWTSYRESGVPDRFSGSGSGTDFTL |
| CDRs in bold (from WO | TISSLQAEDVAVYYCQNDYFYPLTFGGGTKVEIK |
| 16/92419) | (SEQ ID NO: 26) |
| mAb10 heavy chain | QVQLVQSGAEVKKPGASVKVSCKASGYTFTSYWINWVRQ |
| variable domain with | APGQGLEWMGNIYPGSSITNYNEKFKNRVTMTRDTSTST |
| CDRs in bold (from WO | VYMELSSLRSEDTAVYYCARLTTGTFAYWGQGTLVTVSS |
| 16/92419) | (SEQ ID NO: 27) |
| mAb11 light chain | DIVMTQSPDSLAVSLGERATINCKSSQSLWDSGNQKNFLT |
| variable domain with | WYQQKPGQPPKLLIYWTSYRESGVPDRFSGSGSGTDFTL |
| CDRs in bold (from WO | TISSLQAEDVAVYYCQNDYFYPHTFGGGTKVEIK |
| 16/92419) | (SEQ ID NO: 28) |
| mAb11 heavy chain | QVQLVQSGAEVKKPGASVKVSCKASGYTFTSYWINWVRQ |
| variable domain with | APGQGLEWMGNIYPGSSITNYNEKFKNRVTMTRDTSTST |
| CDRs in bold (from WO | VYMELSSLRSEDTAVYYCARLTTGTFAYWGQGTLVTVSS |
| 16/92419) | (SEQ ID NO: 29) |
| mAb12 light chain | DIVMTQSPDSLAVSLGERATINCKSSQSLWDSTNQKNFLT |
| variable domain with | WYQQKPGQPPKLLIYWTSTRESGVPDRFSGSGSGTDFTL |
| CDRs in bold (from WO | TISSLQAEDVAVYYCQNDYFYPLTFGGGTKVEIK |
| 16/92419) | (SEQ ID NO: 30) |
| mAb12 heavy chain | QVQLVQSGAEVKKPGASVKVSCKASGYTFTSYWINWVRQ |
| variable domain with | APGQGLEWMGNIYPGSSITNYNEKFKNRVTMTRDTSTST |
| CDRs in bold (from WO | VYMELSSLRSEDTAVYYCARLTTGTFAYWGQGTLVTVSS |
| 16/92419) | (SEQ ID NO: 31) |
| mAb13 light chain | DIVMTQSPDSLAVSLGERATINCKSSQSLWDSGNQKNFLT |
| variable domain with | WYQQKPGQPPKLLIYWTSTRESGVPDRFSGSGSGTDFTL |
| CDRs in bold (from WO | TISSLQAEDVAVYYCQNDYFYPLTFGGGTKVEIK |
| 16/92419) | (SEQ ID NO: 32) |
| mAb13 heavy chain | QVQLVQSGAEVKKPGASVKVSCKASGYTFTSYWINWVRQ |
| variable domain with | APGQGLEWMGNIWPGSSLTNYNEKFKNRVTMTRDTSTS |
| CDRs in bold (from WO | TVYMELSSLRSEDTAVYYCARLLTGTFAYWGQGTLVTVSS |
| 16/92419) | (SEQ ID NO: 33) |
| mAb14 light chain | DIVMTQSPDSLAVSLGERATINCKSSQSLWDSGNQKNFLT |
| variable domain with | WYQQKPGQPPKLLIYWTSYRESGVPDRFSGSGSGTDFTL |
| CDRs in bold (from WO | TISSLQAEDVAVYYCQNDYFYPLTFGGGTKVEIK |
| 16/92419) | (SEQ ID NO: 34) |
| mAb14 heavy chain | QVQLVQSGAEVKKPGASVKVSCKASGYTFTSYWINWVRQ |
| variable domain with | APGQGLEWMGNIWPGSSLTNYNEKFKNRVTMTRDTSTS |
| CDRs in bold (from WO | TVYMELSSLRSEDTAVYYCARLLTGTFAYWGQGTLVTVSS |
| 16/92419) | (SEQ ID NO: 35) |
| mAb15 light chain | DIVMTQSPDSLAVSLGERATINCKSSQSLWDSGNQKNFLT |
| variable region | WYQQKPGQPPKLLIYWTSYRESGVPDRFSGSGSGTDFTL |
| | TISSLQAEDVAVYYCQNDYFYPHTFGGGTKVEIK |
| | (SEQ ID NO: 36) |
| mAb16 light chain | DIVMTQSPDSLAVSLGERATINCKSSQSLWDSTNQKNFLT |
| variable domain with | WYQQKPGQPPKLLIYWTSTRESGVPDRFSGSGSGTDFTL |
| CDRs in bold (from WO | TISSLQAEDVAVYYCQNDYFYPLTFGGGTKVEIK |
| 16/92419) | (SEQ ID NO: 37) |
| mAb16 heavy chain | QVQLVQSGAEVKKPGASVKVSCKASGYTFTSYWINWVRQ |
| variable domain with | APGQGLEWMGNIWPGSSLTNYNEKFKNRVTMTRDTSTS |
| CDRs in bold (from WO | TVYMELSSLRSEDTAVYYCARLLTGTFAYWGQGTLVTVSS |
| 16/92419) | (SEQ ID NO: 38) |
| AMP224, without signal | LFTVTVPKELYIIEHGSNVTLECNFDTGSHVNLGAITASLQK |
| sequence From WO | VENDTSPHRERATLLEEQLPLGKASFHIPQVQVRDEGQYQ |
| 2010/027827 and | CIIIYGVA |
| WO 2011/066342 | WDYKYLTLKVKASYRKINTHILKVPETDEVELTCQATGYPL |
| | AEVSWPNVSVPANTSHSRTPEGLYQVTSVLRLKPPPGRN |
| | FSCVFWNTHVRELTLASIDLQSQMEPRTHPTWEPKSCDK |
| | THTCPPCPAPELLGGPSVFLFPPKPKDTLMISRTPEVTCW |
| | VDVSHEDPEVKFNWYVDGVEVHNAKTKPREEQYNSTYR |
| | WSVLTVLHQDWLNGKEYKCKVSNKALPAPIEKTISKAKGQ |
| | PREPQVYTLPPSRDELTKNQV SLTCLVKGFY |
| | PSDIAVEWES NGQPENNYKT TPPVLDSDGS |
| | FFLYSKLTVDKSRWQQGNVFSCSVMHEALHNHYTQKSLS |
| | LSPGK (SEQ ID NO: 39) |

Further examples of an anti-PD-1 antibody include CT-011 (pidilizumab, which is described in WO 09/101611), IBI-308, mDX-400, BGB-108, MEDI-0680, SHR-1210, PF-06801591, PDR-001, GB-226, STI-1110, MEDI-0680 (AMP-514), PDR001, REGN2810, BGB-108, and BGB-A317, or a biosimilar of any of these antibodies. Additional exemplary anti-PD-1 antibodies are described in U.S. Patent Application Nos. 2017/0349666, 2017/0327590, 2017/0327582, 2017/0320949, 2017/0290913, 2017/0290808, 2017/0275705, 2017/0274073, 2017/0267762, 2017/0247456, 2017/0239351, 2017/0239351, 2017/0198037, 2017/0166641, 2017/0121409, 2017/0112925, 2017/0112925, 2017/0088615, 2017/0052188, 2017/0044260, 2017/0044256, 2017/0039345, 2017/0037127, 2017/0037125, 2017/0021019, 2017/0008971, 2017/0000885, 2016/0376367, 2016/0362492, 2016/0312297, 2016/0312295, 2016/0304969, 2016/0304606, 2016/0303231, 2016/0289315, 2016/0257752, 2016/0222118, 2016/0222113, 2016/0206719, 2016/0206719, 2016/0193334, 2016/0166685, 2016/0158360, 2016/0130348, 2016/0130345, 2016/0075783, 2016/0068586, 2016/0052990, 2016/0051672, 2016/0039903, 2016/0031990, 2016/0022814, 2016/0002334, 2015/0265705, 2015/0250837, 2015/0232555, 2015/0216970, 2015/0210772, 2015/0210769, 2015/0203579, 2015/0190506, 2015/0152180, 2015/0125955, 2015/0118245, 2015/0118234, 2015/0071910, 2014/0356363, 2014/0348743, 2014/0341902, 2014/0335093, 2014/0294852, 2014/0271684, 2014/0234296, 2014/0227262, 2014/0178370, 2014/0044738, 2013/0230514, 2013/0164294, 2013/0156774, 2013/0133091, 2013/0109843, 2013/0108651, 2013/0095098, 2013/0022629, 2013/0022600, 2012/0114649, 2012/0114648, 2012/0039906, 2011/0280878, 2011/0229461, 2011/0195068, 2011/0171220, 2011/0171215, 2011/0159023, 2011/0123550, 2011/0008777, 2011/0008369, 2010/0285013, 2010/0266617, 2010/0055102, 2009/0263865, 2009/0217401, 2009/0076250, 2009/0028857, 2008/0311117, 2008/0025979, 2007/0092504, 2006/0210567, 2006/0034826, 2004/0241745, and 2004/0213795.

In some embodiments, a PD-1 binding antagonist can be a fusion protein (e.g., an immunoadhesin, e.g., AMP-224, also called B7-DCIg, which is described in WO 10/027827 and WO 11/066342). For example, an immunoadhesin can include an extracellular or PD-1 binding portion of PD-L1 or PD-L2 fused to an antibody constant region (e.g., an Fc region of an immunoglobulin (e.g., a human immunoglobulin) sequence).

In some embodiments, a PD-1 binding antagonist can be an aptamer. Non-limiting examples of PD-1 binding antagonists that are aptamers are described in, e.g., US 2017/0218369. Additional examples of aptamers that are PD-1 binding antagonists are described in Prodeus et al., Mol. Ther. Nucleic Acids 4:e237, 2015; Wang et al., doi: 10.1016/j.biochi.2017.09.006 Biochimie. For example, a PD-1 binding antagonist that is an aptamer can include a sequence of one of:

(SEQ ID NO: 40)
GCTACTGTACATCACGCCTCTCCCC,
(SEQ ID NO: 41)
CTACTGTACATCACGCCTCTCCCC,
(SEQ ID NO: 42)
GTACAGTTCCCGTCCCTGCACTACA,
or
(SEQ ID NO: 43)
GTACAGTTCCCGTCCTGCACTACA.

The MEK inhibitor in the combination therapies of the invention is binimetinib or pharmaceutically acceptable salt thereof. Binimetinib has the following structure:

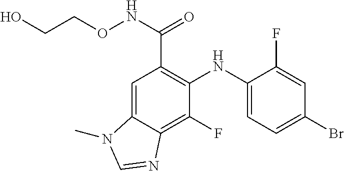 [SMILES: Cn1cnc2c(F)c(Nc3ccc(Br)cc3F)c(C(=O)NOCCO)cc21]

Binimetinib is also known as ARRY-162, MEK162, 6-(4-bromo-2-fluorophenylamino)-7-fluoro-3-methyl-3H-benzoimidazole-5-carboxylic acid (2-hydroxyethoxy)-amide, and 5-((4-bromo-2-fluorophenyl)amino)-4-fluoro-N-(2-hydroxyethoxy)-1-methyl-1H-benzimidazole-6-carboxamide. Methods of preparing binimetinib and its pharmaceutically acceptable salts are described in PCT publication No. WO 03/077914, in Example 18 (compound 29111), the disclosure of which is herein incorporated by reference in its entirety. In one embodiment, the MEK inhibitor is binimetinib as the free base. In one embodiment, the MEK inhibitor is a pharmaceutically acceptable salt of binimetinib. In one embodiment, the MEK inhibitor is crystallized binimetinib. Crystallized binimetinib and methods of preparing crystallized binimetinib are described in PCT publication No. WO 2014/063024, the disclosure of which is herein incorporated by reference in its entirety.

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer including metastatic non-small cell lung cancer), glioma, Hodgkin's lymphoma, non-Hodgkin's lymphoma, acute myeloid leukemia (AML), multiple myeloma, gastrointestinal (tract) cancer, renal cell carcinoma, renal cancer (including advanced renal cell carcinoma), ovarian cancer, liver cancer, lymphoblastic leukemia, lymphocytic leukemia, colorectal cancer (including metastatic colorectal cancer, such as microsatellite stable metastatic colorectal cancer), endometrial cancer, kidney cancer, prostate cancer, thyroid cancer, melanoma (including unresectable or metastatic melanoma, including BRAF V600 mutant melanoma, such as BRAF V600E mutant melanoma), chondrosarcoma, neuroblastoma, pancreatic cancer, glioblastoma multiforme, cervical cancer, brain cancer, stomach cancer, urothelial carcinoma (including local advanced or metastatic urothelial carcinoma), bladder cancer, hepatoma, breast cancer, colon carcinoma, and head and neck cancer (including recurrent or metastatic squamous cell carcinoma of the head and neck). In one embodiment, the cancer is colorectal cancer. In one embodiment, the cancer is metastatic colorectal cancer. In one embodiment, the cancer is microsatellite stable metastatic colorectal cancer. In one embodiment, the cancer is melanoma. In one embodiment, the cancer is pancreatic cancer. In one embodiment, the cancer is thyroid cancer.

Worldwide, colorectal cancer is the third most common type of cancer in men and the second most common in women, with approximately 1.4 million new diagnoses in 2012. Globally in 2012, approximately 694,000 deaths were attributed to colorectal cancer. The incidence of microsatellite stability (MSS) in colorectal tumors varies by stage, with nearly 80% of early stage, resectable tumors and approximately 67% of advanced, metastatic tumors exhibiting MSS.

The term “combination therapy” as used herein refers to a dosing regimen of two different therapeutically active agents (i.e., the components or combination partners of the combination) (e.g., binimetinib or a pharmaceutically acceptable salt thereof and a PD-1 binding antagonist) during a period of time, wherein the therapeutically active agents are administered together or separately in a manner prescribed by a medical care taker or according to a regulatory agency as defined herein. In one embodiment, a combination therapy consists essentially of a combination of a MEK inhibitor which is binimetinib or a pharmaceutically acceptable salt thereof, and a PD-1 binding antagonist which is nivolumab. In one embodiment, a combination therapy consists essentially of a combination of a MEK inhibitor which is binimetinib or a pharmaceutically acceptable salt thereof, and a PD-1 binding antagonist which is pembrolizumab.

As can be appreciated in the art, a combination therapy can be administered to a patient for a period of time. In some embodiments, the period of time occurs following the administration of a different cancer therapeutic treatment/agent or a different combination of cancer therapeutic treatments/agents to the patient. In some embodiments, the period of time occurs before the administration of a different cancer therapeutic treatment/agent or a different combination of cancer therapeutic treatments/agents to the patient. In some embodiments, administration of the PD-1 binding antagonist and administration of binimetinib or a pharmaceutically acceptable salt thereof occurs at substantially the same time. In some embodiments, administration of the PD-1 binding antagonist to the patient occurs prior to administration of binimetinib or a pharmaceutically acceptable salt thereof to the patient, during the period of time. In some embodiments, administration of binimetinib or a pharmaceutically acceptable salt thereof to the patient occurs prior to administration of the PD-1 binding antagonist to the patient, during the period of time. In some embodiments, the patient is administered a surgical treatment (e.g., tumor resection and/or lymph node resection) and/or anticancer therapy (e.g., an agent that does not include a BRAF kinase inhibitor, e.g., encorafenib) during the period of time. In some embodiments, the patient is not administered a BRAF kinase inhibitor (e.g., encorafenib) during the period of time.

A suitable period of time can be determined by one skilled in the art (e.g., a physician). As can be appreciated in the art, a suitable period of time can be determined by one skilled in the art based on one or more of: the stage of disease in the patient, the mass and sex of the patient, clinical trial guidelines (e.g., those on the fda.gov website), and information on the approved drug label. For example a suitable period of time can be, e.g., from 1 week to 2 years, 1 week to 22 months, 1 week to 20 months, 1 week to 18 months, 1 week to 16 months, 1 week to 14 months, 1 week to 12 months, 1 week to 10 months, 1 week to 8 months, 1 week to 6 months, 1 week to 4 months 1 week to 2 months, 1 week to 1 month, 2 weeks to 2 years, 2 weeks to 22 months, 2 weeks to 20 months, 2 weeks to 18 months, 2 weeks to 16 months, 2 weeks to 14 months, 2 weeks to 12 months, 2 weeks to 10 months, 2 weeks to 8 months, 2 weeks to 6 months, 2 weeks to 4 months, 2 weeks to 2 months, 2 weeks to 1 month, 1 month to 2 years, 1 month to 22 months, 1 month to 20 months, 1 month to 18 months, 1 month to 16 months, 1 month to 14 months, 1 month to 12 months, 1 month to 10 months, 1 month to 8 months, 1 month to 6 months, 1 month to 4 months, 1 month to 2 months, 2 months to 2 years, 2 months to 22 months, 2 months to 20 months, 2 months to 18 months, 2 months to 16 months, 2 months to 14 months, 2 months to 12 months, 2 months to 10 months, 2 months to 8 months, 2 months to 6 months, 2 months to 4 months, 3 months to 2 years, 3 months to 22 months, 3 months to 20 months, 3 months to 18 months, 3 months to 16 months, 3 months to 14 months, 3 months to 12 months, 3 months to 10 months, 3 months to 8 months, 3 months to 6 months, 4 months to 2 years, 4 months to 22 months, 4 months to 20 months, 4 months to 18 months, 4 months to 16 months, 4 months to 14 months, 4 months to 12 months, 4 months to 10 months, 4 months to 8 months, 4 months to 6 months, 6 months to 2 years, 6 months to 22 months, 6 months to 20 months, 6 months to 18 months, 6 months to 16 months, 6 months to 14 months, 6 months to 12 months, 6 months to 10 months, 6 months to 8 months, 8 months to 2 years, 8 months to 22 months, 8 months to 20 months, 8 months to 18 months, 8 months to 16 months, 8 months to 14 months, 8 months to 12 months, 8 months to 10 months, 10 months to 2 years, 10 months to 22 months, 10 months to 20 months, 10 months to 18 months, 10 months to 16 months, 10 months to 14 months, 10 months to 12 months, 12 months to 2 years, 12 months to 22 months, 12 months to 20 months, 12 months to 18 months, 12 months to 16 months, or 12 months to 14 months, inclusive.

As used herein, an “effective dosage” or “effective amount” or “therapeutically effective amount” of a drug, compound, or pharmaceutical composition is an amount sufficient to effect any one or more beneficial or desired results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing incidence or amelioration of one or more symptoms of various diseases or conditions (such as for example cancer), decreasing the dose of other medications required to treat the disease, enhancing the effect of another medication, and/or delaying the progression of the disease. An effective dosage can be administered in one or more administrations. For purposes of this invention, an effective dosage of a drug, compound, or pharmaceutical composition is an amount sufficient to accomplish prophylactic or therapeutic treatment either directly or indirectly. As is understood in the clinical context, an effective dosage of a drug, compound, or pharmaceutical composition may be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved. In reference to the treatment of cancer, an effective amount may also refer to that amount which has the effect of (1) reducing the size of the tumor, (2) inhibiting (that is, slowing to some extent, preferably stopping) tumor metastasis emergence, (3) inhibiting to some extent (that is, slowing to some extent, preferably stopping) tumor growth or tumor invasiveness, and/or (4) relieving to some extent (or, preferably, eliminating) one or more signs or symptoms associated with the cancer. Therapeutic or pharmacological effectiveness of the doses and administration regimens may also be characterized as the ability to induce, enhance, maintain or prolong disease control and/or overall survival in patients with these specific tumors, which may be measured as prolongation of the time before disease progression

The term “Q2W” as used herein means once every two weeks.

The term “Q3W” as used herein means once every three weeks.

The term “BID” as used herein means twice a day.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

The term “advanced”, as used herein, as it relates to solid tumors, includes locally advanced (non-metastatic) disease and metastatic disease. Locally advanced solid tumors, which may or may not be treated with curative intent, and metastatic disease, which cannot be treated with curative intent are included within the scope of “advanced solid tumors, as used in the present invention. Those skilled in the art will be able to recognize and diagnose advanced solid tumors in a patient.

“Tumor burden” also referred to as “tumor load”, refers to the total amount of tumor material distributed throughout the body. Tumor burden refers to the total number of cancer cells or the total size of tumor(s), throughout the body, including lymph nodes and bone narrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

“Individual response” or “response” can be assessed using any endpoint indicating a benefit to the individual, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues; (4) inhibition (i.e. reduction, slowing down, or complete stopping) of metastasis; (5) relief, to some extent, of one or more symptoms associated with the disease or disorder (e.g., cancer); (6) increase or extension in the length of survival, including overall survival and progression free survival; and/or (7) decreased mortality at a given point of time following treatment.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and/or progression-free survival); resulting in an objective response (including a complete response or a partial response); or improving signs or symptoms of cancer.

An “objective response” or “OR” refers to a measurable response, including complete response (CR) or partial response (PR). An “objective response rate” (ORR) refers to the proportion of patients with tumor size reduction of a predefined amount and for a minimum time period. Generally, ORR refers to the sum of complete response (CR) rate and partial response (PR) rate.

“Complete response” or “CR” as used herein means the disappearance of all signs of cancer (e.g., disappearance of all target lesions) in response to treatment. This does not always mean the cancer has been cured.

As used herein, “partial response” or “PR” refers to a decrease in the size of one or more tumors or lesions, or in the extent of cancer in the body, in response to treatment. For example, in some embodiments, PR refers to at least a 30% decrease in the sum of the longest diameters (SLD) of target lesions, taking as reference the baseline SLD.

“Sustained response” refers to the sustained effect on reducing tumor growth after cessation of a treatment. For example, the tumor size may be the same size or smaller as compared to the size at the beginning of the medicament administration phase. In some embodiments, the sustained response has a duration of at least the same as the treatment duration, at least 1.5×, 2×, 2.5×, or 3× length of the treatment duration, or longer.

As used herein, “progression-free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall survival” (OS) refers to the percentage of individuals in a group who are likely to be alive after a particular duration of time.

“Duration of Response” for purposes of the present invention means the time from documentation of tumor model growth inhibition due to drug treatment to the time of acquisition of a restored growth rate similar to pretreatment growth rate.

By “extending survival” is meant increasing overall or progression-free survival in a treated patient relative to an untreated patient (i.e. relative to a patient not treated with the medicament).

As used herein, “drug related toxicity”, “infusion related reactions” and “immune related adverse events” (“irAE”), and the severity or grades thereof are as exemplified and defined in the National Cancer Institute's Common Terminology Criteria for Adverse Events v 4.0 (NCI CTCAE v 4.0).

“Loss of heterozygosity score” or “LOH score” as used here in, refers to the percentage of genomic LOH in the tumor tissues of an individual. Percentage genomic LOH, and the calculation thereof are described in Swisher et al (The Lancet Oncology, 18(1):75-87, January 2017), the disclosure of which is incorporated herein by reference in its entirety. Exemplary genetic analysis includes, without limitation, DNA sequencing, and Foundation Medicine's NGS-based T5 assay.

“Homologous recombination deficiency score” or “HRD score” as used here in, refers to the unweighted numeric sum of loss of heterozygosity (“LOH”), telomeric allelic imbalance (“TAI”) and large-scale state transitions (“LST”) in the tumor tissues of an individual. HRD score, together with LOH, and LOH score, and the calculation thereof are described in Timms et al, Breast Cancer Res 2014 Dec. 5; 16(6):475, Telli et al Clin Cancer Res; 22(15); 3764-73.2016, the disclosures of which are incorporated herein by reference in their entireties. Exemplary genetic analysis includes, without limitation, DNA sequencing, Myriad's HRD or HRD Plus assay (Mirza et al N Engl J Med 2016 Dec. 1; 375(22):2154-2164, 2016).

The term “tumor proportion score” or “TPS” as used herein refers to the percentage of viable tumor cells showing partial or complete membrane staining in an immunohistochemistry test of a sample. “Tumor proportion score of PD-L1 expression” as used here in refers to the percentage of viable tumor cells showing partial or complete membrane staining in a PD-L1 expression immunohistochemistry test of a sample. Exemplary samples include, without limitation, a biological sample, a tissue sample, a formalin-fixed paraffin-embedded (FFPE) human tissue sample and a formalin-fixed paraffin-embedded (FFPE) human tumor tissue sample. Exemplary PD-L1 expression immunohistochemistry tests include, without limitation, the PD-L1 IHC 22C3 PharmDx (FDA approved, Daco), Ventana PD-L1 SP263 assay, and the tests described in international patent application PCT/EP2017/073712.

In some embodiments, the anti-cancer effects of the methods described herein, including, but not limited to “objective response”, “complete response”, “partial response”, “progressive disease”, “stable disease”, “progression free survival”, “duration of response”, are as defined and assessed by the investigators using RECIST v1.1 (Eisenhauer et al, Eur J of Cancer 2009; 45(2):228-47) in patients with locally advanced or metastatic solid tumors other than metastatic castration-resistant prostate cancer (CRPC), and RECIST v1.1 and PCWG3 (Scher et al, J Clin Oncol 2016 Apr. 20; 34(12):1402-18) in patients with metastatic CRPC. The disclosures of Eisenhauer et al, Eur J of Cancer 2009; 45(2):228-47 and Scher et al, J Clin Oncol 2016 Apr. 20; 34(12):1402-18 are herein incorporated by references in their entireties.

In some embodiments, the anti-cancer effect of the methods described herein, including, but not limited to “immune-related objective response” (irOR), “immune-related complete response” (irCR), “immune-related partial response” (irCR), “immune-related progressive disease” (irPD), “immune-related stable disease” (irSD), “immune-related progression free survival” (irPFS), “immune-related duration of response” (irDR), are as defined and assessed by Immune-related response criteria (irRECIST, Nishino et. al. J Immunother Cancer 2014; 2:17) for patients with locally advanced or metastatic solid tumors other than patients with metastatic CRPC. The disclosure of Nishino et. al. J Immunother Cancer 2014; 2:17 is herein incorporated by reference in its entirety.

As used herein, “in combination with” refers to the administration of the MEK inhibitor, which is binimetinib or a pharmaceutically acceptable salt thereof, and a PD-1 binding antagonist, concurrently, sequentially or intermittently as separate dosage.

The term “additive” is used to mean that the result of the combination of two components of the combination therapy is no greater than the sum of each compound, component or targeted agent individually. The term “additive” means that there is no improvement in the disease condition or disorder being treated over the use of each component individually.

The term “synergy” or “synergistic” is used herein to mean that the effect of the combination of the two therapeutic agents of the combination therapy is greater than the sum of the effect of each agent when administered alone. A “synergistic amount” or “synergistically effective amount” is an amount of the combination of the two combination partners that results in a synergistic effect, as “synergistic” is defined herein. Determining a synergistic interaction between two combination partners, the optimum range for the effect and absolute dose ranges of each component for the effect may be definitively measured by administration of the combination partners over different w/w (weight per weight) ratio ranges and doses to patients in need of treatment. However, the observation of synergy in in vitro models or in vivo models can be predictive of the effect in humans and other species and in vitro models or in vivo models exist, as described herein, to measure a synergistic effect and the results of such studies can also be used to predict effective dose and plasma concentration ratio ranges and the absolute doses and plasma concentrations required in humans and other species by the application of pharmacokinetic/pharmacodynamic methods. For example, art-accepted in vitro and animal models of cancers described herein are known in the art, and are described in the Examples. Exemplary synergistic effects includes, but are not limited to, enhanced therapeutic efficacy, decreased dosage at equal or increased level of efficacy, reduced or delayed development of drug resistance, and simultaneous enhancement or equal therapeutic actions and reduction of unwanted side effects.

For example, a synergistic ratio of two therapeutic agents can be identified by determining a synergistic effect in an art-accepted in vitro (e.g., cancer cell line) or in vivo (animal model) model of any of the cancers described herein. Non-limiting examples of cancer cell lines and in vivo animal models of the cancers described herein are described in the Examples. Additional examples of art-accepted cancer cell lines and in vivo animal models are known in the art.

In some embodiments, “synergistic effect” as used herein refers to combination of a MEK inhibitor which is binimetinib or a pharmaceutically acceptable salt thereof, and a PD-1 binding antagonist producing an effect, for example, any of the beneficial or desired results including clinical results as described herein, for example slowing the symptomatic progression of a proliferative disease, particularly cancer, or symptoms thereof, which is greater than the sum of effect observed when the MEK inhibitor and the PD-1 binding antagonist are administered alone.

In some embodiments, the methods provided herein can result in a 1% to 99% (e.g., 1% to 98%, 1% to 95%, 1% to 90%, 1 to 85%, 1 to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 2% to 99%, 2% to 90%, 2% to 85%, 2% to 80%, 2% to 75%, 2% to 70%, 2% to 65%, 2% to 60%, 2% to 55%, 2% to 50%, 2% to 45%, 2% to 40%, 2% to 35%, 2% to 30%, 2% to 25%, 2% to 20%, 2% to 15%, 2% to 10%, 2% to 5%, 4% to 99%, 4% to 95%, 4% to 90%, 4% to 85%, 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10%, 6% to 99%, 6% to 95%, 6% to 90%, 6% to 85%, 6% to 80%, 6% to 75%, 6% to 70%, 6% to 65%, 6% to 60%, 6% to 55%, 6% to 50%, 6% to 45%, 6% to 40%, 6% to 35%, 6% to 30%, 6% to 25%, 6% to 20%, 6% to 15%, 6% to 10%, 8% to 99%, 8% to 95%, 8% to 90%, 8% to 85%, 8% to 80%, 8% to 75%, 8% to 70%, 8% to 65%, 8% to 60%, 8% to 55%, 8% to 50%, 8% to 45%, 8% to 40%, 8% to 35%, 8% to 30%, 8% to 25%, 8% to 20%, 8% to 15%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 99%, 15% to 95%, 15% to 90%, 15% to 85%, 15% to 80%, 15% to 75%, 15% to 70%, 15% to 65%, 15% to 60%, 15% to 55%, 15% to 50%, 15% to 55%, 15% to 50%, 15% to 45%, 15% to 40%, 15% to 35%, 15% to 30%, 15% to 25%, 15% to 20%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 99%, 25% to 95%, 25% to 90%, 25% to 85%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 99%, 30% to 95%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 35% to 99%, 35% to 95%, 35% to 90%, 35% to 85%, 35% to 80%, 35% to 75%, 35% to 70%, 35% to 65%, 35% to 60%, 35% to 55%, 35% to 50%, 35% to 45%, 35% to 40%, 40% to 99%, 40% to 95%, 40% to 90%, 40% to 85%, 40% to 80%, 40% to 75%, 40% to 70%, 40% to 65%, 40% to 60%, 40% to 55%, 40% to 60%, 40% to 55%, 40% to 50%, 40% to 45%, 45% to 99%, 45% to 95%, 45% to 95%, 45% to 90%, 45% to 85%, 45% to 80%, 45% to 75%, 45% to 70%, 45% to 65%, 45% to 60%, 45% to 55%, 45% to 50%, 50% to 99%, 50% to 95%, 50% to 90%, 50% to 85%, 50% to 80%, 50% to 75%, 50% to 70%, 50% to 65%, 50% to 60%, 50% to 55%, 55% to 99%, 55% to 95%, 55% to 90%, 55% to 85%, 55% to 80%, 55% to 75%, 55% to 70%, 55% to 65%, 55% to 60%, 60% to 99%, 60% to 95%, 60% to 90%, 60% to 85%, 60% to 80%, 60% to 75%, 60% to 70%, 60% to 65%, 65% to 99%, 60% to 95%, 60% to 90%, 60% to 85%, 60% to 80%, 60% to 75%, 60% to 70%, 60% to 65%, 70% to 99%, 70% to 95%, 70% to 90%, 70% to 85%, 70% to 80%, 70% to 75%, 75% to 99%, 75% to 95%, 75% to 90%, 75% to 85%, 75% to 80%, 80% to 99%, 80% to 95%, 80% to 90%, 80% to 85%, 85% to 99%, 85% to 95%, 85% to 90%, 90% to 99%, 90% to 95%, or 95% to 100%) reduction in the volume of one or more solid tumors in a patient following treatment with the combination therapy for a period of time between 1 day and 2 years (e.g., between 1 day and 22 months, between 1 day and 20 months, between 1 day and 18 months, between 1 day and 16 months, between 1 day and 14 months, between 1 day and 12 months, between 1 day and 10 months, between 1 day and 9 months, between 1 day and 8 months, between 1 day and 7 months, between 1 day and 6 months, between 1 day and 5 months, between 1 day and 4 months, between 1 day and 3 months, between 1 day and 2 months, between 1 day and 1 month, between one week and 2 years, between 1 week and 22 months, between 1 week and 20 months, between 1 week and 18 months, between 1 week and 16 months, between 1 week and 14 months, between 1 week and 12 months, between 1 week and 10 months, between 1 week and 9 months, between 1 week and 8 months, between 1 week and 7 months, between 1 week and 6 months, between 1 week and 5 months, between 1 week and 4 months, between 1 week and 3 months, between 1 week and 2 months, between 1 week and 1 month, between 2 weeks and 2 years, between 2 weeks and 22 months, between 2 weeks and 20 months, between 2 weeks and 18 months, between 2 weeks and 16 months, between 2 weeks and 14 months, between 2 weeks and 12 months, between 2 weeks and 10 months, between 2 weeks and 9 months, between 2 weeks and 8 months, between 2 weeks and 7 months, between 2 weeks and 6 months, between 2 weeks and 5 months, between 2 weeks and 4 months, between 2 weeks and 3 months, between 2 weeks and 2 months, between 2 weeks and 1 month, between 1 month and 2 years, between 1 month and 22 months, between 1 month and 20 months, between 1 month and 18 months, between 1 month and 16 months, between 1 month and 14 months, between 1 month and 12 months, between 1 month and 10 months, between 1 month and 9 months, between 1 month and 8 months, between 1 month and 7 months, between 1 month and 6 months, between 1 month and 6 months, between 1 month and 5 months, between 1 month and 4 months, between 1 month and 3 months, between 1 month and 2 months, between 2 months and 2 years, between 2 months and 22 months, between 2 months and 20 months, between 2 months and 18 months, between 2 months and 16 months, between 2 months and 14 months, between 2 months and 12 months, between 2 months and 10 months, between 2 months and 9 months, between 2 months and 8 months, between 2 months and 7 months, between 2 months and 6 months, or between 2 months and 5 months, between 2 months and 4 months, between 3 months and 2 years, between 3 months and 22 months, between 3 months and 20 months, between 3 months and 18 months, between 3 months and 16 months, between 3 months and 14 months, between 3 months and 12 months, between 3 months and 10 months, between 3 months and 8 months, between 3 months and 6 months, between 4 months and 2 years, between 4 months and 22 months, between 4 months and 20 months, between 4 months and 18 months, between 4 months and 16 months, between 4 months and 14 months, between 4 months and 12 months, between 4 months and 10 months, between 4 months and 8 months, between 4 months and 6 months, between 6 months and 2 years, between 6 months and 22 months, between 6 months and 20 months, between 6 months and 18 months, between 6 months and 16 months, between 6 months and 14 months, between 6 months and 12 months, between 6 months and 10 months, or between 6 months and 8 months) (e.g., as compared to the size of the one or more solid tumors in the patient prior to treatment).

In some embodiments, any of the methods described herein can provide for a 1% to 99% (e.g., 1% to 98%, 1% to 95%, 1% to 90%, 1 to 85%, 1 to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 2% to 99%, 2% to 90%, 2% to 85%, 2% to 80%, 2% to 75%, 2% to 70%, 2% to 65%, 2% to 60%, 2% to 55%, 2% to 50%, 2% to 45%, 2% to 40%, 2% to 35%, 2% to 30%, 2% to 25%, 2% to 20%, 2% to 15%, 2% to 10%, 2% to 5%, 4% to 99%, 4% to 95%, 4% to 90%, 4% to 85%, 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10%, 6% to 99%, 6% to 95%, 6% to 90%, 6% to 85%, 6% to 80%, 6% to 75%, 6% to 70%, 6% to 65%, 6% to 60%, 6% to 55%, 6% to 50%, 6% to 45%, 6% to 40%, 6% to 35%, 6% to 30%, 6% to 25%, 6% to 20%, 6% to 15%, 6% to 10%, 8% to 99%, 8% to 95%, 8% to 90%, 8% to 85%, 8% to 80%, 8% to 75%, 8% to 70%, 8% to 65%, 8% to 60%, 8% to 55%, 8% to 50%, 8% to 45%, 8% to 40%, 8% to 35%, 8% to 30%, 8% to 25%, 8% to 20%, 8% to 15%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 99%, 15% to 95%, 15% to 90%, 15% to 85%, 15% to 80%, 15% to 75%, 15% to 70%, 15% to 65%, 15% to 60%, 15% to 55%, 15% to 50%, 15% to 55%, 15% to 50%, 15% to 45%, 15% to 40%, 15% to 35%, 15% to 30%, 15% to 25%, 15% to 20%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 99%, 25% to 95%, 25% to 90%, 25% to 85%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 99%, 30% to 95%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 35% to 99%, 35% to 95%, 35% to 90%, 35% to 85%, 35% to 80%, 35% to 75%, 35% to 70%, 35% to 65%, 35% to 60%, 35% to 55%, 35% to 50%, 35% to 45%, 35% to 40%, 40% to 99%, 40% to 95%, 40% to 90%, 40% to 85%, 40% to 80%, 40% to 75%, 40% to 70%, 40% to 65%, 40% to 60%, 40% to 55%, 40% to 60%, 40% to 55%, 40% to 50%, 40% to 45%, 45% to 99%, 45% to 95%, 45% to 95%, 45% to 90%, 45% to 85%, 45% to 80%, 45% to 75%, 45% to 70%, 45% to 65%, 45% to 60%, 45% to 55%, 45% to 50%, 50% to 99%, 50% to 95%, 50% to 90%, 50% to 85%, 50% to 80%, 50% to 75%, 50% to 70%, 50% to 65%, 50% to 60%, 50% to 55%, 55% to 99%, 55% to 95%, 55% to 90%, 55% to 85%, 55% to 80%, 55% to 75%, 55% to 70%, 55% to 65%, 55% to 60%, 60% to 99%, 60% to 95%, 60% to 90%, 60% to 85%, 60% to 80%, 60% to 75%, 60% to 70%, 60% to 65%, 65% to 99%, 60% to 95%, 60% to 90%, 60% to 85%, 60% to 80%, 60% to 75%, 60% to 70%, 60% to 65%, 70% to 99%, 70% to 95%, 70% to 90%, 70% to 85%, 70% to 80%, 70% to 75%, 75% to 99%, 75% to 95%, 75% to 90%, 75% to 85%, 75% to 80%, 80% to 99%, 80% to 95%, 80% to 90%, 80% to 85%, 85% to 99%, 85% to 95%, 85% to 90%, 90% to 99%, 90% to 95%, or 95% to 100%) reduction in the risk of developing a metastasis or the risk of developing an additional metastasis in a patient having a cancer.

The phrase “time of survival” means the length of time between the identification or diagnosis of cancer (e.g., any of the cancers described herein) in a mammal by a medical professional and the time of death of the mammal (caused by the cancer). Methods of increasing the time of survival in a mammal having a cancer are described herein.

In some embodiments, any of the methods described herein can result in an increase (e.g., a 1% to 400%, 1% to 380%, 1% to 360%, 1% to 340%, 1% to 320%, 1% to 300%, 1% to 280%, 1% to 260%, 1% to 240%, 1% to 220%, 1% to 200%, 1% to 180%, 1% to 160%, 1% to 140%, 1% to 120%, 1% to 100%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 5% to 400%, 5% to 380%, 5% to 360%, 5% to 340%, 5% to 320%, 5% to 300%, 5% to 280%, 5% to 260%, 5% to 240%, 5% to 220%, 5% to 200%, 5% to 180%, 5% to 160%, 5% to 140%, 5% to 120%, 5% to 100%, 5% to 90%, 5% to 80%, 5% to 70%, 5% to 60%, 5% to 50%, 5% to 40%, 5% to 30%, 5% to 20%, 5% to 10%, 10% to 400%, 10% to 380%, 10% to 360%, 10% to 340%, 10% to 320%, 10% to 300%, 10% to 280%, 10% to 260%, 10% to 240%, 10% to 220%, 10% to 200%, 10% to 180%, 10% to 160%, 10% to 140%, 10% to 120%, 10% to 100%, 10% to 90%, 10% to 80%, 10% to 70%, 10% to 60%, 10% to 50%, 10% to 40%, 10% to 30%, 10% to 20%, 20% to 400%, 20% to 380%, 20% to 360%, 20% to 340%, 20% to 320%, 20% to 300%, 20% to 280%, 20% to 260%, 20% to 240%, 20% to 220%, 20% to 200%, 20% to 180%, 20% to 160%, 20% to 140%, 20% to 120%, 20% to 100%, 20% to 90%, 20% to 80%, 20% to 70%, 20% to 60%, 20% to 50%, 20% to 40%, 20% to 30%, 30% to 400%, 30% to 380%, 30% to 360%, 30% to 340%, 30% to 320%, 30% to 300%, 30% to 280%, 30% to 260%, 30% to 240%, 30% to 220%, 30% to 200%, 30% to 180%, 30% to 160%, 30% to 140%, 30% to 120%, 30% to 100%, 30% to 90%, 30% to 80%, 30% to 70%, 30% to 60%, 30% to 50%, 30% to 40%, 40% to 400%, 40% to 380%, 40% to 360%, 40% to 340%, 40% to 320%, 40% to 300%, 40% to 280%, 40% to 260%, 40% to 240%, 40% to 220%, 40% to 200%, 40% to 180%, 40% to 160%, 40% to 140%, 40% to 120%, 40% to 100%, 40% to 90%, 40% to 80%, 40% to 70%, 40% to 60%, 40% to 50%, 50% to 400%, 50% to 380%, 50% to 360%, 50% to 340%, 50% to 320%, 50% to 300%, 50% to 280%, 50% to 260%, 50% to 240%, 50% to 220%, 50% to 200%, 50% to 180%, 50% to 160%, 50% to 140%, 50% to 140%, 50% to 120%, 50% to 100%, 50% to 90%, 50% to 80%, 50% to 70%, 50% to 60%, 60% to 400%, 60% to 380%, 60% to 360%, 60% to 340%, 60% to 320%, 60% to 300%, 60% to 280%, 60% to 260%, 60% to 240%, 60% to 220%, 60% to 200%, 60% to 180%, 60% to 160%, 60% to 140%, 60% to 120%, 60% to 100%, 60% to 90%, 60% to 80%, 60% to 70%, 70% to 400%, 70% to 380%, 70% to 360%, 70% to 340%, 70% to 320%, 70% to 300%, 70% to 280%, 70% to 260%, 70% to 240%, 70% to 220%, 70% to 200%, 70% to 180%, 70% to 160%, 70% to 140%, 70% to 120%, to 100%, 70% to 90%, 70% to 80%, 80% to 400%, 80% to 380%, 80% to 360%, 80% to 340%, 80% to 320%, 80% to 300%, 80% to 280%, 80% to 260%, 80% to 240%, 80% to 220%, 80% to 200%, 80% to 180%, 80% to 160%, 80% to 140%, 80% to 120%, 80% to 100%, 80% to 90%, 90% to 400%, 90% to 380%, 90% to 360%, 90% to 340%, 90% to 320%, 90% to 300%, 90% to 280%, 90% to 260%, 90% to 240%, 90% to 220%, 90% to 200%, 90% to 180%, 90% to 160%, 90% to 140%, 90% to 120%, 90% to 100%, 100% to 400%, 100% to 380%, 100% to 360%, 100% to 340%, 100% to 320%, 100% to 300%, 100% to 280%, 100% to 260%, 100% to 240%, 100% to 220%, 100% to 200%, 100% to 180%, 100% to 160%, 100% to 140%, 100% to 120%, 120% to 400%, 120% to 380%, 120% to 360%, 120% to 340%, 120% to 320%, 120% to 300%, 120% to 280%, 120% to 260%, 120% to 240%, 120% to 220%, 120% to 200%, 120% to 180%, 120% to 160%, 120% to 140%, 140% to 400%, 140% to 380%, 140% to 360%, 140% to 340%, 140% to 320%, 140% to 300%, 140% to 280%, 140% to 260%, 140% to 240%, 140% to 220%, 140% to 200%, 140% to 180%, 140% to 160%, 160% to 400%, 160% to 380%, 160% to 360%, 160% to 340%, 160% to 320%, 160% to 300%, 160% to 280%, 160% to 260%, 160% to 240%, 160% to 220%, 160% to 200%, 160% to 180%, 180% to 400%, 180% to 380%, 180% to 360%, 180% to 340%, 180% to 320%, 180% to 300%, 180% to 280%, 180% to 260%, 180% to 240%, 180% to 220%, 180% to 200%, 200% to 400%, 200% to 380%, 200% to 360%, 200% to 340%, 200% to 320%, 200% to 300%, 200% to 280%, 200% to 260%, 200% to 240%, 200% to 220%, 220% to 400%, 220% to 380%, 220% to 360%, 220% to 340%, 220% to 320%, 220% to 300%, 220% to 280%, 220% to 260%, 220% to 240%, 240% to 400%, 240% to 380%, 240% to 360%, 240% to 340%, 240% to 320%, 240% to 300%, 240% to 280%, 240% to 260%, 260% to 400%, 260% to 380%, 260% to 360%, 260% to 340%, 260% to 320%, 260% to 300%, 260% to 280%, 280% to 400%, 280% to 380%, 280% to 360%, 280% to 340%, 280% to 320%, 280% to 300%, 300% to 400%, 300% to 380%, 300% to 360%, 300% to 340%, or 300% to 320%) in the time of survival of the patient (e.g., as compared to a patient having a similar cancer and administered a different treatment or not receiving a treatment).

As used herein, the term “cytokine” refers generically to proteins released by one cell population that act on another cell as intercellular mediators or have an autocrine effect on the cells producing the proteins. Examples of such cytokines include lymphokines, monokines; interleukins (“ILs”) such as IL-1, IL-la, IL-2, IL-3, IL-4, IL-5, IL-6, IL-7, IL-8, IL-9, IL10, IL-11, IL-12, IL-13, IL-15, IL-17A-F, IL-18 to IL-29 (such as IL-23), IL-31, including PROLEUKIN® rIL-2; a tumor-necrosis factor such as TNF-α or TNF-β, TGF-I-3; and other polypeptide factors including leukemia inhibitory factor (“LIF”), ciliary neurotrophic factor (“CNTF”), CNTF-like cytokine (“CLC”), cardiotrophin (“CT”), and kit ligand (“L”).

As used herein, the term “chemokine” refers to soluble factors (e.g., cytokines) that have the ability to selectively induce chemotaxis and activation of leukocytes. They also trigger processes of angiogenesis, inflammation, wound healing, and tumorigenesis. Example chemokines include IL-8, a human homolog of murine keratinocyte chemoattractant (KC).

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. As used herein, the singular form “a”, “an”, and “the” include plural references unless indicated otherwise. For example, “an” excipient includes one or more excipients. It is understood that aspects and variations of the invention described herein include “consisting of” and/or “consisting essentially of” aspects and variations. In some embodiments, methods consisting essentially of an administration step as disclosed herein include methods wherein a patient has failed a prior therapy (administered to the patient before the period of time) or has been refractory to such prior therapy, and/or wherein the cancer has metastasized or recurred. In some embodiments, methods consisting essentially of an administration step as disclosed herein include methods wherein a patient undergoes surgery, radiation, and/or other regimens prior to, substantially at the same time as, or following such an administration step as disclosed herein, and/or where the patient is administered other chemical and/or biological therapeutic agents following such an administration step as disclosed herein.

Exemplary methods and materials are described herein, although methods and materials similar or equivalent to those described herein can also be used in the practice or testing of the invention. The materials, methods, and examples are illustrative only and not intended to be limiting.

### Methods, Uses, and Medicaments

In one embodiment, an amount of a MEK inhibitor, which is binimetinib or a pharmaceutically acceptable salt thereof, is used in combination with an amount of a PD-1 binding antagonist, wherein the amounts together are effective in the treatment of cancer.

In one embodiment, a therapeutically effective amount of each of the combination partners of a combination therapy of the invention are administered separately and may be administered simultaneously, sequentially, or intermittently, and in any order, at specific or varying time intervals (e.g., during the period of time).

In one embodiment, provided herein is a method of treating a proliferative disease, including cancer, which comprises or consists essentially of administering, during the period of time, of a combination therapy consisting essentially of a MEK inhibitor which is binimetinib or a pharmaceutically acceptable salt thereof, and a PD-1 binding antagonist, to a patient in need thereof, wherein the individual combination partners are administered in jointly therapeutically effective amounts (for example in synergistically effective amounts). The individual combination partners of a combination therapy of the invention may be administered in daily or intermittent dosages during the period of time. The individual combination partners of a combination therapy of the invention may be administered separately at different times and in any order during the period of time, or concurrently in divided combination forms during the period of time. In one embodiment, the MEK inhibitor, which is binimetinib or a pharmaceutically acceptable salt thereof, is administered on a daily basis, either once daily or twice daily, during the period of time. In one embodiment, the MEK inhibitor which is binimetinib or a pharmaceutically acceptable salt thereof is administered twice daily on a daily basis, during the period of time. In one embodiment, the PD-1 binding antagonist is administered on a weekly basis, during the period of time. In one embodiment, the PD-1 binding antagonist is administered every 2 weeks (Q2W), during the period of time. In one embodiment, the PD-1 binding antagonist is administered every 3 weeks (Q3W), during the period of time. The instant invention is therefore to be understood as embracing all such regimens of simultaneous or alternating treatment during the period of time and the term “administering” is to be interpreted accordingly.

The term “jointly therapeutically effective amount” as used herein means when the therapeutic agents of a combination described herein are given to the patient simultaneously or separately (e.g., in a chronologically staggered manner, for example a sequence-specific manner) in such time intervals that they show an interaction (e.g., a joint therapeutic effect, for example a synergistic effect). Whether this is the case can, inter alia, be determined by following the blood levels and showing that the combination components are present in the blood of the human to be treated at least during certain time intervals.

In one embodiment, provided herein is a method of treating a subject having a proliferative disease comprising, consisting essentially of, or consisting of administering to said subject a combination therapy as described herein, during a period of time, in a quantity which is jointly therapeutically effective against a proliferative disease. In one embodiment, the proliferative disease is cancer. In one embodiment, the cancer is selected from squamous cell carcinoma, myeloma, small-cell lung cancer, non-squamous non-small cell lung cancer, advanced non-small cell lung cancer, non-small cell lung cancer including metastatic non-small cell lung cancer), glioma, Hodgkin's lymphoma, non-Hodgkin's lymphoma, acute myeloid leukemia (AML), multiple myeloma, gastrointestinal (tract) cancer, renal cancer (including advanced renal cell carcinoma), ovarian cancer, liver cancer, lymphoblastic leukemia, lymphocytic leukemia, colorectal cancer (including metastatic colorectal cancer, such as microsatellite stable metastatic colorectal cancer), endometrial cancer, kidney cancer, prostate cancer, thyroid cancer, melanoma (including unresectable or metastatic melanoma, including BRAF V600 mutant melanoma), advanced melanoma, microsattelite instability-high cancer, chondrosarcoma, neuroblastoma, pancreatic cancer, glioblastoma multiforme, cervical cancer, brain cancer, stomach cancer, urothelial carcinoma (including local advanced or metastatic urothelial carcinoma), bladder cancer, hepatoma, breast cancer, colon carcinoma, head and neck squamous cell cancer, and head and neck cancer (including recurrent or metastatic squamous cell carcinoma of the head and neck). In one embodiment, the cancer is colorectal cancer. In one embodiment, the cancer is metastatic colorectal cancer. In one embodiment, the cancer is microsatellite stable metastatic colorectal cancer. In one embodiment, the cancer is melanoma. In one embodiment, the cancer is pancreatic cancer. In one embodiment, the cancer is thyroid cancer.

In one embodiment, the cancer is selected from non-small cell lung cancer, pancreatic cancer, ovarian cancer, colorectal cancer, gastric cancer, melanoma, breast cancer, bladder cancer, non-small cell lung cancer, head and neck cancer, uterine cancer, cervical cancer, liver cancer, thyroid cancer, kidney cancer, brain cancer, skin cancer, and mesothelioma.

In one embodiment, the cancer is pancreatic cancer. In one embodiment, the pancreatic cancer is MMS/MMR-proficient pancreatic ductal adenocarcinoma. In one embodiment, the cancer is ovarian cancer. In one embodiment, the cancer is colorectal cancer. In one embodiment, the cancer is metastatic colorectal cancer. In one embodiment, the cancer is gastric cancer. In one embodiment, the cancer is melanoma. In one embodiment, the cancer is advanced, unresectable or metastatic melanoma. In one embodiment, the cancer is breast cancer. In one embodiment, the cancer is triple negative breast cancer. In one embodiment, the cancer is bladder cancer. In one embodiment, the cancer is non-small cell lung cancer. In one embodiment, the cancer is advanced or metastatic PD-L1 positive non-small cell lung cancer.

In some embodiments, the subject was previously treated, before the period of time, with one or more therapeutic agents, e.g., treatment with at least one anticancer treatment independently selected from chemotherapy, targeted therapeutic agents (e.g., Keytruda, Opdivo, or binimetinib or a pharmaceutically acceptable salt thereof, as a monotherapy; or a combination of a MEK inhibitor (e.g., binimetinib or a pharmaceutically acceptable salt thereof) and a BRAF kinase inhibitor (e.g., encorafenib)), radiation therapy, and surgery.

The term “chemotherapy” or “chemotherapeutic agent” as used herein refers to a chemotherapeutic agent, or a combination of two, three, four, or more chemotherapeutic agents, for the treatment of cancer. When a chemotherapy consists more than one chemotherapeutic agents, the chemotherapeutic agents can be administered to the patient on the same day or on different days in the same treatment cycle.

A “chemotherapeutic agent” is a chemical compound useful in the treatment of cancer. Examples of chemotherapeutic agents include alkylating agents such as thiotepa and cyclophosphamide (CYTOXAN®); alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; pemetrexed; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB 1-TM1); eleutherobin; pancratistatin; TLK-286; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gamma I I and calicheamicin omegal I (see, e.g., Nicolaou et ai, Angew. Chem Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HC1 liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and fluoropyrimidine-containing chemotherapies such as 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDIS1NE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel, also known as nab-paclitaxel (ABRAXANE™), and doxetaxel (TAXOTERE®)); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; am inopterin; ibandronate; topoisomerase inhibitor RFS 2000; difluorometlhylomithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, FOLFOX, an abbreviation for a treatment regimen with oxaliplatin (ELOXATIN™) combined with 5-FU and leucovovin, and FOLFOXIRI an abbreviation for a treatment regimen with irinotecan and oxaliplatin and 5-fluorouracil.

Additional examples of chemotherapeutic agents include anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRFI) agonists such as leuprolide acetate (LUPRON® and ELIGARD®), goserelin acetate, buserelin acetate and tripterelin; anti-androgens such as fiutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RJVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); an anti-estrogen such as fulvestrant; irinotecan; rmRH (e.g., ABARELIX®); 17AAG (geldanamycin derivative that is a heat shock protein (Hsp) 90 poison), and pharmaceutically acceptable salts, acids or derivatives of any of the above.

A “platinum-based chemotherapy” as used herein, refers to a chemotherapy wherein at least one chemotherapeutic agent is a coordination complex of platinum. Exemplary platinum-based chemotherapy includes, without limitation, cisplatin, carboplatin, oxaliplatin, nedaplatin, gemcitabine in combination with cisplatin, carboplatin in combination with pemetremed.

A “targeted therapeutic agent” as used herein includes, refers to a molecule that blocks the growth of cancer cells by interfering with specific targeted molecules needed for carcinogenesis and tumor growth, rather than by simply interfering with all rapidly dividing cells (e.g. with traditional chemotherapy), and includes but is not limited to, receptor tyrosine kinase-targeted therapeutic agents (for example cabozantinib, crizotinib, erlotinib, gefitinib, imatinib, lapatinib, nilotinib, pazopanib, pertuzumab, regorafenib, sunitinib, and trastuzumab), signal transduction pathway inhibitors (for example, Ras-Raf-MEK-ERK pathway inhibitors (e.g. sorafenib, trametinib, vemurafenib), PI3K-Akt-mTOR-S6K pathway inhibitors (e.g. everolimus, rapamycin, perifosine, temsirolimus) and modulators of the apoptosis pathway (e.g. obataclax)), and angiogenesis-targeted therapies (for example, aflibercept and bevacizumab). In one embodiment, the one or more therapeutic agents that were administered to the patient before the period of time is chemotherapy. In one embodiment, chemotherapy is selected from one or more of a platinum-based chemotherapy and a fluoropyrimidine-containing therapy. In one embodiment, the one or more therapeutic agents that were administered to the patient before the period of time is a platinum-based chemotherapy. In one embodiment, the one or more therapeutic agents that were administered to the patient before the period of time is a fluoropyrimidine-containing chemotherapy (e.g., fluorouracil (5-FU)). In one embodiment, the one or more therapeutic agents that were administered to the patient before the period of time is FOLFIRINOX (a chemotherapy treatment regimen of folinic acid (leucovorin), fluorouracil (5-FU), irinotecan, and oxaliplatin). In one embodiment, the one or more therapeutic agents that were administered to the patient before the period of time is FOLFOXIRI (a chemotherapy regimen of irinotecan and oxaliplatin plus 5-fluorouracil). In one embodiment, the cancer has progressed after prior treatment with a platinum-based chemotherapy.

In some embodiments of any of the methods described herein, the one or more therapeutic agents that were administered to the patient before the period of time was unsuccessful (e.g., therapeutically unsuccessful as determined by a physician).

In one embodiment, the one or more therapeutic agents that were administered to the patient before the period of time includes an angiogenesis-targeted agent.

In one embodiment, the patient has been administered surgery before the period of time. Non-limiting examples of surgery include, e.g., open surgery or minimally invasive surgery. Surgery can include, e.g., removing an entire tumor, debulking of a tumor, or removing a tumor that is causing pain or pressure in the subject. Methods for performing open surgery and minimally invasive surgery on a subject having a cancer are known in the art.

In one embodiment, the patient has received radiotherapy before the period of time. Non-limiting examples of radiation therapy include external radiation beam therapy (e.g., external beam therapy using kilovoltage X-rays or megavoltage X-rays) or internal radiation therapy. Internal radiation therapy (also called brachytherapy) can include the use of, e.g., low-dose internal radiation therapy or high-dose internal radiation therapy. Low-dose internal radiation therapy includes, e.g., inserting small radioactive pellets (also called seeds) into or proximal to a cancer tissue in the subject. High-dose internal radiation therapy includes, e.g., inserting a thin tube (e.g., a catheter) or an implant into or proximal to a cancer tissue in the subject, and delivering a high dose of radiation to the thin tube or implant using a radiation machine. Methods for performing radiation therapy on a subject having a cancer are known in the art.

In one embodiment, the MEK inhibitor is binimetinib as the free base. In one embodiment, the MEK inhibitor is a pharmaceutically acceptable salt of binimetinib. In one embodiment, the MEK inhibitor is crystallized binimetinib. In one embodiment, binimetinib is orally administered during the period of time. In one embodiment, binimetinib is administered as a tablet during the period of time. In one embodiment, a tablet formulation of binimetinib comprises about 5 mg to about 50 mg (e.g., 5 mg to about 45 mg, about 5 mg to about 40 mg, about 5 mg to about 35 mg, about 5 mg to about 30 mg, about 5 mg to about 25 mg, about 5 mg to about 20 mg, about 5 mg to about 18 mg, about 5 mg to about 16 mg, about 5 mg to about 14 mg, about 5 mg to about 12 mg, about 5 mg to about 10 mg, about 5 mg to about 8 mg, about 10 mg to about 50 mg, about 10 mg to about 45 mg, about 10 mg to about 40 mg, about 10 mg to about 35 mg, about 10 mg to about 30 mg, about 10 mg to about 25 mg, about 10 mg to about 20 mg, about 10 mg to about 18 mg, about 10 mg to about 16 mg, about 10 mg to about 14 mg, about 10 mg to about 12 mg, about 12 mg to about 50 mg, about 12 mg to about 45 mg, about 12 mg to about 45 mg, about 12 mg to about 40 mg, about 12 mg to about 35 mg, about 12 mg to about 30 mg, about 12 mg to about 25 mg, about 12 mg to about 20 mg, about 12 mg to about 18 mg, about 12 mg to about 16 mg, about 12 mg to about 14 mg, about 14 mg to about 50 mg, about 14 mg to about 45 mg, about 14 mg to about 40 mg, about 14 mg to about 35 mg, about 14 mg to about 30 mg, about 14 mg to about 25 mg, about 14 mg to about 20 mg, about 14 mg to about 18 mg, about 14 mg to about 16 mg, about 16 mg to about 50 mg, about 16 mg to about 45 mg, about 16 mg to about 40 mg, about 16 mg to about 35 mg, about 16 mg to about 30 mg, about 16 mg to about 25 mg, about 16 mg to about 20 mg, about 16 mg to about 18 mg, about 18 mg to about 50 mg, about 18 mg to about 45 mg, about 18 mg to about 40 mg, about 18 mg to about 35 mg, about 18 mg to about 30 mg, about 18 mg to about 25 mg, about 18 mg to about 20 mg, about 20 mg to about 50 mg, about 20 mg to about 45 mg, about 20 mg to about 40 mg, about 20 mg to about 35 mg, about 20 mg to about 30 mg, about 20 mg to about 25 mg, about 25 mg to about 50 mg, about 25 mg to about 45 mg, about 25 mg to about 40 mg, about 25 mg to about 35 mg, about 25 mg to about 30 mg, about 30 mg to about 50 mg, about 30 mg to about 45 mg, about 30 mg to about 40 mg, about 30 mg to about 35 mg, about 35 mg to about 50 mg, about 35 mg to about 45 mg, about 35 mg to about 40 mg, about 40 mg to about 50 mg, about 40 mg to about 45 mg, about 45 mg to about 50 mg, about 5 mg, about 10 mg, about 15 mg, about 20 mg, about 25 mg, about 30 mg, about 35 mg, about 40 mg, about 45 mg, or about 50 mg) of binimetinib or a pharmaceutically acceptable salt thereof. In one embodiment, a tablet formulation of binimetinib comprises about 5 mg to about 50 mg (e.g., any of the subranges or values within this range described herein, e.g., about 15 mg) of crystallized binimetinib. In one embodiment, binimetinib is orally administered twice daily during the period of time. In one embodiment, binimetinib is orally administered twice daily during the period of time, wherein the second dose of binimetinib is administered about 1 hour, about 2 hours, about 3 hours, about 4 hours, about 5 hours, about 6 hours, about 7 hours, about 8 hours, about 9 hours, about 10 hours, about 11 hours, or about 12 hours (e.g., 12 hours±2 hours) after the first dose of binimetinib during the period of time. In one embodiment, binimetinib is orally administered daily in the amount of about 10 mg to about 100 mg (e.g., about 10 mg to about 95 mg, about 10 mg to about 90 mg, about 10 mg to about 85 mg, about 10 mg to about 80 mg, about 10 mg to about 75 mg, about 10 mg to about 70 mg, about 10 mg to about 65 mg, about 10 mg to about 60 mg, about 10 mg to about 55 mg, about 10 mg to about 50 mg, about 10 mg to about 45 mg, about 10 mg to about 40 mg, about 10 mg to about 35 mg, about 10 mg to about 30 mg, about 10 mg to about 25 mg, about 10 mg to about 20 mg, about 10 mg to about 15 mg, about 15 mg to about 100 mg, about 15 mg to about 95 mg, about 15 mg to about 90 mg, about 15 mg to about 85 mg, about 15 mg to about 80 mg, about 15 mg to about 75 mg, about 15 mg to about 70 mg, about 15 mg to about 65 mg, about 15 mg to about 60 mg, about 15 mg to about 55 mg, about 15 mg to about 50 mg, about 15 mg to about 45 mg, about 15 mg to about 40 mg, about 15 mg to about 35 mg, about 15 mg to about 30 mg, about 15 mg to about 25 mg, about 15 mg to about 20 mg, about 20 mg to about 100 mg, about 20 mg to about 95 mg, about 20 mg to about 90 mg, about 20 mg to about 85 mg, about 20 mg to about 80 mg, about 20 mg to about 75 mg, about 20 mg to about 70 mg, about 20 mg to about 65 mg, about 20 mg to about 60 mg, about 20 mg to about 55 mg, about 20 mg to about 50 mg, about 20 mg to about 45 mg, about 20 mg to about 40 mg, about 20 mg to about 35 mg, about 20 mg to about 30 mg, about 20 mg to about 25 mg, about 25 mg to about 100 mg, about 25 mg to about 95 mg, about 25 mg to about 90 mg, about 25 mg to about 85 mg, about 25 mg to about 80 mg, about 25 mg to about 75 mg, about 25 mg to about 70 mg, about 25 mg to about 65 mg, about 25 mg to about 60 mg, about 25 mg to about 55 mg, about 25 mg to about 50 mg, about 25 mg to about 45 mg, about 25 mg to about 40 mg, about 25 mg to about 35 mg, about 25 mg to about 30 mg, about 30 mg to about 100 mg, about 30 mg to about 95 mg, about 30 mg to about 90 mg, about 30 mg to about 85 mg, about 30 mg to about 80 mg, about 30 mg to about 75 mg, about 30 mg to about 70 mg, about 30 mg to about 65 mg, about 30 mg to about 60 mg, about 30 mg to about 55 mg, about 30 mg to about 50 mg, about 30 mg to about 45 mg, about 30 mg to about 40 mg, about 30 mg to about 35 mg, about 35 mg to about 100 mg, about 35 mg to about 95 mg, about 35 mg to about 90 mg, about 35 mg to about 85 mg, about 35 mg to about 80 mg, about 35 mg to about 75 mg, about 35 mg to about 70 mg, about 35 mg to about 65 mg, about 35 mg to about 60 mg, about 35 mg to about 55 mg, about 35 mg to about 50 mg, about 35 mg to about 45 mg, about 35 mg to about 40 mg, about 40 mg to about 100 mg, about 40 mg to about 95 mg, about 40 mg to about 90 mg, about 40 mg to about 85 mg, about 40 mg to about 80 mg, about 40 mg to about 75 mg, about 40 mg to about 70 mg, about 40 mg to about 65 mg, about 40 mg to about 60 mg, about 40 mg to about 55 mg, about 40 mg to about 50 mg, about 40 mg to about 45 mg, about 45 mg to about 100 mg, about 45 mg to about 95 mg, about 45 mg to about 90 mg, about 45 mg to about 85 mg, about 45 mg to about 80 mg, about 45 mg to about 75 mg, about 45 mg to about 70 mg, about 45 mg to about 65 mg, about 45 mg to about 60 mg, about 45 mg to about 55 mg, about 45 mg to about 50 mg, about 50 mg to about 100 mg, about 50 mg to about 95 mg, about 50 mg to about 90 mg, about 50 mg to about 85 mg, about 50 mg to about 80 mg, about 50 mg to about 75 mg, about 50 mg to about 70 mg, about 50 mg to about 65 mg, about 50 mg to about 60 mg, about 50 mg to about 55 mg, about 55 mg to about 100 mg, about 55 mg to about 95 mg, about 55 mg to about 90 mg, about 55 mg to about 85 mg, about 55 mg to about 80 mg, about 55 mg to about 75 mg, about 55 mg to about 70 mg, about 55 mg to about 65 mg, about 55 mg to about 60 mg, about 60 mg to about 100 mg, about 60 mg to about 95 mg, about 60 mg to about 90 mg, about 60 mg to about 85 mg, about 60 mg to about 80 mg, about 60 mg to about 75 mg, about 60 mg to about 70 mg, about 60 mg to about 65 mg, about 65 mg to about 100 mg, about 65 mg to about 95 mg, about 65 mg to about 90 mg, about 65 mg to about 85 mg, about 65 mg to about 80 mg, about 65 mg to about 75 mg, about 65 mg to about 70 mg, about 70 mg to about 100 mg, about 70 mg to about 95 mg, about 70 mg to about 90 mg, about 70 mg to about 85 mg, about 70 mg to about 80 mg, about 70 mg to about 75 mg, about 75 mg to about 100 mg, about 75 mg to about 95 mg, about 75 mg to about 90 mg, about 75 mg to about 85 mg, about 75 mg to about 80 mg, about 80 mg to about 100 mg, about 80 mg to about 95 mg, about 80 mg to about 90 mg, about 80 mg to about 85 mg, about 85 mg to about 100 mg, about 85 mg to about 95 mg, about 85 mg to about 90 mg, about 90 mg to about 100 mg, about 90 mg to about 95 mg, about 95 mg to about 100 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg) BID twice daily, during the period of time. In one embodiment, 30 mg of binimetinib is orally administered twice daily, during the period of time. In one embodiment, 45 mg of binimetinib is orally administered twice daily, during the period of time. In one embodiment, binimetinib is orally administered daily in the amount of about 10 mg to about 100 mg (e.g., any of the subranges or values in this range described herein, e.g., about 30 mg or about 45 mg) BID for three weeks, followed by one week, two weeks, or three weeks without administration of binimetinib in at least one treatment cycle of 28 days, during the period of time. In one embodiment, binimetinib is orally administered daily in the amount of about 30 mg BID for three weeks followed by one week without administration of binimetinib in at least one treatment cycle of 28 days, during the period of time. In one embodiment, binimetinib is orally administered daily in the amount of about 45 mg BID for three weeks followed by one week without administration of binimetinib in at least one treatment cycle of 28 days, during the period of time. In one embodiment, 45 mg of binimetinib is orally administered twice daily until observation of adverse effects, after which 30 mg of binimetinib is administered twice daily, during the period of time. In one embodiment, patients who have been dose reduced to 30 mg twice daily may re-escalate to 45 mg twice daily if the adverse effects that resulted in a dose reduction improve to baseline and remain stable for, e.g., up to 14 days, or up to three weeks, or up to 4 weeks, provided there are no other concomitant toxicities related to binimetinib that would prevent drug re-escalation, during the period of time.

In some embodiments, the PD-1 binding antagonist is nivolumab or a biosimilar thereof. In one embodiment, nivolumab or biosimilar thereof is administered, during the period of time, intravenously at a dose of about 1 mg/mg to about 40 mg/mg (e.g., about 1 mg/kg to about 38 mg/kg, about 1 mg/kg to about 36 mg/kg, about 1 mg/kg to about 34 mg/kg, about 1 mg/kg to about 32 mg/kg, about 1 mg/kg to about 30 mg/kg, about 1 mg/kg to about 28 mg/kg, about 1 mg/kg to about 26 mg/kg, about 1 mg/kg to about 24 mg/kg, about 1 mg/kg to about 22 mg/kg, about 1 mg/kg to about 20 mg/kg, about 1 mg/kg to about 18 mg/kg, about 1 mg/kg to about 16 mg/kg, about 1 mg/kg to about 14 mg/kg, about 1 mg/kg to about 12 mg/kg, about 1 mg/kg to about 10 mg/kg, about 1 mg/kg to about 8 mg/kg, about 1 mg/kg to about 6 mg/kg, about 1 mg/kg to about 4 mg/kg, about 2 mg/kg to about 40 mg/kg, about 2 mg/kg to about 38 mg/kg, about 2 mg/kg to about 36 mg/kg, about 2 mg/kg to about 34 mg/kg, about 2 mg/kg to about 32 mg/kg, about 2 mg/kg to about 30 mg/kg, about 2 mg/kg to about 28 mg/kg, about 2 mg/kg to about 26 mg/kg, about 2 mg/kg to about 24 mg/kg, about 2 mg/kg to about 22 mg/kg, about 2 mg/kg to about 20 mg/kg, about 2 mg/kg to about 18 mg/kg, about 2 mg/kg to about 16 mg/kg, about 2 mg/kg to about 14 mg/kg, about 2 mg/kg to about 12 mg/kg, about 2 mg/kg to about 10 mg/kg, about 2 mg/kg to about 8 mg/kg, about 2 mg/kg to about 6 mg/kg, about 2 mg/kg to about 4 mg/kg, about 4 mg/kg to about 40 mg/kg, about 4 mg/kg to about 38 mg/kg, about 4 mg/kg to about 36 mg/kg, about 4 mg/kg to about 34 mg/kg, about 4 mg/kg to about 32 mg/kg, about 4 mg/kg to about 30 mg/kg, about 4 mg/kg to about 28 mg/kg, about 4 mg/kg to about 26 mg/kg, about 4 mg/kg to about 24 mg/kg, about 4 mg/kg to about 22 mg/kg, about 4 mg/kg to about 20 mg/kg, about 4 mg/kg to about 18 mg/kg, about 4 mg/kg to about 16 mg/kg, about 4 mg/kg to about 14 mg/kg, about 4 mg/kg to about 12 mg/kg, about 4 mg/kg to about 10 mg/kg, about 4 mg/kg to about 8 mg/kg, about 4 mg/kg to about 6 mg/kg, about 6 mg/kg to about 40 mg/kg, about 6 mg/kg to about 38 mg/kg, about 6 mg/kg to about 36 mg/kg, about 6 mg/kg to about 34 mg/kg, about 6 mg/kg to about 32 mg/kg, about 6 mg/kg to about 30 mg/kg, about 6 mg/kg to about 28 mg/kg, about 6 mg/kg to about 26 mg/kg, about 6 mg/kg to about 24 mg/kg, about 6 mg/kg to about 22 mg/kg, about 6 mg/kg to about 20 mg/kg, about 6 mg/kg to about 18 mg/kg, about 6 mg/kg to about 16 mg/kg, about 6 mg/kg to about 14 mg/kg, about 6 mg/kg to about 12 mg/kg, about 6 mg/kg to about 10 mg/kg, about 6 mg/kg to about 8 mg/kg, about 8 mg/kg to about 40 mg/kg, about 8 mg/kg to about 38 mg/kg, about 8 mg/kg to about 36 mg/kg, about 8 mg/kg to about 34 mg/kg, about 8 mg/kg to about 32 mg/kg, about 8 mg/kg to about 30 mg/kg, about 8 mg/kg to about 28 mg/kg, about 8 mg/kg to about 26 mg/kg, about 8 mg/kg to about 24 mg/kg, about 8 mg/kg to about 22 mg/kg, about 8 mg/kg to about 20 mg/kg, about 8 mg/kg to about 18 mg/kg, about 8 mg/kg to about 16 mg/kg, about 8 mg/kg to about 14 mg/kg, about 8 mg/kg to about 12 mg/kg, about 8 mg/kg to about 10 mg/kg, about 10 mg/kg to about 40 mg/kg, about 10 mg/kg to about 38 mg/kg, about 10 mg/kg to about 36 mg/kg, about 10 mg/kg to about 34 mg/kg, about 10 mg/kg to about 32 mg/kg, about 10 mg/kg to about 30 mg/kg, about 10 mg/kg to about 28 mg/kg, about 10 mg/kg to about 26 mg/kg, about 10 mg/kg to about 24 mg/kg, about 10 mg/kg to about 22 mg/kg, about 10 mg/kg to about 20 mg/kg, about 10 mg/kg to about 18 mg/kg, about 10 mg/kg to about 16 mg/kg, about 10 mg/kg to about 14 mg/kg, about 10 mg/kg to about 12 mg/kg, about 12 mg/kg to about 40 mg/kg, about 12 mg/kg to about 38 mg/kg, about 12 mg/kg to about 36 mg/kg, about 12 mg/kg to about 34 mg/kg, about 12 mg/kg to about 32 mg/kg, about 12 mg/kg to about 30 mg/kg, about 12 mg/kg to about 28 mg/kg, about 12 mg/kg to about 26 mg/kg, about 12 mg/kg to about 24 mg/kg, about 12 mg/kg to about 22 mg/kg, about 12 mg/kg to about 20 mg/kg, about 12 mg/kg to about 18 mg/kg, about 12 mg/kg to about 16 mg/kg, about 12 mg/kg to about 14 mg/kg, about 14 mg/kg to about 40 mg/kg, about 14 mg/kg to about 38 mg/kg, about 14 mg/kg to about 36 mg/kg, about 14 mg/kg to about 34 mg/kg, about 14 mg/kg to about 32 mg/kg, about 14 mg/kg to about 30 mg/kg, about 14 mg/kg to about 28 mg/kg, about 14 mg/kg to about 26 mg/kg, about 14 mg/kg to about 24 mg/kg, about 14 mg/kg to about 22 mg/kg, about 14 mg/kg to about 20 mg/kg, about 14 mg/kg to about 18 mg/kg, about 14 mg/kg to about 16 mg/kg, about 16 mg/kg to about 40 mg/kg, about 16 mg/kg to about 38 mg/kg, about 16 mg/kg to about 36 mg/kg, about 16 mg/kg to about 34 mg/kg, about 16 mg/kg to about 32 mg/kg, about 16 mg/kg to about 30 mg/kg, about 16 mg/kg to about 28 mg/kg, about 16 mg/kg to about 26 mg/kg, about 16 mg/kg to about 24 mg/kg, about 16 mg/kg to about 22 mg/kg, about 16 mg/kg to about 20 mg/kg, about 16 mg/kg to about 18 mg/kg, about 18 mg/kg to about 40 mg/kg, about 18 mg/kg to about 38 mg/kg, about 18 mg/kg to about 36 mg/kg, about 18 mg/kg to about 34 mg/kg, about 18 mg/kg to about 32 mg/kg, about 18 mg/kg to about 30 mg/kg, about 18 mg/kg to about 28 mg/kg, about 18 mg/kg to about 26 mg/kg, about 18 mg/kg to about 24 mg/kg, about 18 mg/kg to about 22 mg/kg, about 18 mg/kg to about 20 mg/kg, about 20 mg/kg to about 40 mg/kg, about 20 mg/kg to about 38 mg/kg, about 20 mg/kg to about 36 mg/kg, about 20 mg/kg to about 34 mg/kg, about 20 mg/kg to about 32 mg/kg, about 20 mg/kg to about 30 mg/kg, about 20 mg/kg to about 28 mg/kg, about 20 mg/kg to about 26 mg/kg, about 20 mg/kg to about 24 mg/kg, about 20 mg/kg to about 22 mg/kg, about 22 mg/kg to about 40 mg/kg, about 22 mg/kg to about 38 mg/kg, about 22 mg/kg to about 36 mg/kg, about 22 mg/kg to about 34 mg/kg, about 22 mg/kg to about 32 mg/kg, about 22 mg/kg to about 30 mg/kg, about 22 mg/kg to about 28 mg/kg, about 22 mg/kg to about 26 mg/kg, about 22 mg/kg to about 24 mg/kg, about 24 mg/kg to about 40 mg/kg, about 24 mg/kg to about 38 mg/kg, about 24 mg/kg to about 36 mg/kg, about 24 mg/kg to about 34 mg/kg, about 24 mg/kg to about 32 mg/kg, about 24 mg/kg to about 30 mg/kg, about 24 mg/kg to about 28 mg/kg, about 24 mg/kg to about 26 mg/kg, about 26 mg/kg to about 40 mg/kg, about 26 mg/kg to about 38 mg/kg, about 26 mg/kg to about 36 mg/kg, about 26 mg/kg to about 34 mg/kg, about 26 mg/kg to about 32 mg/kg, about 26 mg/kg to about 30 mg/kg, about 26 mg/kg to about 28 mg/kg, about 28 mg/kg to about 40 mg/kg, about 28 mg/kg to about 38 mg/kg, about 28 mg/kg to about 36 mg/kg, about 28 mg/kg to about 34 mg/kg, about 28 mg/kg to about 32 mg/kg, about 28 mg/kg to about 30 mg/kg, about 30 mg/kg to about 40 mg/kg, about 30 mg/kg to about 38 mg/kg, about 30 mg/kg to about 36 mg/kg, about 30 mg/kg to about 34 mg/kg, about 30 mg/kg to about 32 mg/kg, about 32 mg/kg to about 40 mg/kg, about 32 mg/kg to about 38 mg/kg, about 32 mg/kg to about 36 mg/kg, about 32 mg/kg to about 34 mg/kg, about 34 mg/kg to about 40 mg/kg, about 34 mg/kg to about 38 mg/kg, about 34 mg/kg to about 36 mg/kg, about 36 mg/kg to about 40 mg/kg, about 36 mg/kg to about 38 mg/kg, about 38 mg/kg to about 40 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg at intervals of about 7 days (±2 days), about 14 days (±2 days), or about 21 days (±2 days), or about 30 days (±2 days) during the period of time. In one embodiment, nivolumab or a biosimilar thereof is administered intravenously at a dose of about 3 mg/kg during the period of time. In one embodiment, nivolumab or a biosimilar thereof is administered intravenously as a flat dose of about 20 mg to about 500 mg (e.g., about 20 mg to about 480 mg, about 20 mg to about 460 mg, about 20 mg to about 440 mg, about 20 mg to about 420 mg, about 20 mg to about 400 mg, about 20 mg to about 380 mg, about 20 mg to about 360 mg, about 20 mg to about 340 mg, about 20 mg to about 320 mg, about 20 mg to about 300 mg, about 20 mg to about 280 mg, about 20 mg to about 260 mg, about 20 mg to about 240 mg, about 20 mg to about 220 mg, about 20 mg to about 200 mg, about 20 mg to about 180 mg, about 20 mg to about 160 mg, about 20 mg to about 140 mg, about 20 mg to about 120 mg, about 20 mg to about 100 mg, about 20 mg to about 80 mg, about 20 mg to about 60 mg, about 20 mg to about 40 mg, about 40 mg to about 500 mg, about 40 mg to about 480 mg, about 40 mg to about 460 mg, about 40 mg to about 440 mg, about 40 mg to about 420 mg, about 40 mg to about 400 mg, about 40 mg to about 380 mg, about 40 mg to about 360 mg, about 40 mg to about 340 mg, about 40 mg to about 320 mg, about 40 mg to about 300 mg, about 40 mg to about 280 mg, about 40 mg to about 260 mg, about 40 mg to about 240 mg, about 40 mg to about 220 mg, about 40 mg to about 200 mg, about 40 mg to about 180 mg, about 40 mg to about 160 mg, about 40 mg to about 140 mg, about 40 mg to about 120 mg, about 40 mg to about 100 mg, about 40 mg to about 80 mg, about 40 mg to about 60 mg, about 60 mg to about 500 mg, about 60 mg to about 480 mg, about 60 mg to about 460 mg, about 60 mg to about 440 mg, about 60 mg to about 420 mg, about 60 mg to about 400 mg, about 60 mg to about 380 mg, about 60 mg to about 360 mg, about 60 mg to about 340 mg, about 60 mg to about 320 mg, about 60 mg to about 300 mg, about 60 mg to about 280 mg, about 60 mg to about 260 mg, about 60 mg to about 240 mg, about 60 mg to about 220 mg, about 60 mg to about 200 mg, about 60 mg to about 180 mg, about 60 mg to about 160 mg, about 60 mg to about 140 mg, about 60 mg to about 120 mg, about 60 mg to about 100 mg, about 60 mg to about 80 mg, about 80 mg to about 500 mg, about 80 mg to about 480 mg, about 80 mg to about 460 mg, about 80 mg to about 440 mg, about 80 mg to about 420 mg, about 80 mg to about 400 mg, about 80 mg to about 380 mg, about 80 mg to about 360 mg, about 80 mg to about 340 mg, about 80 mg to about 320 mg, about 80 mg to about 300 mg, about 80 mg to about 280 mg, about 80 mg to about 260 mg, about 80 mg to about 240 mg, about 80 mg to about 220 mg, about 80 mg to about 200 mg, about 80 mg to about 180 mg, about 80 mg to about 160 mg, about 80 mg to about 140 mg, about 80 mg to about 120 mg, about 80 mg to about 100 mg, about 100 mg to about 500 mg, about 100 mg to about 480 mg, about 100 mg to about 460 mg, about 100 mg to about 440 mg, about 100 mg to about 420 mg, about 100 mg to about 400 mg, about 100 mg to about 380 mg, about 100 mg to about 360 mg, about 100 mg to about 340 mg, about 100 mg to about 320 mg, about 100 mg to about 300 mg, about 100 mg to about 280 mg, about 100 mg to about 260 mg, about 100 mg to about 240 mg, about 100 mg to about 220 mg, about 100 mg to about 200 mg, about 100 mg to about 180 mg, about 100 mg to about 160 mg, about 100 mg to about 140 mg, about 100 mg to about 120 mg, about 120 mg to about 500 mg, about 120 mg to about 480 mg, about 120 mg to about 460 mg, about 120 mg to about 440 mg, about 120 mg to about 420 mg, about 120 mg to about 400 mg, about 120 mg to about 380 mg, about 120 mg to about 360 mg, about 120 mg to about 340 mg, about 120 mg to about 320 mg, about 120 mg to about 300 mg, about 120 mg to about 280 mg, about 120 mg to about 260 mg, about 120 mg to about 240 mg, about 120 mg to about 220 mg, about 120 mg to about 200 mg, about 120 mg to about 180 mg, about 120 mg to about 160 mg, about 120 mg to about 140 mg, about 140 mg to about 500 mg, about 140 mg to about 480 mg, about 140 mg to about 460 mg, about 140 mg to about 440 mg, about 140 mg to about 420 mg, about 140 mg to about 400 mg, about 140 mg to about 380 mg, about 140 mg to about 360 mg, about 140 mg to about 340 mg, about 140 mg to about 320 mg, about 140 mg to about 300 mg, about 140 mg to about 280 mg, about 140 mg to about 260 mg, about 140 mg to about 240 mg, about 140 mg to about 220 mg, about 140 mg to about 200 mg, about 140 mg to about 180 mg, about 140 mg to about 160 mg, about 160 mg to about 500 mg, about 160 mg to about 480 mg, about 160 mg to about 460 mg, about 160 mg to about 440 mg, about 160 mg to about 420 mg, about 160 mg to about 400 mg, about 160 mg to about 380 mg, about 160 mg to about 360 mg, about 160 mg to about 340 mg, about 160 mg to about 320 mg, about 160 mg to about 300 mg, about 160 mg to about 280 mg, about 160 mg to about 260 mg, about 160 mg to about 240 mg, about 160 mg to about 220 mg, about 160 mg to about 200 mg, about 160 mg to about 180 mg, about 180 mg to about 500 mg, about 180 mg to about 480 mg, about 180 mg to about 460 mg, about 180 mg to about 440 mg, about 180 mg to about 420 mg, about 180 mg to about 400 mg, about 180 mg to about 380 mg, about 180 mg to about 360 mg, about 180 mg to about 340 mg, about 180 mg to about 320 mg, about 180 mg to about 300 mg, about 180 mg to about 280 mg, about 180 mg to about 260 mg, about 180 mg to about 240 mg, about 180 mg to about 220 mg, about 180 mg to about 200 mg, about 200 mg to about 500 mg, about 200 mg to about 480 mg, about 200 mg to about 460 mg, about 200 mg to about 440 mg, about 200 mg to about 420 mg, about 200 mg to about 400 mg, about 200 mg to about 380 mg, about 200 mg to about 360 mg, about 200 mg to about 340 mg, about 200 mg to about 320 mg, about 200 mg to about 300 mg, about 200 mg to about 280 mg, about 200 mg to about 260 mg, about 200 mg to about 240 mg, about 200 mg to about 220 mg, about 220 mg to about 500 mg, about 220 mg to about 480 mg, about 220 mg to about 460 mg, about 220 mg to about 440 mg, about 220 mg to about 420 mg, about 220 mg to about 400 mg, about 220 mg to about 380 mg, about 220 mg to about 360 mg, about 220 mg to about 340 mg, about 220 mg to about 320 mg, about 220 mg to about 300 mg, about 220 mg to about 280 mg, about 220 mg to about 260 mg, about 220 mg to about 240 mg, about 240 mg to about 500 mg, about 240 mg to about 480 mg, about 240 mg to about 460 mg, about 240 mg to about 440 mg, about 240 mg to about 420 mg, about 240 mg to about 400 mg, about 240 mg to about 380 mg, about 240 mg to about 360 mg, about 240 mg to about 340 mg, about 240 mg to about 320 mg, about 240 mg to about 300 mg, about 240 mg to about 280 mg, about 240 mg to about 260 mg, about 260 mg to about 500 mg, about 260 mg to about 480 mg, about 260 mg to about 460 mg, about 260 mg to about 440 mg, about 260 mg to about 420 mg, about 260 mg to about 400 mg, about 260 mg to about 380 mg, about 260 mg to about 360 mg, about 260 mg to about 340 mg, about 260 mg to about 320 mg, about 260 mg to about 300 mg, about 260 mg to about 280 mg, about 280 mg to about 500 mg, about 280 mg to about 480 mg, about 280 mg to about 460 mg, about 280 mg to about 440 mg, about 280 mg to about 420 mg, about 280 mg to about 400 mg, about 280 mg to about 380 mg, about 280 mg to about 360 mg, about 280 mg to about 340 mg, about 280 mg to about 320 mg, about 280 mg to about 300 mg, about 300 mg to about 500 mg, about 300 mg to about 480 mg, about 300 mg to about 460 mg, about 300 mg to about 440 mg, about 300 mg to about 420 mg, about 300 mg to about 400 mg, about 300 mg to about 380 mg, about 300 mg to about 360 mg, about 300 mg to about 340 mg, about 300 mg to about 320 mg, about 320 mg to about 500 mg, about 320 mg to about 480 mg, about 320 mg to about 460 mg, about 320 mg to about 440 mg, about 320 mg to about 420 mg, about 320 mg to about 400 mg, about 320 mg to about 380 mg, about 320 mg to about 360 mg, about 320 mg to about 340 mg, about 340 mg to about 500 mg, about 340 mg to about 480 mg, about 340 mg to about 460 mg, about 340 mg to about 440 mg, about 340 mg to about 420 mg, about 340 mg to about 400 mg, about 340 mg to about 380 mg, about 340 mg to about 360 mg, about 360 mg to about 500 mg, about 360 mg to about 480 mg, about 360 mg to about 460 mg, about 360 mg to about 440 mg, about 360 mg to about 420 mg, about 360 mg to about 400 mg, about 360 mg to about 380 mg, about 380 mg to about 500 mg, about 380 mg to about 480 mg, about 380 mg to about 460 mg, about 380 mg to about 440 mg, about 380 mg to about 420 mg, about 380 mg to about 400 mg, about 400 mg to about 500 mg, about 400 mg to about 480 mg, about 400 mg to about 460 mg, about 400 mg to about 440 mg, about 400 mg to about 420 mg, about 420 mg to about 500 mg, about 420 mg to about 480 mg, about 420 mg to about 460 mg, about 420 mg to about 440 mg, about 440 mg to about 500 mg, about 440 mg to about 480 mg, about 440 mg to about 460 mg, about 460 mg to about 500 mg, about 460 mg to about 480 mg, about 480 mg to about 500 mg, about 80 mg, about 150 mg, about 160 mg, about 200 mg, about 240 mg, about 250 mg, or about 300 mg), at intervals of about 7 days (±2 days), about 14 days (±2 days), or about 21 days (±2 days), or about 30 days (±2 days) during the period of time. In one embodiment, nivolumab or a biosimilar thereof is administered intravenously as a flat dose of about 240 mg, during the period of time. In one embodiment, nivolumab or a biosimilar thereof is administered intravenously over 60 minutes every two weeks, during the period of time.

In some embodiments, the PD-1 binding antagonist is pembrolizumab or a biosimilar thereof. In one embodiment, pembrolizumab or a biosimilar thereof is administered intravenously at a dose of about 1 mg/mg to about 40 mg/mg (e.g., or any of the subranges of this range described herein, e.g., about 1, 2, 3, 4, 5, 6, 7, 8, 9, 10, 11, 12, 13, 14, 15, 16, 17, 18, 19 or 20 mg/kg) at intervals of about 7 days (±2 days), about 14 days (±2 days), about 21 days (±2 days), or about 30 days (±2 days) during the period of time. In one embodiment, pembrolizumab or a biosimilar thereof is administered intravenously at a dose of about 2 mg/kg, during the period of time. In one embodiment, pembrolizumab or a biosimilar thereof is administered intravenously as a flat dose of about 20 mg to about 500 mg (e.g., or any of the subranges of this range described herein, e.g., about 80, 150, 160, 200, 240, 250, or 300 mg) at intervals of about 7 days (±2 days), about 14 days (±2 days), about 21 days (±2 days), or about 30 days (±2 days) during the period of time. In one embodiment, pembrolizumab or a biosimilar thereof is administered intravenously as a flat dose of about 200 mg, during the period of time. In one embodiment, pembrolizumab or a biosimilar thereof is administered intravenously every three weeks, during the period of time.

In one embodiment, the invention provides a method for treating cancer comprising or consisting essentially of administering to a patient in need thereof, during a period of time, a combination therapy consisting essentially of or consisting of therapeutically effective amounts, independently or in combination, of a MEK inhibitor and a PD-1 binding antagonist, wherein the MEK inhibitor is binimetinib or a pharmaceutically acceptable salt thereof. In one embodiment, the MEK inhibitor is binimetinib as the free base. In one embodiment, the MEK inhibitor is crystallized binimetinib. In one embodiment, binimetinib is orally administered daily in the amount of (i) about 10 mg to about 100 mg (e.g., any of the subranges or values in this range described herein) about 30 mg or about 45 mg) twice a day (BID), during the period of time, or (ii) orally administered daily in the amount of about 10 mg to about 100 mg (e.g., any of the subranges or values in this range described herein, e.g., about 30 mg or about 45 mg) BID for three weeks followed by one week without administration of binimetinib in at least one treatment cycle of 28 days, during the period of time. In one embodiment, the PD-1 binding antagonist is nivolumab or a biosimilar thereof. In one embodiment, the PD-1 binding antagonist is pembrolizumab or a biosimilar thereof. In one embodiment, the amounts of the MEK inhibitor and the PD-1 binding antagonist together achieve a synergistic effect in the treatment of cancer (e.g., during the period of time). In one embodiment, the patient was previously treated with one or more therapeutic agents, e.g., at least one treatment with another anticancer treatment, before the period of time.

In one embodiment, a method for treating cancer comprises or consists essentially of administering to a patient in need thereof, during the period of time, a combination therapy consisting essentially of or consisting of therapeutically effective amounts, independently or in combination, of (a) a MEK inhibitor, which is binimetinib or a pharmaceutically acceptable salt thereof, and (b) a PD-1 binding antagonist which is nivolumab or a biosimilar thereof, wherein nivolumab or a biosimilar thereof is administered intravenously every two weeks during the period of time. In one embodiment, nivolumab or a biosimilar thereof is administered intravenously at a dose of about 3 mg/kg, during the period of time. In one embodiment, nivolumab or a biosimilar thereof is administered intravenously as a flat dose of about 240 mg, during the period of time. In one embodiment, the amounts of binimetinib and nivolumab or a biosimilar thereof together achieve a synergistic effect in the treatment of cancer (e.g., during the period of time). In one embodiment, the subject was previously treated with one or more therapeutic agents, e.g., at least one treatment with another anticancer treatment, before the period of time.

In one embodiment, a method for treating cancer comprises or consists essentially of administering to a patient in need thereof, during a period of time, a combination therapy consisting essentially of or consisting of therapeutically effective amounts, independently or in combination, of (a) a MEK inhibitor, which is binimetinib or a pharmaceutically acceptable salt thereof, and (b) a PD-1 binding antagonist which is pembrolizumab or a biosimilar thereof, wherein pembrolizumab or a biosimilar thereof is administered intravenously every three weeks during the period of time. In one embodiment, pembrolizumab or a biosimilar thereof is administered intravenously at a dose of about 2 mg/kg, during the period of time. In one embodiment, pembrolizumab or a biosimilar thereof is administered intravenously as a flat dose of about 200 mg, during the period of time. In one embodiment, the amounts of binimetinib and nivolumab or a biosimilar thereof together achieve a synergistic effect in the treatment of cancer (e.g., during the period of time). In one embodiment, the subject was previously treated with one or more therapeutic agents, e.g., at least one treatment with another anticancer treatment, before the period of time.

In one embodiment, a method for treating cancer comprises or consists essentially of administering to a patient in need thereof, during a period of time, a combination therapy consisting essentially of or consisting of therapeutically effective amounts, independently or in combination, of (a) a MEK inhibitor, which is binimetinib or a pharmaceutically acceptable salt thereof, wherein binimetinib is orally administered daily in the amount of (i) about 30 mg BID or about 45 mg twice a day (BID), during the period of time, or (ii) orally administered daily in the amount of about 30 mg BID or about 45 mg BID for three weeks followed by one week without administration of binimetinib in at least one treatment cycle of 28 days, during the period of time, and (b) a PD-1 binding antagonist which is nivolumab or a biosimilar thereof, wherein nivolumab or a biosimilar thereof is administered intravenously every two weeks at a dose of about 3 mg/kg or as a flat dose of about 240 mg, during the period of time. In one embodiment, the amounts of binimetinib and nivolumab or a biosimilar thereof together achieve a synergistic effect in the treatment of cancer (e.g., during the period of time). In one embodiment, the subject was previously treated with one or more therapeutic agents, e.g., at least one prior line of treatment, e.g., at least one treatment with another anticancer treatment, before the period of time.

In one embodiment, a method for treating cancer comprises or consists essentially of administering to a patient in need thereof, during a period of time, a combination therapy consisting essentially of or consisting of therapeutically effective amounts, independently or in combination, of (a) a MEK inhibitor, which is binimetinib or a pharmaceutically acceptable salt thereof, wherein binimetinib is orally administered daily in the amount of (i) about 10 mg to about 100 mg (e.g., any subranges or values in this range described herein, e.g., about 30 mg or about 45 mg) twice a day (BID), during the period of time, or (ii) orally administered daily in the amount of about 10 mg to about 100 mg (e.g., any of the subranges or values of this range described herein, e.g., about 30 mg or about 45 mg) BID for three weeks followed by one week without administration of binimetinib in at least one treatment cycle of 28 days, during the period of time, and (b) a PD-1 binding antagonist which is pembrolizumab or a biosimilar thereof, wherein pembrolizumab or a biosimilar thereof is administered intravenously every three weeks, during the period of time. In one embodiment, pembrolizumab or a biosimilar thereof is administered intravenously at a dose of about 2 mg/kg, during the period of time. In one embodiment, pembrolizumab or a biosimilar thereof is administered intravenously as a flat dose of about 200 mg, during the period of time. In one embodiment, the amounts of binimetinib and pembrolizumab or a biosimilar thereof together achieve a synergistic effect in the treatment of cancer (e.g., during the period of time). In one embodiment, the subject was previously treated with one or more therapeutic agents, e.g., at least one prior line of treatment, e.g., at least one treatment with another anticancer treatment, before the period of time.

In an embodiment, the invention is related to a method for treating cancer comprising or consists essentially of administering to a patient in need thereof, during a period of time, a combination therapy consisting essentially of or consisting of an amount of a MEK inhibitor which is binimetinib or a pharmaceutically acceptable salt thereof and an amount of a PD-1 binding antagonist that is effective in treating cancer. In another embodiment, the invention is related to a combination therapy method that consists essentially of administering to a patient in need thereof, over a period of time, a MEK inhibitor which is binimetinib or a pharmaceutically acceptable salt thereof, and a PD-1 binding antagonist. In another embodiment, the invention is related to a method for treating cancer comprising or consisting essentially of administering to a patient in need thereof, over a period of time, a combination therapy consisting essentially of or consisting of an amount of a MEK inhibitor which is binimetinib or a pharmaceutically acceptable salt thereof, and an amount of a PD-1 binding antagonist, wherein the amounts together achieve synergistic effects in the treatment of cancer (e.g., during the period of time). In another embodiment, the invention is related to a combination therapy method consisting essentially of administering to a patient in need thereof, during a period of time, a MEK inhibitor which is binimetinib or a pharmaceutically acceptable salt thereof, and a PD-1 binding, wherein the amounts provide for a syngergistic effect (e.g., in vivo or in vitro, e.g., in an appropriate model cell line or animal model, e.g., those described in the Examples). In one embodiment, the method or use of the invention is related to a synergistic combination therapy consisting essentially of a MEK inhibitor which is binimetinib or a pharmaceutically acceptable salt thereof, in combination with a PD-1 binding antagonist. In one aspect of all the embodiments of this paragraph, the PD-1 binding antagonist is nivolumab. In one aspect of all the embodiments of this paragraph, the PD-1 binding antagonist is pembrolizumab.

Those skilled in the art will be able to determine, according to known methods, the appropriate amount, dose or dosage of each compound, as used in the combination of the present invention, to administer to a patient, taking into account factors such as age, weight, general health, the compound administered, the route of administration, the nature and advancement of the cancer requiring treatment, and the presence of other medications.

The practice of the method of this invention may be accomplished through various administration or dosing regimens. The compounds of the combination of the present invention can be administered concurrently, sequentially, or intermittently, and in any order.

Repetition of the administration or dosing regimens may be conducted as necessary to achieve the desired effect. A “continuous dosing schedule”, as used herein, is an administration or dosing regimen without dose interruptions, e.g., without days off treatment. Repetition of 21 or 28 day treatment cycles without dose interruptions between the treatment cycles is an example of a continuous dosing schedule. In an embodiment, one or both components of the combination of the present invention can be administered in a continuous dosing schedule.

In one embodiment of any of the dosing regimens of a combination therapy as described herein, the second therapeutically effective dose of the MEK inhibitor is administered about 12 hours after the administration of the first dose of the MEK inhibitor, during the period of time. As used herein, the phrase “about 12 hours after the administration of the first dose of the MEK inhibitor” means that the second dose of the MEK inhibitor is administered 10 to 14 hours after the administration of the first dose of the MEK inhibitor, during the period of time.

In one embodiment, of any of the dosing regimens of a combination therapy as described herein, on days when the PD-1 binding antagonist is administered during the period of time, the PD-1 binding antagonist is administered at least 30 minutes after the administration of a therapeutically effective amount of the first therapeutically effective dose of the MEK inhibitor, wherein the MEK inhibitor is administered twice daily, during the period of time. As used herein, the phrase “at least 30 minutes after” means that the PD-1 binding antagonist is administered during the period of time at least 5 minutes, or at least 10 minutes, or at least 15 minutes, or at least 20 minutes, or at least 25 minutes, or at least 30 minutes, or at least 35 minutes, or at least 40 minutes, or at least 45 minutes, or at least 50 minutes, or at least 55 minutes, or at least 60 minutes, or at least 65 minutes, or at least 70 minutes, or at least 75 minutes, or at least 80 minutes, or at least 85 minutes, or at least 90 minutes after the administration of the first dose of the MEK inhibitor, during the period of time.

In one embodiment of any of the dosing regimens of a combination therapy as described herein, on days when the PD-1 binding antagonist is administered, during the period of time, the PD-1 binding antagonist is administered at least 30 minutes before the administration of a therapeutically effective amount of the first therapeutically effective dose of the MEK inhibitor, during the period of time. As used herein, the phrase “at least 30 minutes after” means that the PD-1 binding antagonist is administered during the period of time at least 5 minutes, or at least 10 minutes, or at least 15 minutes, or at least 20 minutes, or at least 25 minutes, or at least 30 minutes, or at least 35 minutes, or at least 40 minutes, or at least 45 minutes, or at least 50 minutes, or at least 55 minutes, or at least 60 minutes, or at least 65 minutes, or at least 70 minutes, or at least 75 minutes, or at least 80 minutes, or at least 85 minutes, or at least 90 minutes before administration of the first dose of the MEK inhibitor, during the period of time.

In one embodiment, the dose of the MEK inhibitor is escalated during the period of time until the Maximum Tolerated Dosage is reached, and the PD-1 binding antagonist is administered as a fixed dose, during the period of time. Alternatively, the MEK inhibitor may be administered as a fixed dose during the period of time and the dose of the PD-1 binding antagonist may be escalated until the Maximum Tolerated Dosage is reached, during the period of time.

In one embodiment, any combination therapy described herein may further comprise administration of one or more pre-medications prior to the administration of the PD-1 binding antagonist, during the period of time. In one embodiment, the one or more pre-medication(s) is administered during the period of time no sooner than 1 hour after administration of the MEK inhibitor. In one embodiment, the one or more premedication(s) is administered 30-60 minutes prior to the administration of the PD-1 binding antagonist, during the period of time. In one embodiment, the one or more premedication(s) is administered 30 minutes prior administration of the PD-1 binding antagonist, during the period of time. In one embodiment, the one or more pre-medications is selected from one or more of a Hi antagonist (e.g., antihistamines such as diphenhydramine) and acetaminophen.

In one embodiment, the one or more therapeutic agents that are administered to the patient before the period of time is or includes chemotherapy. In one embodiment, the one or more therapeutic agents that are administered to the patient before the period of time is or includes a platinum-based chemotherapy. In one embodiment, the one or more therapeutic agents that are administered to the patient before the period of time is or includes a fluoropyrimidine-containing chemotherapy. In one embodiment, the one or more therapeutic agents that are administered to the patient before the period of time is or includes FOLFIRINOX (a chemotherapy regimen of folinic acid (leucovorin), fluorouracil (5-FU), irinotecan, and oxaliplatin). In one embodiment, the one or more therapeutic agents that are administered to the patient before the period of time is or includes FOLFOXIRI (a chemotherapy regimen of irinotecan and oxaliplatin plus 5-fluorouracil). In one embodiment, the cancer has progressed after treatment with a platinum-based chemotherapy.

An improvement in a cancer or cancer-related disease can be characterized as a complete or partial response. “Complete response” or “CR” refers to an absence of clinically detectable disease with normalization of any previously abnormal radiographic studies, bone marrow, and cerebrospinal fluid (CSF) or abnormal monoclonal protein measurements. “Partial response” refers to at least about a 10%, 20%, 30%, 40%, 50%, 60%, 70%, 80% or 90% decrease in all measurable tumor burden (i.e., the number of malignant cells present in the subject, or the measured bulk of tumor masses or the quantity of abnormal monoclonal protein) in the absence of new lesions.

Treatment may be assessed with one or more clinical endpoints, for example by inhibition of disease progression, inhibition of tumor growth, reduction of primary tumor, relief of tumor-related symptoms, inhibition of tumor secreted factors (including expression levels of checkpoint proteins as identified herein), delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), improved Time to tumor response (TTR), increased duration of response (DR), increased Progression Free Survival (PFS), increased Overall Survival (OS), Objective Response Rate (ORR), among others. OS as used herein means the time from treatment onset until death from any cause. TTP as used herein means the time from treatment onset until tumor progression; TTP does not comprise deaths. As used herein, TTR is defined for patients with confirmed objective response (CR or PR) as the time from the date of randomization or date of first dose of study treatment to the first documentation of objective tumor response. As used herein, DR means the time from documentation of tumor response to disease progression. As used herein, PFS means the time from treatment onset until tumor progression or death. As used herein, ORR means the proportion of patients with tumor size reduction of a predefined amount and for a minimum time period, where response duration usually is measured from the time of initial response until documented tumor progression. In the extreme, complete inhibition, is referred to herein as prevention or chemoprevention.

Thus, provided herein are methods for achieving one or more clinical endpoints associated with treating a cancer with a combination therapy described herein. In one embodiment, a patient described herein can show a positive tumor response, such as inhibition of tumor growth or a reduction in tumor size after treatment with a combination described herein. In certain embodiments, a patient described herein can achieve a Response Evaluation Criteria in Solid Tumors (for example, RECIST 1.1) of complete response, partial response or stable disease after administration of an effective amount a combination therapy described herein. In certain embodiments, a patient described herein can show increased survival without tumor progression. In some embodiments, a patient described herein can show inhibition of disease progression, inhibition of tumor growth, reduction of primary tumor, relief of tumor-related symptoms, inhibition of tumor secreted factors (including tumor secreted hormones, such as those that contribute to carcinoid syndrome), delayed appearance of primary or secondary tumors, slowed development of primary or secondary tumors, decreased occurrence of primary or secondary tumors, slowed or decreased severity of secondary effects of disease, arrested tumor growth and regression of tumors, decreased Time to Tumor Response (TTR), increased Duration of Response (DR), increased Progression Free Survival (PFS), increased Time To Progression (TTP), and/or increased Overall Survival (OS), among others.

In another embodiment, methods are provided for decreasing Time to Tumor Response (TTR), increasing Duration of Response (DR), increasing Progression Free Survival (PFS) of a patient having a cancer described herein, comprising administering an effective amount of a combination therapy as described herein. In one embodiment, a method is provided for decreasing Time to Tumor Response (TTR) of a patient having a cancer described herein, comprising administering an effective amount of a combination therapy as described herein. In one embodiment, is a method for increasing Progression Free Survival (PFS) of a patient a cancer described herein, comprising administering an effective amount of a combination therapy as described herein. In one embodiment, is a method for increasing Progression Free Survival (PFS) of a patient having a cancer described herein, comprising administering an effective amount of a combination therapy as described herein.

In one embodiment, the methods of treating cancer according to the invention also include surgery or radiotherapy. Non-limiting examples of surgery include, e.g., open surgery or minimally invasive surgery. Surgery can include, e.g., removing an entire tumor, debulking of a tumor, or removing a tumor that is causing pain or pressure in the subject. Methods for performing open surgery and minimally invasive surgery on a subject having a cancer are known in the art. Non-limiting examples of radiation therapy include external radiation beam therapy (e.g., external beam therapy using kilovoltage X-rays or megavoltage X-rays) or internal radiation therapy. Internal radiation therapy (also called brachytherapy) can include the use of, e.g., low-dose internal radiation therapy or high-dose internal radiation therapy. Low-dose internal radiation therapy includes, e.g., inserting small radioactive pellets (also called seeds) into or proximal to a cancer tissue in the subject. High-dose internal radiation therapy includes, e.g., inserting a thin tube (e.g., a catheter) or an implant into or proximal to a cancer tissue in the subject, and delivering a high dose of radiation to the thin tube or implant using a radiation machine. Methods for performing radiation therapy on a subject having a cancer are known in the art.

It may be shown by established test models that a combination therapy described herein results in the beneficial effects described herein before. The person skilled in the art is fully enabled to select a relevant test model to prove such beneficial effects. The pharmacological activity of a combination therapy described herein may, for example, be demonstrated in an animal model and/or a clinical study or in a test procedure, for example as described below.

Suitable clinical studies are, for example, open label, dose escalation studies in patients with a proliferative disease. Such studies may demonstrate in particular the synergism of the therapeutic agents of a combination therapy described herein. The beneficial effects on proliferative diseases may be determined directly through the results of these studies. Such studies may, in particular, be suitable for comparing the effects of a monotherapy using the MEK inhibitor and/or the PD-1 binding antagonist versus the effects of a combination therapy comprising the MEK inhibitor and the PD-1 binding antagonist.

The efficacy of the treatment may be determined in such studies, e.g., after 6, 12, 18 or 24 weeks by evaluation of symptom scores, e.g., every 6 weeks.

In some embodiments of any of the methods described herein, the patient is identified as having a tumor or a cancer cell that has increased level of PD-L1 and/or PD-L2 protein, e.g., as compared to a non-cancerous cell. Methods for determining a level of PD-L1 and PD-L2 in a tumor (e.g., a biopsy sample) or cancer cell are known in the art. Such methods include, e.g., immunoblotting, protein array, mass spectrometry, immunofluorescence microscopy, and fluorescence-assisted cell sorting (FACS). Additional methods for determining a level of PD-L1 and PD-L2 in a tumor (e.g., a biopsy sample) or a cancer cell are known in the art. Some embodiments of any of the methods described herein further include identifying a patient as having a tumor or a cancer cell that has an increased level of PD-L1 and/or PD-L2, and selecting the identified patient for treatment using any of the methods described herein. Some embodiments of any of the methods described herein can further include a step of selecting a subject identified as having a tumor or a cancer cell that has an increased level of PD-L1 and/or PD-L2, and the treating the patient using any of the methods described herein.

In some embodiments of any of the methods described herein, the patient is identified as having a tumor or a cancer cell having an upregulated level of MEK, a mutated MEK having increased activity as compared to a wildtype MEK, an upregulated level of a kinase upstream of MEK kinase (e.g., Ras (KRAS, HRAS, and/or NRAS) and/or Raf), or a mutated kinase upstream of MEK (e.g., Ras and/or Raf) having increased activity as compared to the corresponding wildtype kinase upstream of MEK.

In some embodiments, a mutated MEK having increased activity as compared to a wildtype MEK can have, e.g., one or more amino acid substitutions at an amino acid positions selected from the group of 56 (e.g., Q56P) and 72 (e.g., S72G).

In some embodiments, a mutated KRAS having increased activity as compared to a wildtype KRAS can have, e.g., one or more amino acid substitutions at amino acid position 12 (e.g., G12A, G12R, G12S, G12C, G12D or G12V), 13 (e.g., G13D or G13C).

In some embodiments, a mutated HRAS having increased activity as compared to a wildtype HRAS can have, e.g., one or both of amino acid substitutions at amino acid positions 12 (e.g., G12V) and 61 (e.g., Q61 L or Q61R).

In some embodiments, a mutated NRAS having increased activity as compared to a wildtype NRAS can have, e.g., an amino acid substitution at one or more of amino acid positions 12 (e.g., G12D, G12S, or G12V), 13 (e.g., G13R or G13V), and 61 (e.g., Q61H, Q61K, Q61 L, or Q61R).

In some embodiments, a mutated BRAF having increased activity as compared to a wildtype BRAF can have, e.g., an amino acid substitution at amino acid position 600 (e.g., V600E or V600K).

Methods for detecting an increased level of MEK, Ras, and/or Raf, or expression of a mutated MEK, Ras, and/or Raf that has increased activity as compared to the corresponding wildtype kinase in a tumor (e.g., a biopsy sample) or a cancer cell are known in the art and include, e.g., nucleic acid sequencing (e.g., PCR), fluorescence in situ hybridization (FISH) with a labeled DNA probe, immunofluorescence microscopy, immunoblotting, proteomics, mass spectrometry, and fluorescence-assisted cell sorting. Additional methods for detecting an increased level of MEK, Ras, and/or Raf, or expression of a mutated MEK, Ras, and/or Raf that has increased activity as compared to the corresponding wildtype kinase in a tumor (e.g., a biopsy sample) or a cancer cell are known in the art.

Some embodiments of any of the methods described herein further include identifying a patient as having a tumor or a cancer cell that has an increased level of MEK, Ras, and/or Raf, or expresses a mutated MEK, Ras, and/or Raf that has increased activity as compared to the corresponding wildtype kinase, and selecting the identified patient for treatment using any of the methods described herein. Some embodiments of any of the methods described herein can further include a step of selecting a subject identified as having a tumor or a cancer cell that has an increased level of MEK, Ras, and/or Raf, or expresses a mutated MEK, Ras, and/or Raf that has increased activity as compared to the corresponding wildtype kinase, and the treating the patient using any of the methods described herein.

In some embodiments of any of the methods described herein, the patient is identified as having a tumor or a cancer cell that has a decreased level of MHC class I, e.g., as compared to a non-cancerous cell. Methods for determining a level of MHC class I in a tumor (e.g., a biopsy sample) or cancer cell are known in the art. Such methods include, e.g., immunoblotting, protein array, mass spectrometry, immunofluorescence microscopy, and fluorescence-assisted cell sorting (FACS). Additional methods for determining a level of MHC class I in a tumor (e.g., a biopsy sample) or a cancer cell are known in the art. Some embodiments of any of the methods described herein further include identifying a patient as having a tumor or a cancer cell that has a decreased level of MHC class I, and selecting the identified patient for treatment using any of the methods described herein. Some embodiments of any of the methods described herein can further include a step of selecting a subject identified as having a tumor or a cancer cell that has a decreased level of MHC class I, and the treating the patient using any of the methods described herein.

In some embodiments, the cancer is selected from the group consisting of: pancreatic cancer, breast cancer (e.g., triple-negative breast cancer), mantle cell lymphoma, non-small cell lung cancer, melanoma, colon cancer, esophageal cancer, liposarcoma, multiple myeloma, T-cell leukemia, renal cell carcinoma, gastric cancer, glioblastoma, hepatocellular cancer, hepatocellular carcinoma, lung cancer, colorectal cancer, rhabdoid tumor, retinoblastoma proteinpositive cancers, gall bladder cancer, cholangiocarcinoma, astrocytomas, glioblastoma multiforme, Bannayan-Zonana syndrome, Cowden disease, Lhermitte-Duclos disease, Wilm's tumor, Ewing's sarcoma, Rhabdomyosarcoma, ependymoma, medulloblastoma, head and neck, kidney cancer, ovarian cancer, prostate cancer, sarcoma, osteosarcoma, giant cell tumor of bone, thyroid, Chronic myelogenous leukemia, Chronic lymphocytic leukemia, Hairy-cell leukemia, acute lymphoblastic leukemia, acute myelogenous leukemia, AML, Chronic neutrophilic leukemia, plasmacytoma, Immunoblastic large cell leukemia, Mantle cell leukemia, Megakaryoblastic leukemia, acute megakaryocytic leukemia, promyelocytic leukemia, Erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, advanced urothelial bladder cancer, urothelial carcinoma, lung cancer, vulval cancer, cervical cancer, endometrial cancer, renal cancer, mesothelioma, salivary gland cancer, nasopharangeal cancer, buccal cancer, cancer of the mouth, GIST (gastrointestinal stromal tumor), NSCLC, and testicular cancer. In some embodiments, the cancer is a T-cell infiltrating cancer.

In some embodiments of any of the methods described herein, the cancer is breast cancer, ovarian cancer, endometrial cancer, cervical cancer, acute myeloid leukemia, chronic myelocytic leukemia, myelodysplasia, hepatocellular cancer, idiopathic myelofibrosis, myelomonoblastic leukemia, pigmented villonodular synovitis, tenosynovial giant cell tumors, multiple myeloma, lung cancer, prostate cancer, gastric cancer, bladder cancer, Kaposi's sarcoma, or ovarian cancer.

In some embodiments of any of the methods described herein, the cancer is lung cancer, non small cell lung (NSCL) cancer, bronchioloalveolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, lymphoma, or ymphocytic leukemia, including refractory versions of any of the above cancers.

In some embodiments, the cancer is selected from the group consisting of: renal cancer, lung cancer, head and neck cancer, classical Hodgkin lymphoma, colon cancer, pancreatic cancer, breast cancer, prostate cancer, lung cancer, brain cancer, ovarian cancer, cervical cancer, testicular cancer, renal cancer, lymphoma, leukemia, melanoma, non-small cell lung cancer (NSCLC), colon cancer, colon carcinoma, colorectal carcinoma (e.g., microsatellite instability—high/mismatch repair deficient colorectal cancer), skin cancer, metastatic melanoma, breast cancer, liver cancer, hepatoma, stomach cancer, head and neck cancer, bladder cancer, haematological cancer, lymphoma, and Hodgkin's lymphoma, osteosarcoma, neuroblastoma, glioma, glioblastoma multiforme, epitheloid carcinoma, esophageal cancer, and rectal cancer.

The compounds of the method or combination of the present invention may be formulated prior to administration. The formulation will preferably be adapted to the particular mode of administration. These compounds may be formulated with pharmaceutically acceptable carriers as known in the art and administered in a wide variety of dosage forms as known in the art. In making the pharmaceutical compositions of the present invention, the active ingredient will usually be mixed with a pharmaceutically acceptable carrier, or diluted by a carrier or enclosed within a carrier. Such carriers include, but are not limited to, solid diluents or fillers, excipients, sterile aqueous media and various non-toxic organic solvents. Dosage unit forms or pharmaceutical compositions include tablets, capsules, such as gelatin capsules, pills, powders, granules, aqueous and nonaqueous oral solutions and suspensions, lozenges, troches, hard candies, sprays, creams, salves, suppositories, jellies, gels, pastes, lotions, ointments, injectable solutions, elixirs, syrups, and parenteral solutions packaged in containers adapted for subdivision into individual doses.

Parenteral formulations include pharmaceutically acceptable aqueous or nonaqueous solutions, dispersion, suspensions, emulsions, and sterile powders for the preparation thereof. Examples of carriers include water, ethanol, polyols (propylene glycol, polyethylene glycol), vegetable oils, and injectable organic esters such as ethyl oleate. Fluidity can be maintained by the use of a coating such as lecithin, a surfactant, or maintaining appropriate particle size. Exemplary parenteral administration forms include solutions or suspensions of the compounds of the invention in sterile aqueous solutions, for example, aqueous propylene glycol or dextrose solutions. Such dosage forms can be suitably buffered, if desired.

Additionally, lubricating agents such as magnesium stearate, sodium lauryl sulfate and talc are often useful for tableting purposes. Solid compositions of a similar type may also be employed in soft and hard filled gelatin capsules. Preferred materials, therefor, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art.

In one embodiment, the MEK inhibitor which is binimetinib or a pharmaceutically acceptable salt thereof is formulated for oral administration. In one embodiment, the MEK inhibitor is formulated as a tablet or capsule. In one embodiment, the MEK inhibitor is formulated as a tablet. In one embodiment, the tablet is a coated tablet. In one embodiment, the MEK inhibitor is binimetinib as the fee base. In one embodiment, the MEK inhibitor is a pharmaceutically acceptable salt of binimetinib. In one embodiment, the MEK inhibitor is crystallized binimetinib. Methods of preparing oral formulations of binimetinib are described in PCT publication No. WO 2014/063024. In one embodiment, a tablet formulation of binimetinib comprises 15 mg of binimetinib. In one embodiment, a tablet formulation of binimetinib comprises 15 mg of crystallized binimetinib. In one embodiment, a tablet formulation of binimetinib comprises 45 mg of binimetinib. In one embodiment, a tablet formulation of binimetinib comprises 45 mg of crystallized binimetinib.

The invention also relates to a kit comprising the therapeutic agents of the combination of the present invention and written instructions for administration of the therapeutic agents. In one embodiment, the written instructions elaborate and qualify the modes of administration of the therapeutic agents, for example, for simultaneous or sequential administration of the therapeutic agents of the present invention. In one embodiment, the written instructions elaborate and qualify the modes of administration of the therapeutic agents, for example, by specifying the days of administration for each of the therapeutic agents during a 28 day cycle.

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

### EXAMPLES

### Example 1. Effect of Binimetinib on MHC Class I Expression in Melanoma Cell Lines

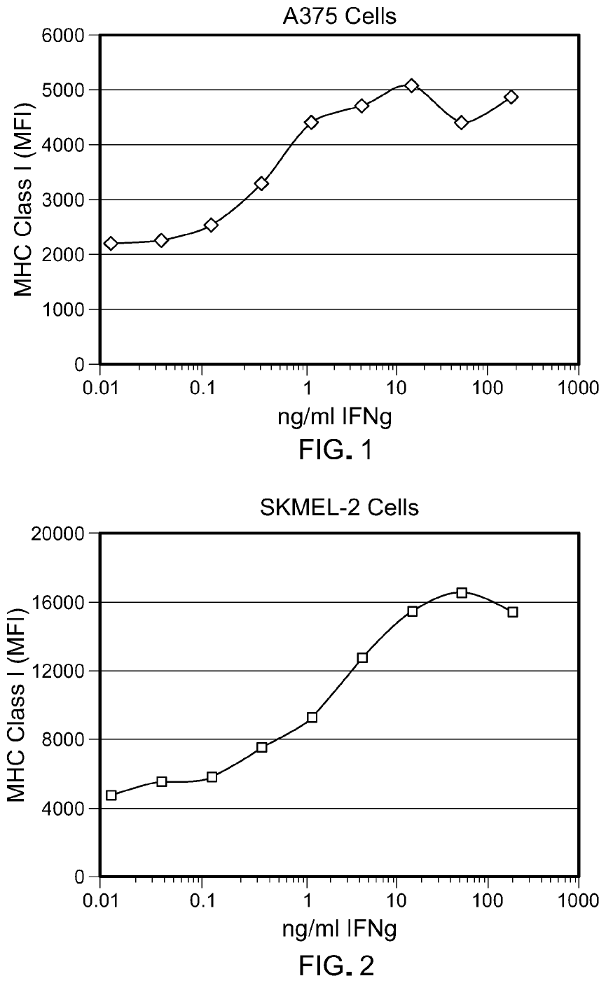

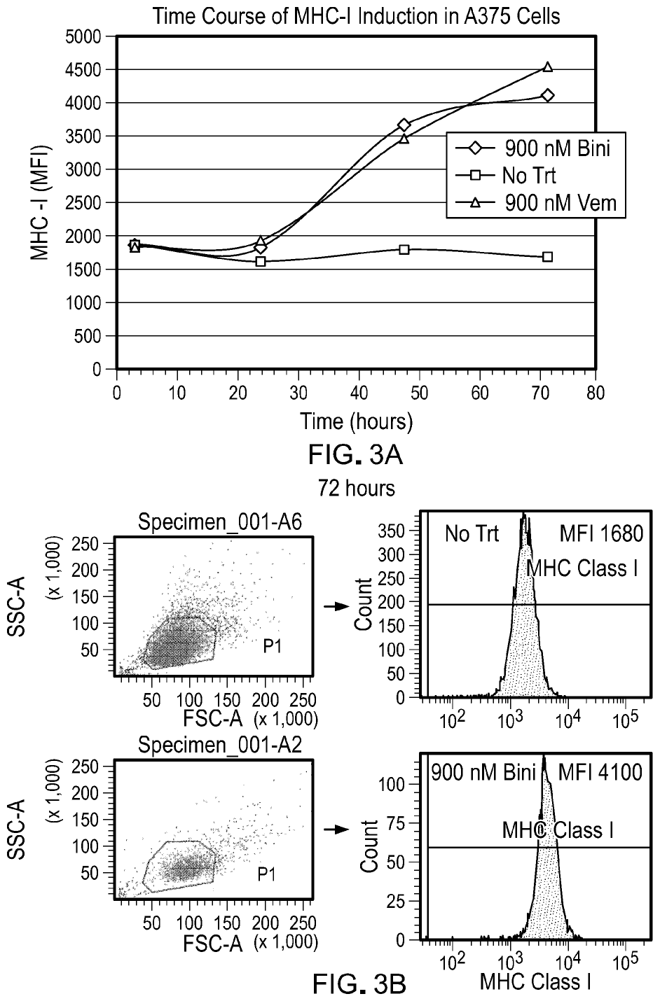

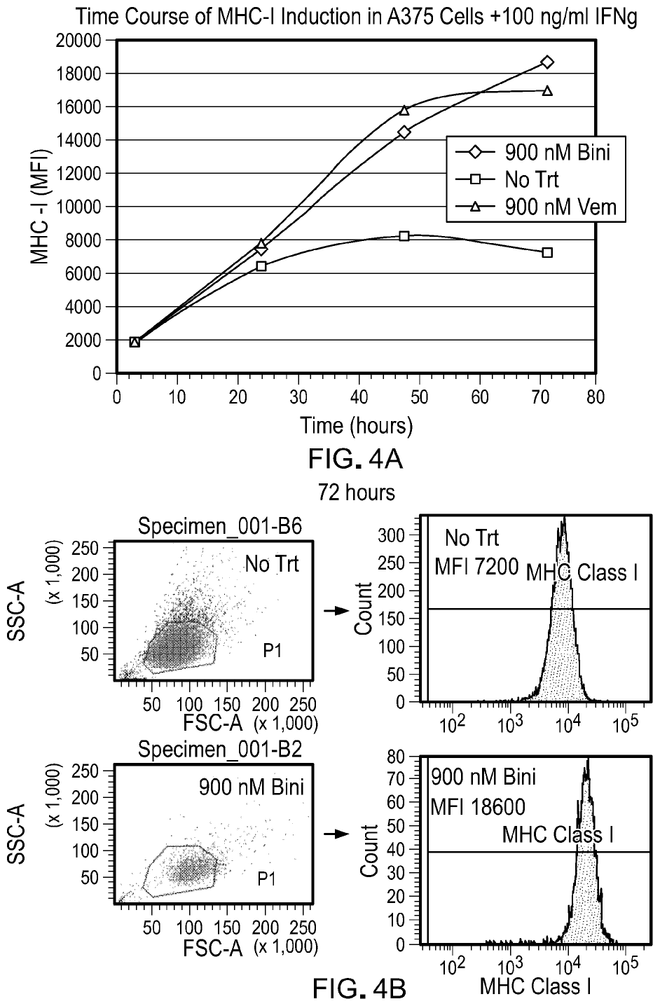

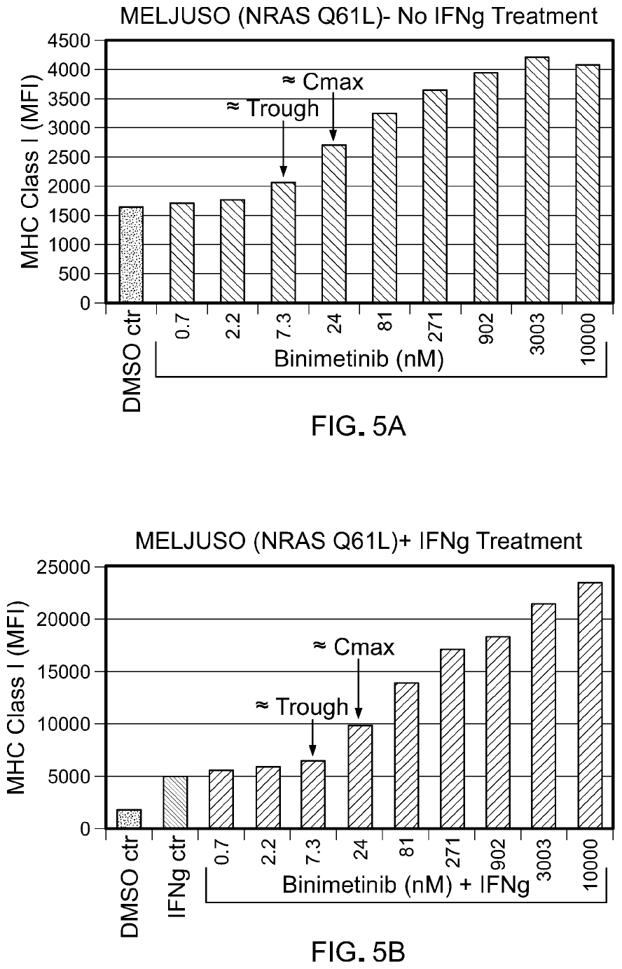

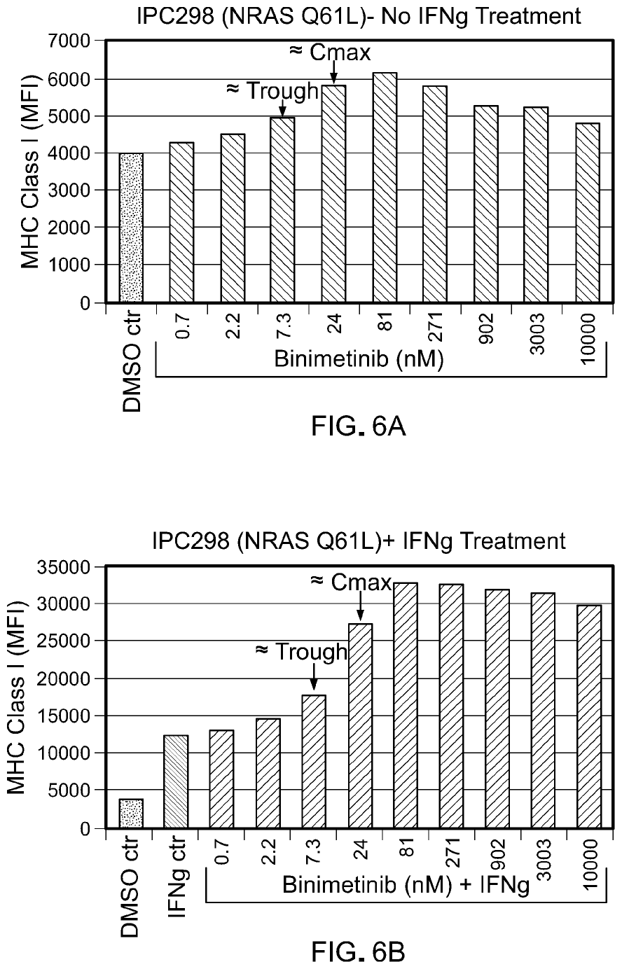

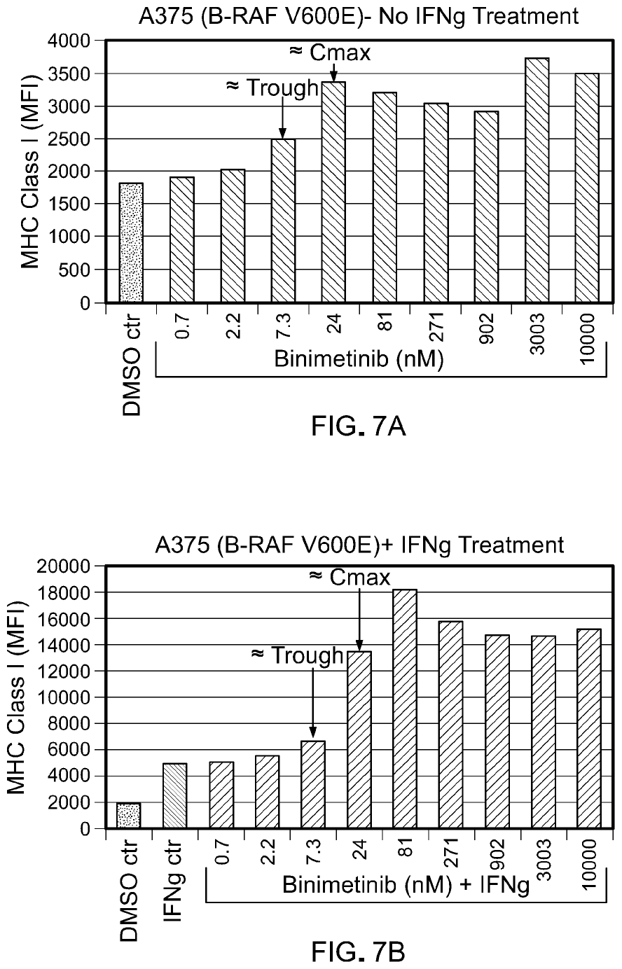

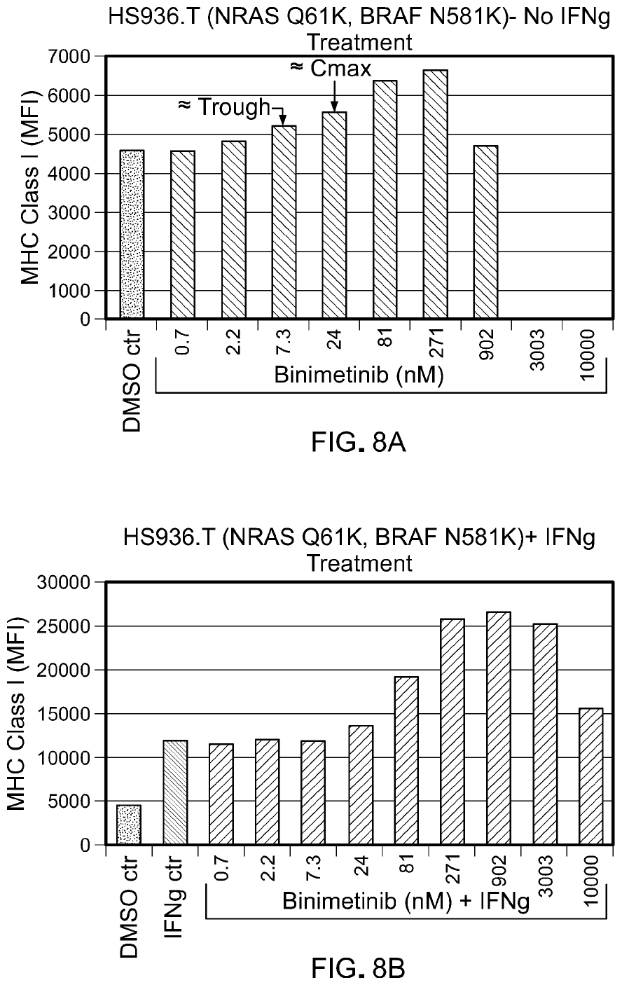

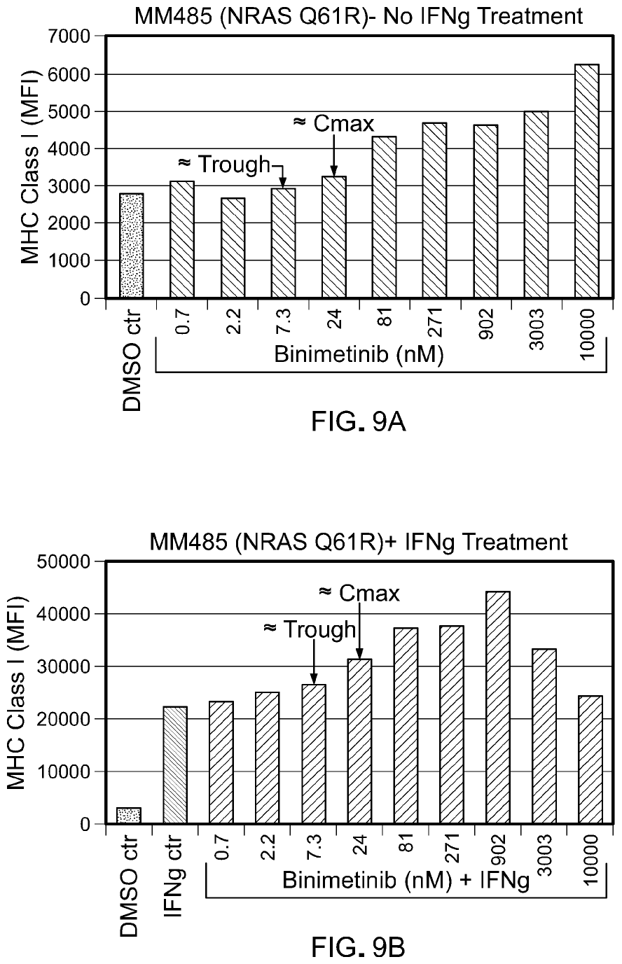

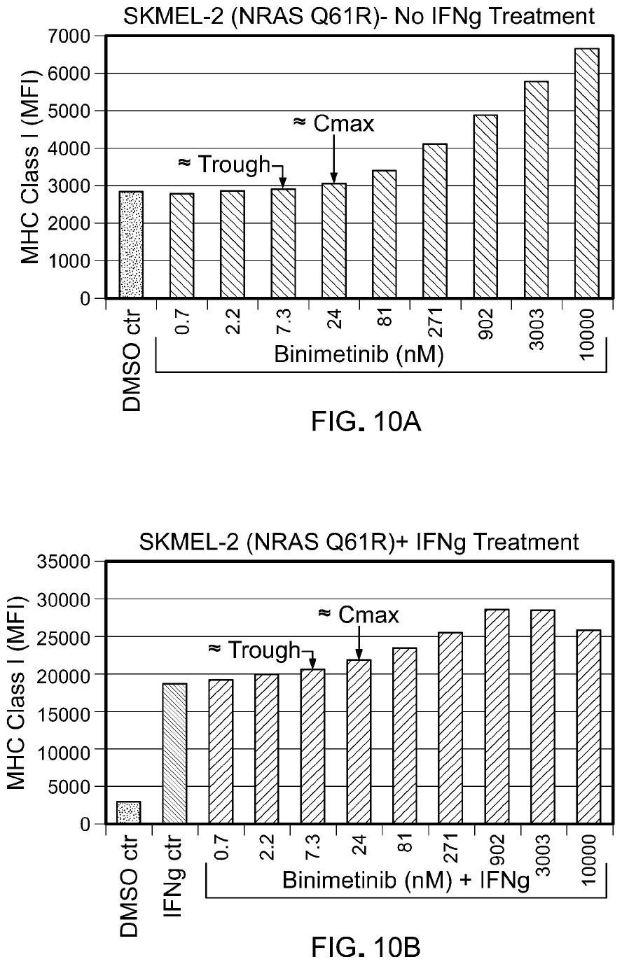

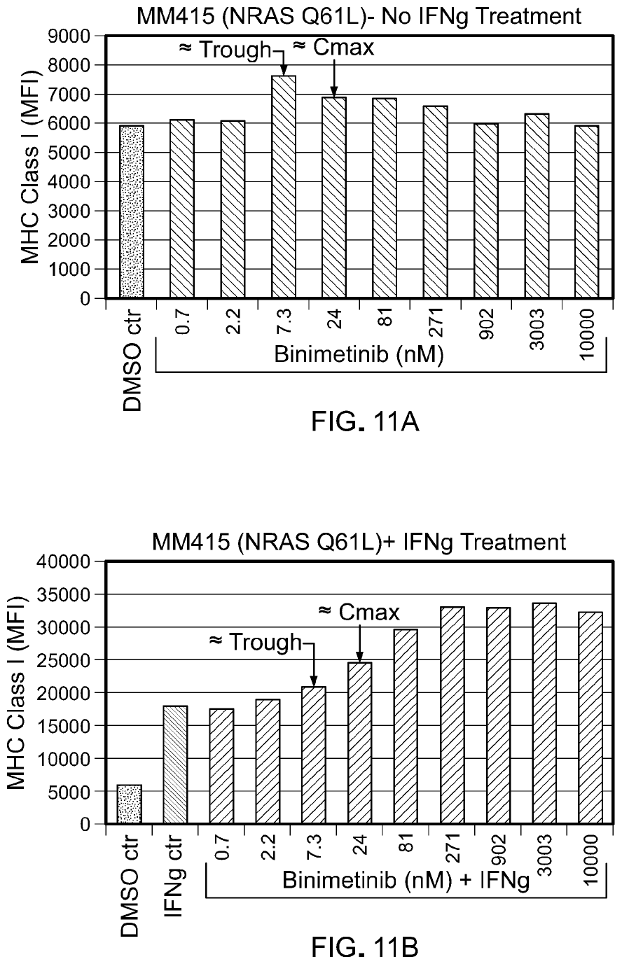

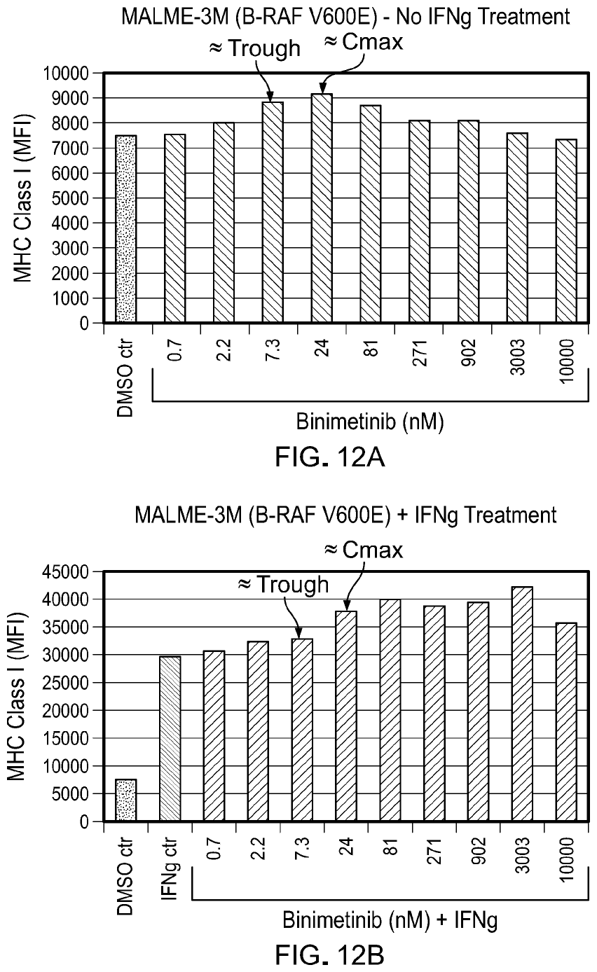

The goal of this study was to measure the effects of binimetinib on cell surface MHC class I expression in various melanoma cell lines. In this study, eight melanoma cell lines were evaluated: MEL-JUSO (NRAS Q61L mutation), IPC-298 (NRAS Q61L mutation), A375 (BRAF V600E, homozygous mutation), HS936.T (NRAS Q61K mutation and BRAF N581K mutation), MM485 (NRAS Q61R mutation), SK-MEL-2 (NRAS Q61R mutation), MM415 (NRAS Q61L mutation), Malme-3M (BRAF V600E, heterozygous mutation). The cells lines were seeded into flat bottom 96-well tissue culture plates at a density of 5000 cells per well and allowed to adhere overnight. The following day cells were treated with vehicle control (0.25% DMSO) or varying dilutions of Binimetinib (0.7 nM-25000 nM) for 1 hour. Cells were then treated with vehicle control or 100 ng/ml IFNgamma (R&D systems) for 72 hours. Cells were washed, trypsinized, and stained with Alexa Fluor® 647 anti-human HLA-A,B,C antibody (W6/32, eBioscience) and analyzed on a BD FACSCanto II flow cytometer. An IFNg titration and time course were performed for assay optimization. The results of this study are shown in FIGS. 1-12. Maximal induction of MHC class I expression was seen ˜100 ng/mL of IFNγ (see, e.g., FIGS. 1 and 2). As shown in FIG. 5A, binimetinib treatment led to a 2.5-fold increase in MHC class I expression in MELJUSO (NRAS Q61L) cells, whereas binimetinib treatment in the presence of 100 ng/mL IFNg led to a 4-5 fold enhancement of IFNγ-induced MHC class I expression. Binimetinib treatment resulted in increased MHC class I cell surface expression in 6 of 8 melanoma cell lines (˜1.5-3 fold increase) and enhanced IFNg-induced MHC class I in all tested cell lines (˜1.5-4 fold increase) (Table 3).

TABLE 3
Maximal fold increase in MHC class I expression

| | Maximal Fold Increase in MHC Class | |
|---|---|---|
| | I Expression | |
| Cell Line | −IFNg | +IFNg |
| MELJUSO (NRAS Q61L) | 3 | 4 |
| IPC298 (NRAS Q61L) | 2 | 3 |
| A375 (BRAF V600E) | 2 | 4 |
| HS936.T (NRAS Q61K, | 1.5 | 2 |
| BRAF N581K) | | |
| MM485 (NRAS Q61R) | 2 | 2 |
| SKMEL-2 (NRAS Q61R) | 2 | 1.5 |
| MM415 (NRAS Q61L) | 0 | 2 |
| Malme-3M (BRAF V600E) | 0 | 1.5 |

### Example 2. Exploratory Study Evaluating Sequence Dependence of Binimetinib and α-PD-1 Combinatorial Efficacy on Growth of KRas Mutant CT26 Murine Colon Syngeneic Tumors in BALB/c Mice

The PD-1/PDL-1 pathway regulates immune expression by multiple mechanisms (induction of T-cell apoptosis, promotion of T-cell exhaustion, inhibition of T-cell proliferation etc.). Signaling through PD-1 prevents the conversion of functional CD8+T effector memory cells into CD8+ central memory cell. This reduces long-term immune memory, which might protect against future metastatic disease. Therefore, inhibition of the PD-1/PD-L1 pathway may enhance long-term immune memory. Nonclinical studies in syngeneic mouse tumor models evaluating re-challenge with tumor cells post anti-PD-1 therapy demonstrated complete inhibition of tumor growth in response to the re-introduction of viable tumor cells (Lu et al., Journal of Translational Medicine 2014, 12: 36-47; Sagiv-Barfia et al., PNAS 2015, 112: E966-972; Shindo et al., Anticancer research 2015, 35: 129-136).

This study was designed to determine if MEK162 (binimetinib) could be administered in combination, either concomitantly or sequentially, with anti-PD1 in an immune-competent model of KRas mutant colorectal carcinoma.

### Experimental Methods

### Compounds

1% CMC/0.5% Tween-80 (CMCT) was prepared by adding 960 mL of distilled water to a 1000 mL Pyrex® glass bottle with a stir bar in the bottom, and was used as the oral vehicle. The water was warmed to 50-60° C. then 5 mL Tween® 80 (polyoxyethylenesorbitan monooleate, Sigma P2287, batch #054K0154) and 15.6 mL benzyl alcohol (Sigma 402834, Batch #00296PK) added. Carboxymethylcellulose (5 g, CMC, low viscosity, Sigma C5678, Batch #033K0008) was added slowly, over one hour. The solution was stirred until the CMC dissolved and the solution was clear. 30 mg/kg MEK162 (Binimetinib, 100% active) was prepared as a white, homogeneous suspension in CMCT. MEK162 was administered in 10 mL/kg and dosed at 30 mg/kg (3.0 mg/mL) by oral gavage. To prepare, dry test compound (90 mg) was weighed and 30 mL CMCT added. Dose suspension was sonicated until a fine suspension was achieved (˜15 min). Final dose suspension was stored at 4° C. during live phase and sonicated to resuspend prior to dose administration. Dose suspension was prepared every 4-5 days or as needed. 100 μg αPD-1 RMP1-14, Rat IgG2a isotype (BioXCell cat.no. BP0146, lot #5792×2/1015)−7.12 mg/mL stock concentration. RMP1-14 was prepared by dilution of 0.843 stock solution with 5.157 mL sterile saline for injection. The antibody was administered at 100 μg/animal (100 μL of a 1 mg/mL dose solution) by intraperitoneal injection.

### Experimental Animals

Male BALB/c mice from Charles River (Wilmington, Mass.) were obtained at 6-8 weeks of age and housed in groups of 5. Following a two week acclimation period, a suspension of 1×10⁵ cells in a volume of 100 μL saline was implanted subcutaneously on the right flank of the animal near the axillary region. Animals were then randomly assigned to treatment groups. Treatment was initiated on day 4 after CT26 tumor cell inoculation to allow for a sufficient treatment window. MEK162 was administered once daily (QD) for 14 consecutive days by oral gavage (PO) at 30 mg/kg and anti-PD1 antibody was administered twice weekly by intraperitoneal injection (IP) (Table 4).

TABLE 4
Treatment Schedule

| Compound | Schedule | Route | Group Size |
|---|---|---|---|
| Vehicle | QD(1-14) | PO | 10 |
| 30 mg/kg MEK162 | QD(1-14) | PO | 10 |
| 100 μg RMP1-14 | D 1, 4, 8, 11 | IP | 10 |
| (Anti-PD1 | | | |
| Antibody) | | | |
| Combo | QD(1-14) | PO | 10 |
| (concomitant) | D 1, 4, 8, 11 | IP | |
| 30 mg/kg MEK162 | | | |
| 100 μg Anti-PD1 | | | |
| Combo | D 1, 4, 8, 11; | IP | 10 |
| (sequential) | QDx14 at 400 mm³ | PO | |
| 100 μg Anti-PD-1 | | | |
| 30 mg/kg MEK162 | | | |

The combination was evaluated as concomitant administration or sequential where MEK162 treatment was initiated at the time that tumor resistance from anti-PD1 treatment emerged. For the sequential treatment group, animals were enrolled in MEK162 treatment when tumors reached 350-400 mm³ after anti-PD1 antibody therapy was initiated.

Animals were monitored for tumor growth and body weight 2-3 times per week based on outgrowth kinetics. Tumor diameter was measured with digital calipers, and the tumor volume in mm³ was calculated by the formula: Volume=((width)²×length)/2. TGI was calculated on day 15. Animals were removed from study if found moribund or if tumor volume exceeded 1400 mm³. At that time, tumor tissue was harvested and frozen for later analysis of T-cell clonality by immune sequencing (Adaptive Biotech, San Diego, Calif.). Food, water, temperature and humidity were according to Pharmacology Testing Facility performance standards (SOP's) which are in accordance with the 1996 Guide for the Care and Use of Laboratory Animals (NRC) and AAALAC-International.

The murine CT26 cell line (a.k.a. Colon 26 or Colon Tumor #26) was developed in 1975 by exposing BALB/c mice to N-nitroso-N-methylurethane-(NNMU) (Corbett et al., 1975). The undifferentiated colon carcinoma cell line with fibroblast morphology was isolated from BALB/c mice. Extensive genomics, immune phenotyping and therapeutic background are available on this cell line (information summarized from Bhadury et al., 2013, Castle et al., 2014). The cell line is reported to harbor a KRas G12D mutation and the in-house cell line was sequenced and found to match the reported mutation (BGI, Cambridge, Mass.). Literature reports expression of MHC class I but not II and a mutational load of 1688 non-synonymous point mutations; 154 are both in expressed genes and in peptides predicted to bind MHC. The cells also reportedly have high expression of mutant gp70 (product of the envelope gene of murine leukemia virus (MuLV)-related cell surface antigen, known model antigen for studying antigen-specific immune response).

Cells were grown in a humidified atmosphere of 5% CO₂ and using RPMI-1640 growth media (Gibco Life Technologies, 11875-093) supplemented with 10% fetal bovine serum (HyClone SH30088.03, Logan, Utah), 100 U/mL penicillin, 100 μg/mL streptomycin (Gibco Life Technologies, 15140-122) and 2 mM Glutamax (Gibco Life Technologies, 35050). Cells were confirmed murine virus and mycoplasma negative (IDEXX Laboratories Inc, Westminster, Colo.) prior to implantation.

The mean values for tumor volume and body weight by study day for each experimental group were plotted including error bars for the standard error of the mean. % Tumor Growth Inhibition (% TGI): % TGI=100(1−Wt/Wc); Wt is the mean tumor volume of the treated group on day X; We is the mean tumor volume of controls on day X, where X is the last day that the control group is available in its entirety. Survival defined as morbidity, mortality or tumor size exceeding 1400 mm3. Animals that were moribund or found dead for reasons unrelated to tumor or study drug administration (e.g. gavage trauma, etc.) were censored. Cures: Animals with no palpable tumor at the end of the study were scored as cures. Maximum % Body Weight Loss (% BWL): % BWL=100(1−BWt/BW0); BW0 is group mean body weight at study start and BWt is group mean or median body weight on day where maximal body weight loss is observed. Tx-Related Deaths: Death occurring during live phase specifically due to drug administration and not attributable to other causes (e.g. gavage trauma, tumor-related morbidity, etc.)

Male BALB/c mice bearing KRas mutant CT26 syngeneic tumors were administered test articles, MEK162 and anti-PD1 (anti-murine PD-1, RMP1-14), as single agents or in combination. MEK162 was administered once daily (QD) for 14 consecutive days by oral gavage (PO) at 30 mg/kg and anti-PD1 was administered twice weekly by intraperitoneal injection (IP). The combination was evaluated as concomitant administration or sequential where MEK162 treatment was initiated at the time that tumor resistance from anti-PD1 treatment emerged. The doses and schedules employed in this study were well tolerated in all groups with <1% maximum body weight loss and no deaths attributed to test article administration.

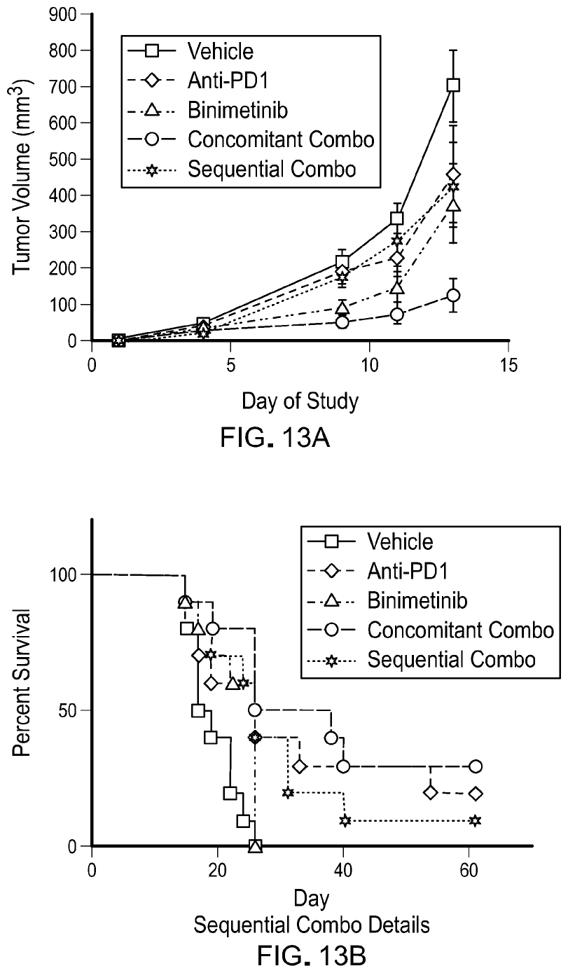

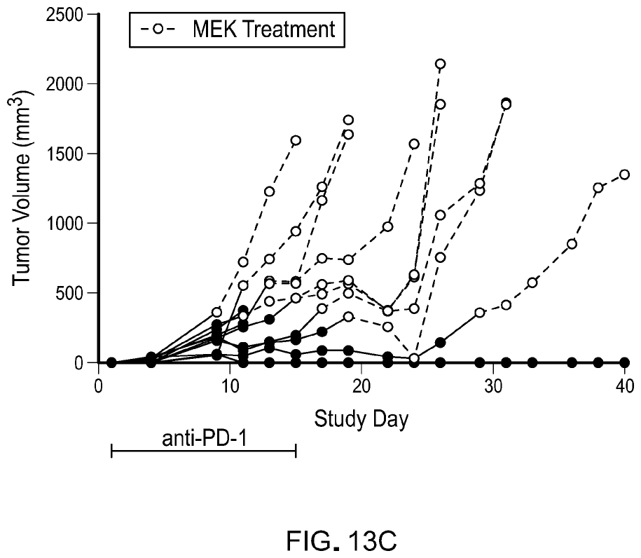

MEK162 and anti-PD1 were not highly effective as single agents in this model resulting in modest tumor growth inhibition (<50% tumor growth inhibition (TGI)) and median survival improvement (26 days versus 18 days for vehicle control) (Table 5). MEK162 treatment resulted in 43% tumor growth inhibition (TGI), 26 days median survival compared to 18 days for vehicle treated animals and no cures (FIGS. 13A-B). Anti-PD1 antibody treatment was similar with 41% TGI, 26 days median survival but 2/10 animals had no palpable tumor at study end (cure).

When administered in concomitant combination, there was marked improvement in activity (85% TGI, 32 days median survival). When administered sequentially (anti-PD1 then MEK162 when tumor regrew to 400 mm³), activity was similar to single agent groups (44% TGI, 26 days survival, 1/10 cures). The specific effect of MEK162 activity on individual animal tumor growth when administered following anti-PD1antibody resistance emerged was modest. Only 3 animals showed any decrease in tumor size when MEK162 therapy was initiated (FIG. 13C). The concomitant combination further had the highest number of animals with no palpable tumors of all groups at study end (FIGS. 13A-B).

TABLE 5
Summary of Results for CT26 tumors

| | % TGI | Median | Cures | Max % | |
|---|---|---|---|---|---|
| Group | (Day 13) | Survival | (day 61) | BWL | Deaths |
| Vehicle | N/A | 18 | 0/10 | N/A | 0/10 |
| Binimetinib | 43 | 26 | 0/10 | 0.6 | 0/10 |
| Anti-PD1 | 41 | 26 | 2/10 | N/A | 1/10 |
| Concomitant combo | 85 | 32 | 3/10 | 0.1 | 0/10 |
| Sequential combo | 44 | 26 | 1/10 | N/A | 0/10 |

### Example 3. Effect of Binimetinib on T Cell Repertoire

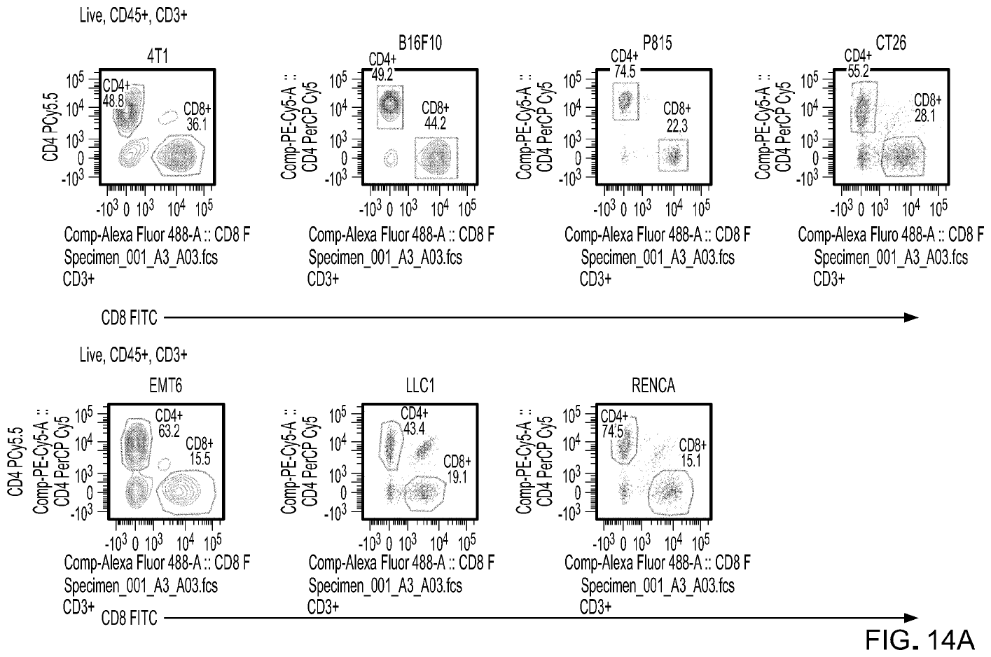

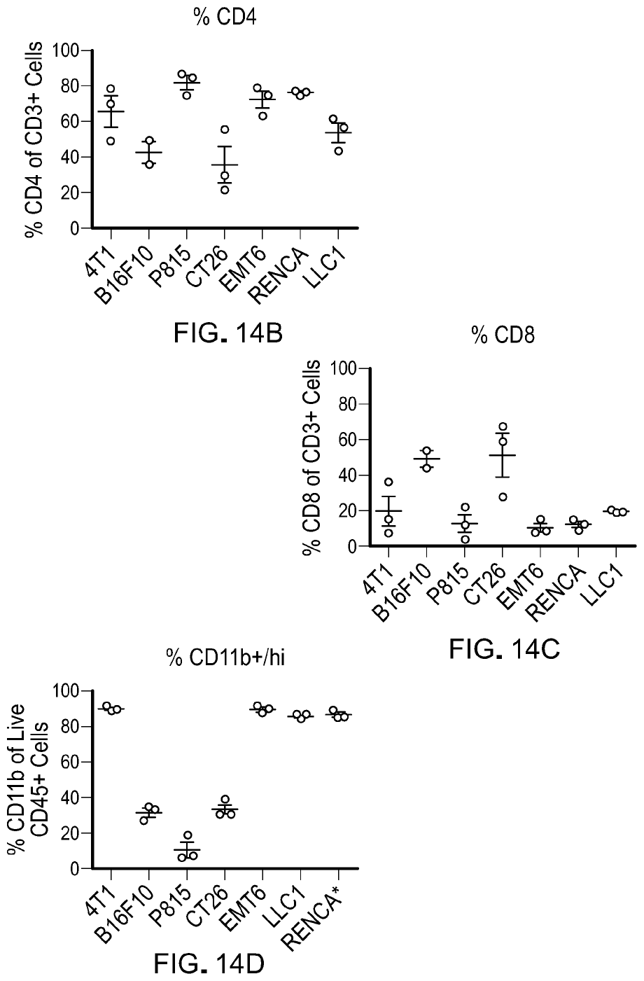

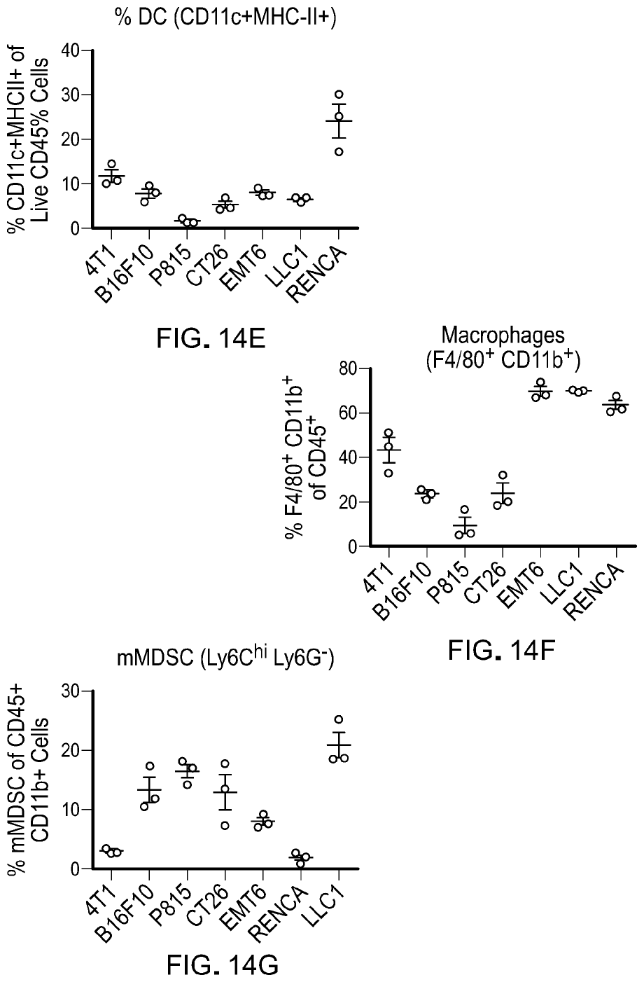

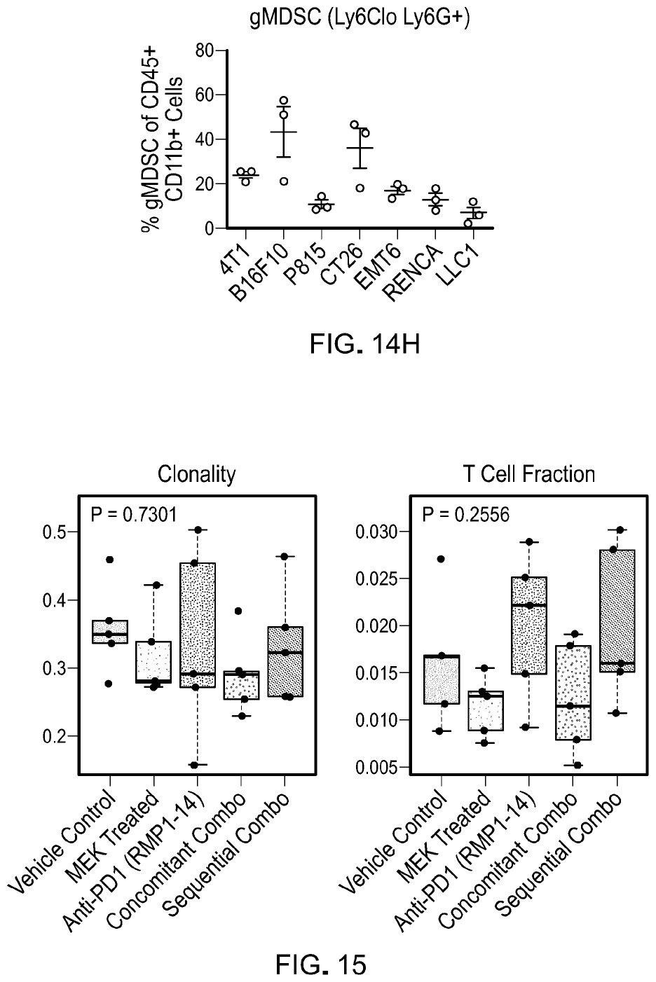

Flow cytometry analysis was conducted to phenotype tumor immune infiltrated across various syngeneic models: mice that developed 4T1 tumors, B16F10 tumors, P815 tumors, CT26 tumors, EMT6 tumors, LLC1 tumors and RENCA tumors. As shown in FIGS. 14A-H, phenotyping of immune infiltrates identified heterogeneity across the syngeneic mouse models.

The mechanism of combination activity was investigated by analyzing T-cell fraction and clonality by immune sequencing in 5/10 tumors from each group (FIG. 15). To determine the effect of binimetinib on the T cell repertoire of CT26 tumors, analysis was performed of mouse T-cell diversity, clonality and abundance as potential biomarkers by survey level sequencing of mouse tumor samples using the mmTCRB assay. Clonality was determined by quantitating the extent of mono- or oligoclonal expansion by measuring the shape of the clone frequency distribution. Values range from 0 to 1, where values approaching 1 indicate a nearly monoclonal population (clonality=1−Pielou's eveness).

As shown in FIG. 15, results suggested a trend for increased T-cell fraction in anti-PD1 and sequential combo groups. In contrast, MEK162 single agent and concomitant therapy with anti-PD1 antibody showed a trend for decreased T-cell clonality and T-cell fraction. These data did not support the hypothesis that MEK162 improved T-cell clonality in combination with anti-PD1 in this model as the driving mechanism of activity. Overall, these data suggested that concomitant administration of MEK162 with anti-PD1 was superior to sequential administration in KRas mutant CT26 tumors, and further, that the activity observed was not directly due to improvements in the T-cell fraction or clonality.

### Example 4. Inhibition of Programmed Cell Death Protein 1 (PD-1)/Programmed Death-Ligand (PD-L1) Pathway Increased Immune Mediated Anti-Tumor Activity

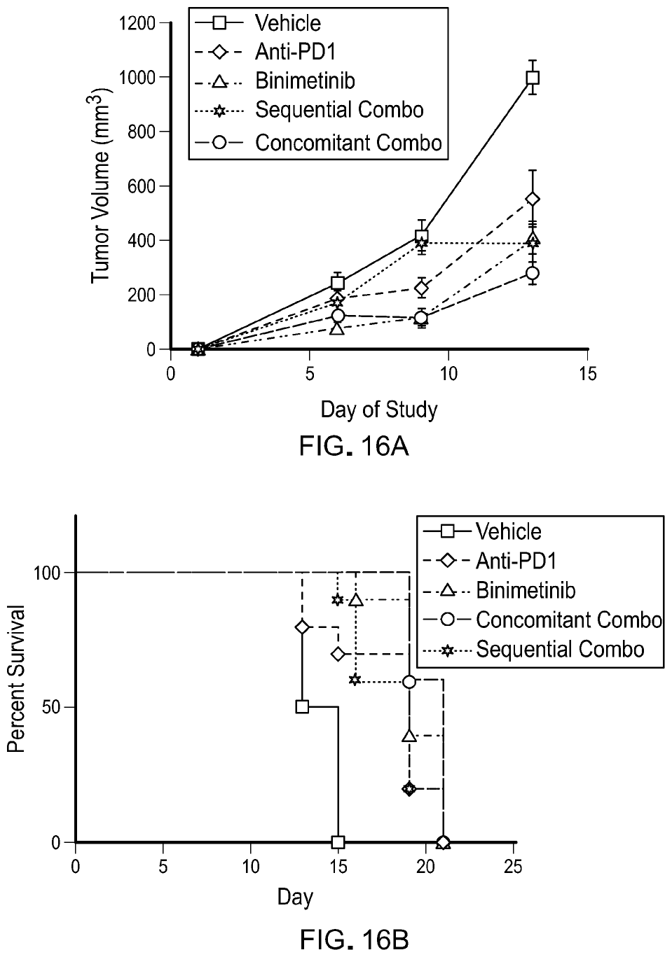

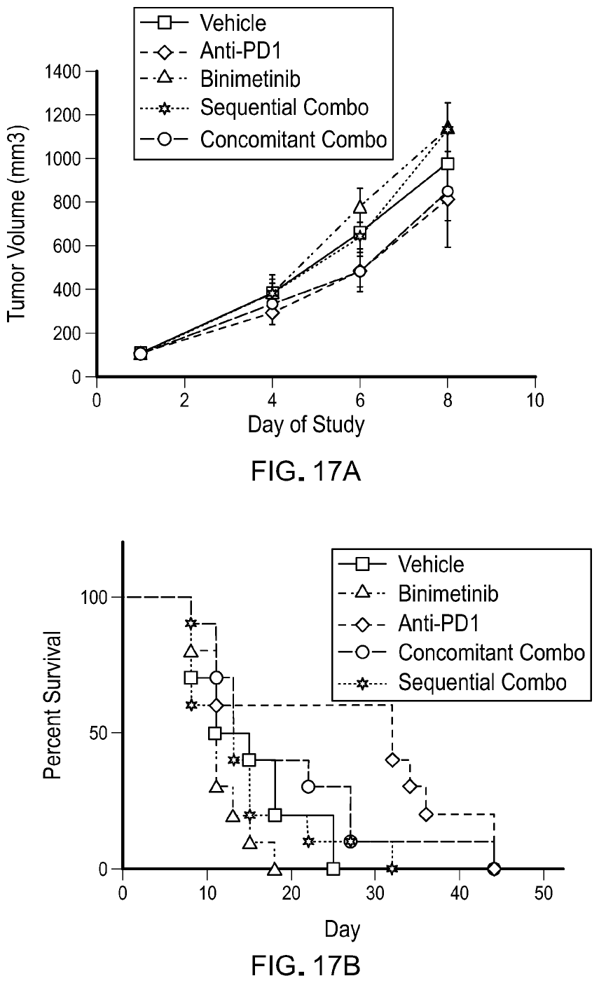

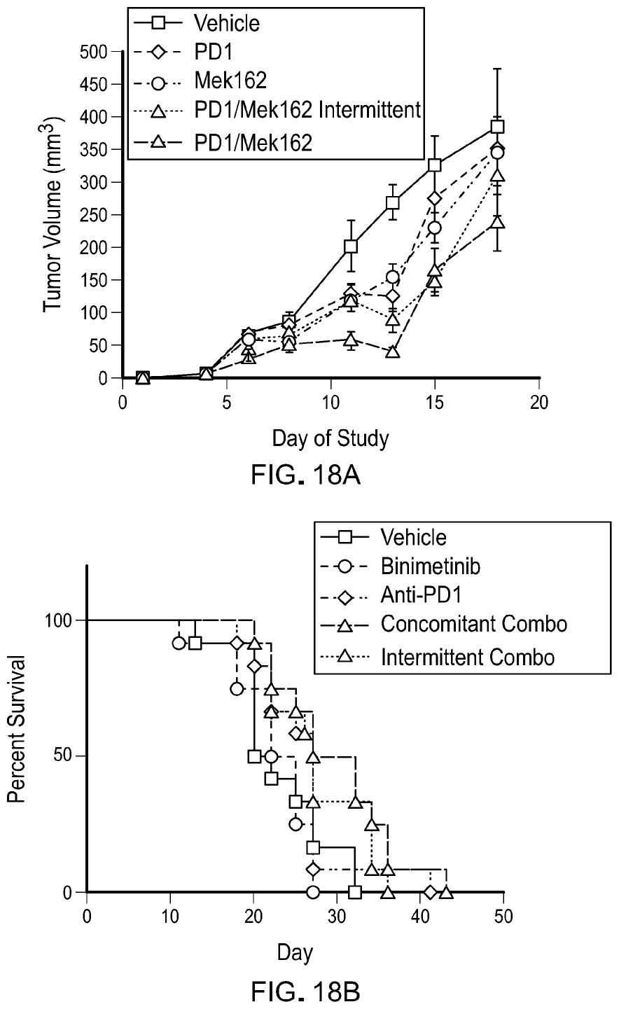

The study described in Example 2 was also conducted using B16F10 melanoma cells, Cloudman S91 melanoma cells and RENCA renal carcinoma cells (FIGS. 16-18 and Tables 6-8). For mice that were injected subcutaneously with RENCA cells, retired female breeders were used (23 weeks old). 200 μg of αPD-1 was administered on days 1, 4, 8 and 11. All other experimental procedures were identical to those used in Example 2.

TABLE 6
Summary of Results for B16F10 tumors

| | % TGI | Median | Cures | Max % | |
|---|---|---|---|---|---|
| Group | (day 13) | Survival | (day 61) | BWL | Deaths |
| Vehicle | — | 14 | 0/10 | — | 0/10 |
| Binimetinib | 59.0 | 19 | 0/10 | 0.8 | 2/10 |
| Anti-PD1 | 44.6 | 19 | 0/10 | — | 0/10 |
| Concomitant combo | 72.1 | 21 | 0/10 | 0.8 | 0/10 |
| Sequential combo | 60.9 | 19 | 0/10 | — | 0/10 |

TABLE 7
Summary of Results for Cloudman S91 tumors

| | % TGI | Median | Cures | Max % | |
|---|---|---|---|---|---|
| Group | (day 8) | Survival | (day 44) | BWL | Deaths |
| Vehicle | N/A | 13 | 0/10 | 12.4 | 0/10 |
| Binimetinib | −17 | 11 | 0/10 | 4.8 | 0/10 |
| Anti-PD1 | 16 | 32 | 0/10 | 12.4 | 0/10 |
| Concomitant combo | 13 | 13 | 0/10 | 20.7 | 0/10 |
| Sequential combo | −15 | 13 | 0/10 | 3.4 | 0/10 |

TABLE 8
Summary of Results for RENCA tumors
using retired mice (23 weeks of age)

| | % TGI | Median | Cures | Maximum % | |
|---|---|---|---|---|---|
| Group | (day 18) | Survival | (day 43) | BWL | Deaths |
| Vehicle | — | 21 | 0/12 | 15.1 | 9/12 |
| Anti-PD1 | 8 | 27 | 0/12 | 15.7 | 8/12 |
| MEK162 | 25 | 24 | 0/12 | 23.3 | 10/12 |
| PD1/MEK162 | 18 | 27 | 0/12 | 15.7 | 10/12 |
| intermittent | | | | | |
| PD1/MEK162 | 38 | 30 | 0/12 | 15.8 | 9/12 |
| continuous | | | | | |

These studies recapitulated the findings of Example 2, suggesting that MEK inhibition with MEK162 increased tumor responsiveness to immunotherapy. MEK inhibition with MEK162 may increase the number of active immune cells in the tumor, such as CD8+ cells, by inhibition of activation induced cell death (Ebert et al., Immunity 2016, 44: 609-621). MEK inhibition with MEK162 may also reduce the expression of immune suppressive factors in the tumor microenvironment, increase the expression of HLA-class I molecules and enhance tumor cell killing, which may lead to the release of tumor antigens.

This data supports the hypothesis that MEK inhibition with MEK162 could enhance anti-tumor immune responses. Thus, MEK inhibition with MEK162 led to an enhancement of anti-tumor activity when MEK inhibition follows, or is combined with, immuno-oncology (10) therapy.

1. A method for treating cancer consisting essentially of administering to a patient in need thereof, over a period of time, therapeutic agents that consist essentially of an amount of a PD-1 binding antagonist and an amount of a MEK inhibitor which is binimetinib or a pharmaceutically acceptable salt thereof, wherein the amounts together are effective in treating cancer.

2. The method of claim 1, wherein said cancer is colorectal cancer or pancreatic cancer.

3. The method of claim 2, wherein said cancer is colorectal cancer.

4. The method of claim 3, wherein said cancer is metastatic colorectal cancer.

5. The method of claim 4, wherein said colorectal cancer is metastatic microsatellite stable colorectal cancer.

6. The method of claim 2, wherein the cancer is pancreatic cancer.

7. The method of claim 1, wherein the PD-1 binding antagonist is an anti PD-1 antibody.

8. The method of claim 7, wherein the anti PD-1 antibody is nivolumab or a biosimilar thereof.

9. The method of claim 8, wherein nivolumab or the biosimilar thereof is administered intravenously every two weeks during the period of time.

10. The method of claim 9, wherein nivolumab or the biosimilar thereof is administered intravenously every two weeks at a dose of about 3 mg/kg or as a flat dose of about 240 mg during the period of time.

11. The method of claim 10, wherein the anti PD-1 antibody is pembrolizumab or a biosimilar thereof.

12. The method of claim 11, wherein pembrolizumab or the biosimilar thereof is administered intravenously every three weeks during the period of time.

13. The method of claim 12, wherein pembrolizumab or the biosimilar thereof is administered intravenously at a dose of about 2 mg/kg or as a flat dose of about 200 mg during the period of time.

14. The method according to claim 1, wherein the MEK inhibitor is crystallized binimetinib.

15. The method according to claim 1, wherein binimetinib or a pharmaceutically acceptable salt thereof is administered orally in the amount of about 30 mg BID or about 45 mg BID during the period of time.

16. The method according to claim 1, wherein binimetinib or a pharmaceutically acceptable salt thereof is administered orally in the amount of about 30 mg BID or about 45 mg BID for three weeks on and one week off in at least one treatment cycle of 28 days during the period of time.

17. The method according to claim 1, wherein, prior to the period of time, the patient was treated with therapeutic agents that did not consist essentially of a PD-1 binding antagonist and an amount of a MEK inhibitor which is binimetinib or a pharmaceutically acceptable salt thereof.

18. The method of claim 1, wherein the patient is not administered a BRAF kinase inhibitor during the period of time.

19. The method of claim 1, wherein, prior to the period of time, the patient was treated with a chemotherapy is selected from one or more of a platinum-based chemotherapy and a fluoropyrimidine-containing therapy.

20. The method according to claim 1, wherein the method further comprises assessing efficacy of treatment during the period of time by determining one or more of inhibition of disease progression, inhibition of tumor growth, reduction of primary tumor, relief of tumor-related symptoms, inhibition of tumor secreted factors, delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), increased Overall Survival (OS) or increased Duration of Response (DOR).

21. A combination therapy method consisting essentially of administering, over a period of time, to a patient in need thereof, therapeutic agents that consist essentially of therapeutically effective amounts, independently or in combination, of:
a MEK inhibitor, which is binimetinib or a pharmaceutically acceptable salt thereof; and
a PD-1 binding antagonist.

22. The combination therapy method according to claim 21, wherein the PD-1 binding antagonist is an anti PD-1 antibody.

23. The combination therapy method according to claim 22, wherein the anti PD-1 antibody is nivolumab or a biosimilar thereof.

24. The combination therapy method according to claim 22, wherein the anti PD-1 antibody is pembrolizumab or a biosimilar thereof.

25. The combination therapy method according to claim 21, wherein said MEK inhibitor is administered to the patient as a tablet during the period of time.

26. The combination therapy method according to claim 25, wherein said tablet comprises 15 mg of said MEK inhibitor.

27. The combination therapy according to claim 21, wherein said anti PD-1 antibody is administered by intravenous administration during the period of time.

28. The combination therapy method according to claim 21, wherein the patient has a cancer.

29. The combination therapy method according to claim 28, wherein said cancer is colorectal cancer or pancreatic cancer.

30. The combination therapy method according to claim 29, wherein said cancer is colorectal cancer.

31. The combination therapy method according to claim 30, wherein said cancer is metastatic colorectal cancer.

32. The combination therapy method according to claim 31, wherein said colorectal cancer is metastatic microsatellite stable colorectal cancer.

33. The combination therapy method according to claim 29, wherein said cancer is pancreatic cancer.
